SEQUENCE LISTING (63 sequences)

SEQ ID NO: 1 - DNA, 1306 bp, Mouse
acatttagagacttgctcttgcactaccaaagccacaaggcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagactctaattttgaataaatggatcttattcg

SEQ ID NO: 2 - protein, 178 aa, Mouse
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 3 - DNA, 1630 bp, human
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 4 - protein, 178 aa, human
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - DNA, 3903 bp, Artificial Sequence
  the sequence of the 5 arm
tggagttgtatggagttgtggttgggacctggaaacgaaactctagtccgcagaaggagcacaacactcttcaggggtgagccatctcttcagtctcccaaaataactactaacaaccccaccttctctatccataccacactcttgccgtgatgactctattgccagacaatctggctgatccctttgcctttccctggcagctccggtgtgccactcagagctcccagactgtcccctagaatggcagaaagtcatggagcaagtacatcaagaaagatgtcattttgttgggcgtgagtagattcctcataagatcttgtacactgtctataaaactctaaaatttactcatatgcacagaagcacacacacacacagagagagacacagacatatgtagacacacatacttttctacccagagtcttctgcagctaaaatggtgaagtttaccaagcattagggtgtcctattccaacgtgccactcaacttggacatcttgcaatgaagattggagaagaagagagagataatgtagggcttgataacgtgtgagtgcaataacagccatggactgctgacccctcaagctgggtgtgaagttcacctgtggtcctaggtactccagaggctaagacaggaagagagtaagttcaaggttatcctcagctacagggggaacttcttccgaatgaactaacaacaaatactgccttttttgtgtgtgtgtgtgatttgacagtatatatttattttttttccatttttattaggtatttagctcatttacatttccaatgctataccaaaagtcccccatacccacccacccccactcccctacccacccactccccctttttggccctggcgttcccctgtactggggcatataaagtttgcatgtccaatgggcctctctttccagtgatggccgactaggccatcttttgatacatatgcagctagagtcaaatactgctttttaaagccatgcttaataaaacttcctttcattggcagctgactagaaataggaaatattttgaaacgtgagtaatagaggagacattgccctccagatccccctccccctgcccacgacctccatctgatacatacctccctgatcctagggaaaatgtatttaaacacgacttctggactgactcaggccatttgtccccaaggaatgcacagactgatgtgtaacagtcatgtcctcttgaatccgcagctccgacagcacccaggctgttgcccatcttttccccagacatacgctctcacttccctgtcatgggcacttcccttgtcctgaaacccagctcaaatctcctgccatgagactcttgacatttatgtcaagatatagctgtggctttggggcttttcccagtgtgcgccaaacacttctcacagacttgagctctctgcatggtccttacttggctaaatatggacttgtatcaaggacctcacataaggttcttgagggccatttagagtctgaaatgttgaggagagagtgagcaacaacttgaagccaatgggggaaagaaaagggcaactagctgacaaatggctgaggataactggctatgcgtttcagaacaaataaaaaattcagatcataatcataaacccacttagttgaatatatattaacaagaaatataaatagttttcacatatctctcttctttctgtcattgtgggagtgtggagtatatacatttataacattaatacagtaatacatgacatagtatattcatgaaactaattatatttatattagtatatgctcagacactattaaaatattaatataaatatgatgtatcattatattgatataaattatgtgctataattaagtacatgctaatatattaatgttttatgttaacatattatgaaatagttttatgtacatgtgtaaaaggatattctagacaaaaaaaaataattacgggaagatataagaggtgctgcttctcctgctgagttctgtgctcacccctctgcttctcctggtgtttcatctcctacctctggatcaagctcatagaactgtccagtgccagtcccaaggacagctggtaccttctataggccagattcagttctgggggggggggggtcagacacacaaagctaaacacaatatgttcagggaccttgtagctgctcagaaataaaaaaaaaaacatgcaggaaggcagcaaaggggattgtggctatcaccgtgcagtaagtacaggtcacagtcaggagagagggcagtgagggtccatgctagctgggtcttgagcctcttctggggttcagatctctgatctacagcagtgtgtccacacctaaaacatcagctcagagaggcagttgcttctgctgttggaaacggacatcccaaaaaaaaacaaaaaacagaaatcaaaagggaaggagaaagtgaaagggatggaggcagcttgtccccttccctgtgcttgctgctggtagaaaactcagcctggaactgaccggagcagcagttcttgagtcaattccattccaacttctagaagattcttttcccgtcgaagagtgtcaggaggagaggccagacccccttgatcctgatctgccagccactgcatcagataagacgagataaccccgagttcctgttctaccagccctggtgtggtaaccctctccaatggggcaggcttggaaccctgtgccaacgaagatcctcccccgtactgatgcaggaaggacagcccgggagtgtaccctctacatgggtctacttttatttaagcaaacattccctggtcaacaggacgtgtagcattgccccccccccttgggtcacacagaaaacaggtaccaggaggacaagtagttgcttgcccagggtacagaatgaaaggcaataggggactctaggcgaatgttcttcccacccaaactgaggtagtaggagaagtccctactgaagggaaggtccagacataatcaaaggactaccagagatctcccaggtatctgtagaagtactaacatctccatccttcaacagctacaggttacacgtctccaaggctgggacattgtaaaacagggccatggtaaggtctacccgacagcacagagcaagcctcccagaagtctgagttccttctcctaacttctcatgctgggatctgagcttcttcgtgaaacacggggcagaggaggcaccagaactctcctctgaccaactgccccacagcacacatatcctcaaaggatagtcttgaatacgtgatggaagaattaaagagagtgaggtctgaagaaaatcagccctctcggggtttcctttgggtaactgagtgctaaggtgacttccgagtcagcaagaaatatcggacgttcaacccaggttgagtggaggaaacaattatttctcaatcctaatatgttctggaatagcccatttatccacgtcattatgacctgggagtgcgtgaatggaatccacagatgagggcctctgtacatagaacagctgtctgcctcaggaaatacaacttttagtattgagaagctaaaaagaaaaaaaaattaaaagagaggtagcccatactaaaaatagctgtaatgcagaagttcattccgaccagttctttagcgcttacaatgcaaaaaaaagggaaaggaaaaaaaaaaagaaagaaattaaactcaaaaattgcatggtttagaagagggaggaggagcctgaataacaaaaacctttgccaggaaggccccactgagccttcagtataaaagggggaccaagaacaggaggtctacatttagagacttgctcttgcactaccaaagccacaaggcagccttgcagaaaagagagctccatc

SEQ ID NO: 6 - DNA, 4527 bp, Artificial Sequence
  the sequence of the 3 arm
gttaaaaatggaagctaggggcaggagggtggggagatggctcagcagttagagtgtgtgccggcaccagtgagacaggagttcatatccttggaatccaaccaacacacaaccaacactaggcaagagatgcctaatgtgtcagtggttgcttcaagctccagaggtcctggggtgaacttagatctgtcttgggggcgctctctggggattaacacaaattcatcactagtttctagaactgggaaatcgtctggatactactggcaatcacaattaaaaactcctgccaagcctgccaaaccctttcacatgctaagcaaacatctgctgctcaactaggtctccagcctcttgacactttgagataaggcctcaaggcagttcagtatggccttgaactcagctcttcctgcctccgctgcccaagtactgggactcttcttcaaactgaggtcacaggcagcagagttgagccattagggttggatagaggtaaacaaacctactcagctcccaaaggatgcaaccactcttcctgccccctccctgagccaccagatagatattagactgacttctgcctggggtagggggagtggctggggaagccctactaaatgtctcgtgactttgaggataaggggaaataatgagctgagtaaccctaacaccacatacacacacacacacacacacacacacacacacacacacacgtgctggccaggcagcccgggacagggtgggtgggagctgacaagctgtggtcattttttcagtaagacctgactcacttcagttaaagtaaccatcggaaggaagtgggaagcaccaaagctgaaactgtgagatgaaaggttagggttaagaatggaagctggggggggggggggggggcgggggcagagagtgggatggctcagtagtttgagtgcttgcgggcaccagtgagacaggagttcatatccttgaaatccaagcaaatgctagtgtgcatggtggcctgagaaggcacagacaggggattccaagagcaaagtggctatctattaagttccaaaccctggctaagcagctgaggtgtctatttaacatatcaaagcagaaccggcctccaggttctcccagcatccctcagtccctacctgttaaggtttatggctggcataccctgccctctatcctgaactctctagcccagaggttcttccttatataattcagacattttggttaactgcccttggccattttggagagctctggattcgagtctacctcagtgagaagatggaagaggaattgagggtgattccgcacgtcaatgtcaggcttgcatgtggacatgtgtaaccacccatgtatgcacccatacacatacaaagagtgcatgcacacatccatgatgaagaaatgaagaagaacaagaagaaaaagaacaagaagaggaggaagaaggaggaggaggcgccggctggtgactaagctctcagcctgatccagagaagctctaagccctgatctagaaaaatcccaacagctcgggctcagtggcagccagcacatgataactagtcagaaatggtctctcactcaagagactgggcattggaaatgatagaagcaggtctagaaaggccgcataactgatctcaacagtagctacaatttgactggaacatcttctgggtgtgtcagcaccacacttattgctctatgaagcacactatattgcttttaggcttgctatttcctgtagattcagaagtgtgagctttttcactattcagtttgctgcactatacagttcagtaattcaacattctgcaaattgtgtcagaaattcaatttttaaattactttattaattctcaaccatgtgttgatcataaattaatcttcaaaaatacctaggctctctgtttgctggttaaactctgtgtgtatctatctctggacttttatacctaatatttacattaataataatgtgaacactaattaaagccatttgctgactactttactatgcactacatatatactaacttatgatcatgtaacattatgaggcactattaaattgatatttcaaaattttttgagacagagttttactgtatatccctaactagcttgtaactcactatgaagactaggtggccttgaactcacaaagagccacctgcctctgcctcccaaatgctgggattaaaggtgtgtgccatcacatctagccaacatctcaatttcttacacatgaagatgagattcagttaaaacatctaccttaggtcaaccaactactaatggcagagttcatgatcttaatgattatatcaaacccttatatatcattttcaaacctaaattactgaaaatttctctttttcctgtattgttctgcctccttaagagttacagatatttttccagctggtagtgtgcacctataatgctaggattgaaaagctgaaacacagaaattaagaatatgaagactgcctgggctacactgccagaccctgcctcagaaacaatcataaaatactatacactcactcccatatgctcaagaggctgaagaaggaaggtcactgaatccaggagttcgaggccagcctctgaaccctgtaacaaaaataataatatacactttagtattatgtgtctccattcctatttgtctcaaaatatttttatttctcttctaattactctttgactatctggttcttcaagagcaggagaactgttcacatgcctaatatagctcatcacacagctctttgaagatgtctgcatgagtgtccacagcacaggatatatacagcttgccatgagtcactgatctgggagagaagtaagaggaaggtaggtacagcatgatttctggcctattccaggaactctcacaaccactggtcctaccacattctacctgttagaaggggtaaagtacagaccacgtacaagtgaggagcagttgggcttcaccttttgaaggaagaaatcaaaagaatctgtgtacctgtttgtcttagttaaggctttactgctgtgaacagacaccatgaccaaggcaactcttataaaggacagcatttaattgaggctggcttacaggttcagaggttcagtccattatcatcaaggcaggaacatggcagcatccaggcagccatggtacaagaggagtagagagttctacatcttcatctgaaggctgctagcagagtactgacttccaggcatcgaggatgagggtcttaaagcccacacccacaatgacacacctactccaacaaggccacacctcctaatagtaccactccctgggtcaagcatatacaaaccatgacactattttaacatttctctgccctttaagctctgtgttcttggcaagtggatggaaggaagagctggctgagaacagtccgaaaggctgggccatcaacacagacattgtcatttacgtccccctcttgtagacttagcctccatttctcctttggaaatttgaaaaatcttggcctgcatgctaggtattgaatgtttgtgtcccacttctcccaaccccaaccaaattcatactgactcccaaagcaatgacatttgaagatagggtctttgggagatgaccaaggttagatgggacaacaagaatagaaccttcatggtggacttagcactaggagagaccctgaagagtttgttctatttccctatcatcttttaggaatactcaaagaaaagattttacaagcactcaatgagataggacctccattgaagcaagagcagaggtctcaggatgcaaactgccttgctggcaccttgaatgtctcagcctccagagctgtaagaagataagttcctgcagggcaagctaagctgcctgcagtgtcttctgactacaggtcaagctctcacggacacatgaagtctggcatgaaggcatcacgtgtgtgaaaatatggaggtgttttgaagaggtgtgttacaattgaggtttgagtgagggtttggaaagagagttaagactttccgctctaagaccagctatctcatctccccagttgtaccaggtctagggtgacaggagatagtgagaatttgcattcatctgtggtcctggctactaagatgtggagggtgagcagcagtgagagaacttcctggaatctagataacaaagtgattgctttacatagatcttggcagaaattaggtaaaaatgaaaagcaaacagccccccctcccattctaggaggaaggtgccagtgccagatgagaaggaggacataggcaggcactggctcgatggcagaagccgtccattctgcaccatgtttctccttcaaacatacttttgcatttaattgtggtaatatttagggaacgtataaatgaaaactgagtcatgaaaacataaaactagattttaaaatgagaggaagatccttgccttccctctgtgagccacagaacctcctcaggaatgaaccagttgactccact

SEQ ID NO: 7 - DNA, 1306 bp, Artificial Sequence
  The mRNA sequence of the engineered mouse IL-10 after humanization
acatttagagacttgctcttgcactaccaaagccacaaggcagccttgcagaaaagagagctccatcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagactctaattttgaataaatggatcttattcg

SEQ ID NO: 8 - DNA, 75 bp, Artificial Sequence
  The connection between the 5′ end of the Neo cassette and the mouse IL-10 gene locus
aactctgagacgaaatgttgggctcgaggtcgacggtatcgataagcttgatatcgaattccgaagttcctattc

SEQ ID NO: 9 - DNA, 77 bp, Artificial Sequence
  The connection between the 3 end of the Neo cassette with the mouse IL-10 gene locus
tattctctagaaagtataggaacttcatcagtcaggtacataatggtggatccgttaaaaatggaagctaggggcag

SEQ ID NO: 10 - DNA, 24 bp, Artificial Sequence
  primer
tgcccaggtcactaaagcaggtta

SEQ ID NO: 11 - DNA, 25 bp, Artificial Sequence
  primer
gagcgccagcaggatcttataagtt

SEQ ID NO: 12 - DNA, 21 bp, Artificial Sequence
  primer
cgcattgtctgagtaggtgtc

SEQ ID NO: 13 - DNA, 25 bp, Artificial Sequence
  primer
gtctccagagtcaccacatgtgttg

SEQ ID NO: 14 - DNA, 27 bp, Artificial Sequence
  Probe
caaaacggttatctctgagtagcccga

SEQ ID NO: 15 - DNA, 24 bp, Artificial Sequence
  Probe
ggtggcttctctgtatccaacccc

SEQ ID NO: 16 - DNA, 24 bp, Artificial Sequence
  Probe
gggcaggagttccagcctgaatag

SEQ ID NO: 17 - DNA, 27 bp, Artificial Sequence
  Probe
agcttctggaaatttaagcccacccat

SEQ ID NO: 18 - DNA, 23 bp, Artificial Sequence
  Probe
ggatcggccattgaacaagatgg

SEQ ID NO: 19 - DNA, 23 bp, Artificial Sequence
  Probe
cagaagaactcgtcaagaaggcg

SEQ ID NO: 20 - DNA, 24 bp, Artificial Sequence
  primer
ggtttagaagagggaggaggagcc

SEQ ID NO: 21 - DNA, 28 bp, Artificial Sequence
  primer
tgcagttccatcagaatgcatatttcag

SEQ ID NO: 22 - DNA, 24 bp, Artificial Sequence
  primer
agatctcgaagcatgttaggcagg

SEQ ID NO: 23 - DNA, 24 bp, Artificial Sequence
  primer
gctgaaactctgagacgaaatgtt

SEQ ID NO: 24 - DNA, 25 bp, Artificial Sequence
  primer
cttgaagcaaccactgacacattag

SEQ ID NO: 25 - DNA, 3509 bp, Mouse
agtcttcagttctcaggacgccgccggccggaggcgtaaaggccggctccagtggacgatgccgctgtgcgcccaggatgttgtcgcgtttgctcccattcctcgtcacgatctccagcctgagcctagaattcattgcatacgggacagaactgccaagcccttcctatgtgtggtttgaagccagatttttccagcacatcctccactggaaacctatcccaaaccagtctgagagcacctactatgaagtggccctcaaacagtacggaaactcaacctggaatgacatccatatctgtagaaaggctcaggcattgtcctgtgatctcacaacgttcaccctggatctgtatcaccgaagctatggctaccgggccagagtccgggcagtggacaacagtcagtactccaactggaccaccactgagactcgcttcacagtggatgaagtgattctgacagtggatagcgtgactctgaaagcaatggacggcatcatctatgggacaatccatccccccaggcccacgataacccctgcaggggatgagtacgaacaagtcttcaaggatctccgagtttacaagatttccatccggaagttctcagaactaaagaatgcaaccaagagagtgaaacaggaaaccttcaccctcacggtccccataggggtgagaaagttttgtgtcaaggtgctgccccgcttggaatcccgaattaacaaggcagagtggtcggaggagcagtgtttacttatcacgacggagcagtatttcactgtgaccaacctgagcatcttagtcatatctatgctgctattctgtggaatcctggtctgtctggttctccagtggtacatccggcacccggggaagttgcctacagtcctggtcttcaagaagcctcacgacttcttcccagccaaccctctctgcccagaaactcccgatgccattcacatcgtggacctggaggttttcccaaaggtgtcactagagctgagagactcagtcctgcatggcagcaccgacagtggctttggcagtggtaaaccatcacttcagactgaagagtcccaattcctcctccctggctcccacccccagatacaggggactctgggaaaagaagagtctccagggctacaggccacctgtggggacaacacggacagtgggatctgcctgcaggagcccggcttacactccagcatggggcccgcctggaagcagcagcttggatatacccatcaggaccaggatgacagtgacgttaacctagtccagaactctccagggcagcctaagtacacacaggatgcatctgccttgggccatgtctgtctcctagaacctaaagcccctgaggagaaagaccaagtcatggtgacattccagggctaccagaaacagaccagatggaaggcagaggcagcaggcccagcagaatgcttggacgaagagattcccttgacagatgcctttgatcctgaacttggggtacacctgcaggatgatttggcttggcctccaccagctctggccgcaggttatttgaaacaggagtctcaagggatggcttctgctccaccagggacaccaagtagacagtggaatcaactgaccgaagagtggtcactcctgggtgtggttagctgtgaagatctaagcatagaaagttggaggtttgcccataaacttgaccctctggactgtggggcagcccctggtggcctcctggatagccttggctctaacctggtcaccctgccgttgatctccagcctgcaggtagaagaatgacagcggctaagagttatttgtattccagccatgcctgctcccctccctgtacctgggaggctcaggagtcaaagaaatatgtgggtccttttctgcagacctactgtgaccagctagccaggctccacggggcaaggaaaggccatcttgatacacgagtgtcaggtacatgagaggttgtggctagtctgctgagtgagggtctgtagataccagcagagctgagcaggattgacagagacctcctcatgcctcagggctggctcctacactggaaggacctgtgtttgggtgtaacctcagggctttctggatgtggtaagactgtaggtctgaagtcagctgagcctggatgtctgcggaggtgttggagtggctagcctgctacaggataaagggaaggctcaagagatagaagggcagagcatgagccaggtttaattttgtcctgtagagatggtccccagccaggatgggttacttgtggctgggagatcttggggtatacaccaccctgaatgatcagccagtcaattcagagctgtgtggcaaaagggactgagacccagaatttctgttcctcttgtgaggtgtctctgctacccatctgcagacagacatcttcatctttttactatggctgtgtcccctgaattaccagcagtggccaagccattactccctgctgctcactgttgtgacgtcagaccagaccagacgctgtctgtctgtgttagtacactaccctttaggtggcctttgggcttgagcactggcccaggcttaggacttatgtctgcttttgctgctaatctctaactgcagacccagagaacagggtgctgggctgacacctccgtgttcagctgtgtgacctccgaccagcagcttcctcaggggactaaaataatgactaggtcattcagaagtccctcatgctgaatgttaaccaaggtgcccctggggtgatagtttaggtcctgccaacctctgggttggaaggaagtggactacggaagccatctgtccccctggggagcttccacctcatgccagtgtttcagagatcttgtgggagcctagggccttgtgccaagggagctgctagtccctggggtctagggctggtccctgcctccctatactgcgtttgagacctgtcttcaaatggaggcagtttgcagcccctaagcaaggatgctgagagaagcagcaaggctgctgatccctgagcccagagtttctctgaagctttccaaatacagactgtgtgacggggtgaggccagccatgaactttggcatcctgccgagaaggtcatgaccctaatctggtacgagagctccttctggaactgggcaagctctttgagacccccctgggaacctttatttatttatttgctcacttatttattgaggaagcagcgtggcacaggcgcaaggctctgggtctctcaggaggtctagatttgcctgccctgtttctagctgtgtgaccttgggcaagtcacgtttcctcgtggagcctcagttttcctgtctgtatgcaaagcttggaaattgaaatgtacctgacgtgctccatccctaggagtgctgagtcccactgagaaagcgggcacagacgcctcaaatggaaccacaagtggtgtgtgttttcatcctaataaaaagtcaggtgttttgtggaaaaaaaaaaaaa

SEQ ID NO: 26 - protein, 575 aa, Mouse
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSILVISMLLFCGILVCLVLQWYIRHPGKLPTVLVFKKPHDFFPANPLCPETPDAIHIVDLEVFPKVSLELRDSVLHGSTDSGFGSGKPSLQTEESQFLLPGSHPQIQGTLGKEESPGLQATCGDNTDSGICLQEPGLHSSMGPAWKQQLGYTHQDQDDSDVNLVQNSPGQPKYTQDASALGHVCLLEPKAPEEKDQVMVTFQGYQKQTRWKAEAAGPAECLDEEIPLTDAFDPELGVHLQDDLAWPPPALAAGYLKQESQGMASAPPGTPSRQWNQLTEEWSLLGVVSCEDLSIESWRFAHKLDPLDCGAAPGGLLDSLGSNLVTLPLISSLQVEE

SEQ ID NO: 27 - DNA, 3672 bp, human
gtcagtcccagcccaagggtagctggaggcgcgcaggccggctccgctccggccccggacgatgcggcgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacagggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggcccctggggcagaagttaggcacgaggcagtctgggcacttttctgcaagtccactggggctggccccagccaggccctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatcttaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagccactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcgtagggatgtgaggttctgctgaggaaatgggtatgaatgtgccttgaacacaaagctctgtcaataagtgatacatgttttttattccaataaattgtcaagaccacaggaaaaaaaaaaaaaaaaaa

SEQ ID NO: 28 - protein, 578 aa, human
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 29 - DNA, 5705 bp, Artificial Sequence
  The upstream homologous arm (5′ homologous arm)
gaagaatgaggaaatggccccctcagaaacaggagaggtcagggccatttggggggtagaaggaagacacaacacttctttgaaatgactggggatatttttgtgtgtggtctgtatgccatctcttacatttgagttcacaaggcaaggagaacagataggcccataccctttcctgcttagatggtgtttccaaaaaccactcagatgaagatgggtagaagagaggtgtgctgtcatgcatagcacgtaccacagctggagagaagccaaacaaatccaggctgagagaccaggctctattagtacagcaggtggcatgttgtagattatttctactaaccacatctactaaccacatcttccccactggggaaggctctatctgtaggctccacattttttctgtgtgtgtttcaattaagcaatggctagtggaggcccagggcctgggctaagggcacacggctggcagtggataagctagaatgcggcttcctgttttcaaggcccatgagttccattttgagattcgatcaagagcagggtcctaggtgcatcctgcaaaactctacaggactggagaggctctgagcctgcgtctgctgcttcaaggcgtcagcaggggcaatctttctccttcacttgggagatcagcaattctccctttccctccaagccgccaggcaggacaagctacatagctacctggaggcaggggagaacttagctgtggaaagcatggtttgaattctgaagagtgagtgagtcaccaattacttacctgcaggctccgggttctgagtctatttaaatgcaaatctgttccccagctatctcctatctataaattcgaggttgtgtgtaagaaccaagttgattgtttatgggatggatccccaggtagggcagtttctggatggtcattccttcagtctctgctccgaactttgtctctgtagctcctcctgtgggtattttgtttccctttctaaggaggatcgaagtgtccatactttggtcttccttcttcttgactttcatgtgttttgcaaattgtatcttgggtattccaagcttctgggctaatatacacttatcagtgagtgcatttcatgtgtgtccttttgtgattgggttacctcacttgggatgatatcctccagatccatccaattgtcttagaatttcatgaattcattgtttttaatagctgagtagtactccattgtgtaaatgtaccacatttttctgtatccattcctctgttgagagacatctggattctttccagcttcttgctataaataaggctgttatgaacatagtggagcatgtgtccttattacatgttggagcatcttctgggtatatacccaggagtggtattgctggatcttctgttagaactatgtctagttttctgaggaacagccaaactgatttccagagtggttgtaccagcttgcaatcccactagttatggaggagtgttcctctttctccacatccttgccagtgatccatcccataaacaaccaccaaacccagactctgttgcagatgccagcaagagcttgctgacaggagcctgatatagctatctcctgagaggctctgccagtgcctgactaatacagaagtggatgctcatagtcatccattagacagagcacagggtccccaatgaaggagctagagaaagtacccaaaggagcttcagggtttgcagccccataggaggaacaacaatatgaactaaccagtaagcccagagctccctaggagtaaaccaccaatcaaagaaaacacacggagggattcatgactctagctgcatatgtatcataggatggcctagtcggtcatcaacgggaggagaggcccttcgttctgtgaaggttctatgccccagtataggggaatgtctgggccaggaagcgggagtgggtgggttgggggacagggtgagaggagagaggataggggattttcggaggggaaactaggaaaggggataacatttgaaatgtaaataaagaaaatatctaataagactaaaaagttagagttaaaaacaaaaacaaaaccagaacccagctgaagtgcatacagcaccttactccggcctgagctgctgctcagagggcgcctcttcggagccctgccatactgagcattagtattgggcagagagtgaaagggagcgggcatgaattagattctacagacacatttaaaagtttctcctgggacaggggctggactgagcatgcccagagcactggttacgccttggcagtccccataccttgcacttgaaatccttttcactgcaaggactgcccagtttcaccaggcagcttcctcctttctgtctccatctcagagccggtgcctctgggtttcactgttttgtgctgccctgacagagtgattgtaatcactgtagggcagcccgggacttcccaaacactcgtctggagaccagctgtgtgcctgcagaactcagtttcctgcagtgctcagatgccctgggaagtttatgtaagagtgtcatgaaagggagacagtgactccatcagacttcaattgtgccctaagaaagagagagctgcctcaagatcaagctatctacctggccgggtccatcggcttcctgggctcaccacggtaccatcatcctcccagccccaagttgccctcctcctcctcctcctgaacattctcattctctctcaactcacttggtattgtgctttgcaaaaaatcttccaaagttcgaagtcaccattgccaaaataggaaatacagaagttcaaggaccaactgagccctgcccgccaactaaagtctctgccctctgccaccgtgtccccatttgcaaaacactgacagctgcaccacctccccaccccccagtggattctgttaaggactggatgaggctgtgtttgcagatgaatcaggactggccatcatggctgtcaggaaagccctgaatctctgtggtgtaatacagggatccagtttaatgtttcatccttccttcagagttctccaagaagtgattcggggcacccaggtcttttgcattccctggaagtcctcggctggggccctctggatggctgacaggatagcacaagaggcagggcctgggtgtaccactctctgctgcagcttcacacgggtttaaacccggtcatattttccagccacacaccggaaaacagttgtcccatgttcccaggaaaaggacagggatttgtttgttcagtacctggctggtgtctgctatgcagtgtaaagtgttttatggagtccttgacacaccttaagagtccgagcatatgaaaatcctgttatttaccatctgttccaatatggatgctcacaaaagaacccctgaagaacaagctactcaacacaaaacagcatgcatgtgggcttcttctaattttttttctttcctcgggtatgataattatgtgcctaatgcagcttctccctgggtgctatgcaggaagcagggagtgtagcaggaagcccacactcaatgctggttctgccccgagcaagggccaaattatgtcagccctaggatgtgtgcccactttacagacttctctaaaactgctgtatcccctcatctccaatgtcccaatcccctccctatccatccatcaacttgcctattacaacgcagcgctcaagtctcagataaccggccacctctgccatggaaccttccttgttctcagccctgtgtccccttgttagtctgtactttccttcccagaaactgttaaaatctccgctcactttctgtctctggcaaggtggtttctagaggaaggggcctggttttggccagcatcagcttgtgactgtaaattctggtatgtggaagaaccatctcgatcgtcccttaaaacagccaaggtgcaaggctttaaaattcttataactcaaccaaaaactaaaaggaagacaactgaggaagaaacaccgcagtgtcctcttttttaagaggtaaacggggactaagaagtcatgcccgggtacttgcgtcacgcctgggtacttgtgtcacgccaagcagtatatgtgctttaccagcggaaggatgggcagtgcagaacagcaattaaaacaaagtcctcccacattaagaagtctgaagttttccaacatggctgacatgagggtattcacagtgacgagggtatccctgagtgtggttctcactgattgtgtgtgtgtgcgtgtgtgtgtgtgtgtgtgtgtgctcgatgctatggttcacagatattacttccaactagctgaatagctagggcagcccagtcctcacagacaagcacctttcccagtgagttccctcggcacagaacatgatcccactccacttggcatcctctgtctaggccactggacatagcactatccactcccacggtgtttagacaaagcttgtgtctccacaagccaagggggtggtgtgcatcactagctctcaggtcacgacggcagtgctcgtgtctgacacccccaagttggagggtccttctccaggttcagaggttgcccaaatctttgacatagtaacagtactttgtcatctacgaacttcacaccggagaaccatccaatccaatttccaggaagaaaaagaaaaatttaacaaatacctcatactgttttcagtcagtttctaatttgatgggaggcaggattggtattggcactgagaatcatgcaatttgcagaccgatggctggccaagccttctcaaggcagtagtgtcttccgtgtgacaggtaggctctgaggggaatagaaagtgacttcttgagagcaagggcatcctagtgactccagcccttccttggcttctgtgcctcctcagaacaaaaacagggcttccaggatagcatgaatatgtacctggcagatgaccccacgggcgctgtacacctcacaattagatccctgaaaatcatgtaagtgacatactcacagtatcagcgccacctgccaataagtcaggtggacaaagcttggtctctcacacaattaacatctttggtgacactggagggcagacagatgtcctggtcactgaagaggcatctgagtgataccaggtccctcaaggcgcctattgacacttctaacctgcagatttagggcaagaacaacctaaagggcctgggggtgtcatggactcccaccaagagtcaggtagggacgttccctcactgttggcttagcagagggccctgcatagaggtcccaggactttcacacatgtacctgcacgcttgtgctttgctcctggcccagtcagtaattccaattgctacaacagagaggggaagggggcaaggggaaggggagggggagggagaggggagacaggctgtccagtcggtgtgagagttaagagcccagccacagaaagcctcagctgctctttcaccttgagacaaatgccccagaactagctagacttcgctgcctgctgaggttcctgcggaggttcccaagctccgtgggctgagggcggagcaacgaggctccgccttcaggttgatgtcaagctggccctgtggatccagcggcagtcttcagttctcaggacgccgccggccggaggcgtaaaggccggctccagtggacgatgccgctgtgcgcccagg

SEQ ID NO: 30 - DNA, 6647 bp, Artificial Sequence
  The downstream homologous arm (3′ homologous arm)
ctgtgaaccggccactgtgtatcatgaattggtcctggtcattatcctcatttcacaagtgaggacagaggggttgcttccactcacaggatcacacagctaggaggagtactaggggaggctttgcagcatcccatcctggtgctgctgagggaatagactcagaaagacagaagagatggagtggtcacagagggaagggaacagtggagagctgacctagagaggagaggacaggatctctgtcccctctcagatggaggcatgtgtggcaagtcagcctcttgaggccatcctacaagaacaagtagaagtccccaaacctctggttgtctctgacctagtctcagactgtgtcagaagtggacagccacagactctggaccctgagtaatgtcttctaatattaatacataatagatggttacaggatatatatatatgcaagtatatatatatcctgtaaccatctattataacacacacatatatatgtgtgtgtgtgtgtgtatgtgtatatgtgtgtgtgtatatatataaatatatatatgtacactcatacacacacacacacacacacacacaccctggcctaatcagggcctttgcctagacaagagtctgttctcacccagcccttaaagctctcaagggtttaatagttcttccttgcatctcgctgaagtaggtcctcatcgtctttctttgcgctcctgggagattcagtgccccgtccctgcctcccctgatctgttccacgggcactttagctcatgctccttgcagggcctaggaccctggtgctcggtgacttggggatgaatgagccgcagatcgagcctggccctgtggagaccaggagccaggaagtgtgggagggtagcctggagcatctctgagtgcatgagtgcgctgatctgatcgaactaagtggcttttgagctagctacagagcatcctacctagttgtacgtgcagtggggcaggaaactgagtcgtgtggcctcgagaggtccccagggcttgatgccagccagtgctagccaggtgtcaaaggctagctttaaagccatgggacaggcagagatcagagattaaatgtccaggaacggcaggtagcacttgtttccttgtggacctgtgggtcatagcatcgctgctcctttggagcttgtcttctcagattgaaacaacccagttccatttatcaggtgctgcttgctgtctgtggggttaaccagtatgagagcgccagccctggcactttaattaactctcagctatctgctgatttgagctgaccatccaacgggccactgacgtcagttttattaagcaagctggggacctcgaatccagatagctgtgtataattccaggtttgtccttcattgctggtgtcttgaggcacatcgtttaacctctctgagctttggattcctcctgtaggacacaggggtaatgtatacctacagaattaccagggagtacaattcagctcagcatatatttattgagtgtcttctatgtaacccgaatgatccaagcacggtacaagcgccaccatgagaaagcacatgctgccattaatcctatctatggatgagaaactaaagaacagaaagaacaaggatactggctcagcagcaagttgctaacagatacatctttcatcactatatcattatggtggtaagggggccacaagcaggaggatttgtatgggaggccttaagtgctgagaaaatgaaggtaggggtgcagcatggtcagccagtcagtcagtctgaaggtctgtccgtcagtcaacaaacctgagttcctactaattcaaattcagctaagagtgaatctaggctcttagagtggtgaataagactggacagttactgcctttagaattcatatcatagtgtctgtcttagtcagggtttctattgctgcaatgaaacaccataaacaaaaagcaagcttggaagaaaggatttattcagctcacatttcatatcactattcatcactgaaggaagccagaccaggaactcacacagggcaggaacctggaggcaggagctgatgcagagcctgtgaggagtgctacttactggcttgctcaccctgttttcttatagaacccaggaccttcagccctgaagtgatagcacccacaatagcctgggccttcccacattgatcactaatgaagaaaatgccttacagctggatcttatggaggagtctcttcaattaaggttcccccctttcaaatagatgtagtttgtatgtcaagtcgacataagaccagccagcacattacaagaactaagtttaaaaagaagcaaggaaggagatatggagacaatggctgaagcagtggtttgatctggtcttgcacagtctctctgaggaggtagcatctgagctgaacaaggggcaggaaacagccatatggaatgccaagaatgtcccaagctgaggggtcggcctgtgcaaaagtcctgtgacagggctgctgttgctgcgttcaggacgggaagaataccactgtggctagaacagaggaacaaagtacaaagggttacatggcttctcctggacaaccaaggagagcaagcccttggctgcaagctacaaaatgcaaagaagggcaggagagagatagatggggtggaatggggtgagaatgcactgtggcctgtagatgctgttcccttgtaactctagtttcagtgactcccaggagctgcacacacaggtgcctacttagcatctatttcttacttggagtcgttgtcagaatagtttacacacactcctggatggacctggcttttccagcctcttccaggctgcagagagctttccaagtggagggtctgagtgtattccccagtcttagcaagatcctgtgaaatctggccctggaaacaccagcttcctatgtgtctgcccggggacaaaccatgaagtgcctttgggggaaaattgtaaaattttaatgccagtacaaaccagggagctcagtgcaggctgcccaggctctgcacctcctctatgcctcctccacagttcctccgctggtacctgggagggtccttgcaggcagccacacttctctcgctgtattagttttctcagtgctgtgacaaaacacccaggagagacaacttgaggaggattttactttggctcatggcttgatgaagaagtccattatgggagggaaggcgcaacagctggagcgtgcgtggcgcagctggagcgtgcgtggcgcagctggagcgtgcgtggcgcagctggagcgtgcgtggcgcagctggagcgtgcgtggcgcagctggagcgtgcgtggcgcagctggagcgtgcgtggcgcagctggagcgtgcgtggcgcagctggagcgtgcgtggcgcagctggagcgtgcgtggcgcagctggtcgcgtcgcacccactgtcttgaagtagagactttctcatctttatgcatcccaggactccggcccatggggtagcatcacattcacagtaaggcttcccagctcagcttaacctttctcaaaacagccctatgggcatactctatagaaccacgtggtggttctcaataccatcaaattgactacaacaatcatcatgctattcctgcctctcctcccaggtcttcaagaagcctcacgacttcttcccagccaaccctctctgcccagaaactcccgatgccattcacatcgtggacctggaggttttcccaaaggtgtcactagagctgagagactcagtcctgcatggcagcaccgacagtggctttggcagtggtaaaccatcacttcagactgaagagtcccaattcctcctccctggctcccacccccagatacaggggactctgggaaaagaagagtctccagggctacaggccacctgtggggacaacacggacagtgggatctgcctgcaggagcccggcttacactccagcatggggcccgcctggaagcagcagcttggatatacccatcaggaccaggatgacagtgacgttaacctagtccagaactctccagggcagcctaagtacacacaggatgcatctgccttgggccatgtctgtctcctagaacctaaagcccctgaggagaaagaccaagtcatggtgacattccagggctaccagaaacagaccagatggaaggcagaggcagcaggcccagcagaatgcttggacgaagagattcccttgacagatgcctttgatcctgaacttggggtacacctgcaggatgatttggcttggcctccaccagctctggccgcaggttatttgaaacaggagtctcaagggatggcttctgctccaccagggacaccaagtagacagtggaatcaactgaccgaagagtggtcactcctgggtgtggttagctgtgaagatctaagcatagaaagttggaggtttgcccataaacttgaccctctggactgtggggcagcccctggtggcctcctggatagccttggctctaacctggtcaccctgccgttgatctccagcctgcaggtagaagaatgacagcggctaagagttatttgtattccagccatgcctgctcccctccctgtacctgggaggctcaggagtcaaagaaatatgtgggtccttttctgcagacctactgtgaccagctagccaggctccacggggcaaggaaaggccatcttgatacacgagtgtcaggtacatgagaggttgtggctagtctgctgagtgagggtctgtagataccagcagagctgagcaggattgacagagacctcctcatgcctcagggctggctcctacactggaaggacctgtgtttgggtgtaacctcagggctttctggatgtggtaagactgtaggtctgaagtcagctgagcctggatgtctgcggaggtgttggagtggctagcctgctacaggataaagggaaggctcaagagatagaagggcagagcatgagccaggtttaattttgtcctgtagagatggtccccagccaggatgggttacttgtggctgggagatcttggggtatacaccaccctgaatgatcagccagtcaattcagagctgtgtggcaaaagggactgagacccagaatttctgttcctcttgtgaggtgtctctgctacccatctgcagacagacatcttcatctttttactatggctgtgtcccctgaattaccagcagtggccaagccattactccctgctgctcactgttgtgacgtcagaccagaccagacgctgtctgtctgtgttagtacactaccctttaggtggcctttgggcttgagcactggcccaggcttaggacttatgtctgcttttgctgctaatctctaactgcagacccagagaacagggtgctgggctgacacctccgtgttcagctgtgtgacctccgaccagcagcttcctcaggggactaaaataatgactaggtcattcagaagtccctcatgctgaatgttaaccaaggtgcccctggggtgatagtttaggtcctgccaacctctgggttggaaggaagtggactacggaagccatctgtccccctggggagcttccacctcatgccagtgtttcagagatcttgtgggagcctagggccttgtgccaagggagctgctagtccctggggtctagggctggtccctgcctccctatactgcgtttgagacctgtcttcaaatggaggcagtttgcagcccctaagcaaggatgctgagagaagcagcaaggctgctgatccctgagcccagagtttctctgaagctttccaaatacagactgtgtgacggggtgaggccagccatgaactttggcatcctgccgagaaggtcatgaccctaatctggtacgagagctccttctggaactgggcaagctctttgagacccccctgggaacctttatttatttatttgctcacttatttattgaggaagcagcgtggcacaggcgcaaggctctgggtctctcaggaggtctagatttgcctgccctgtttctagctgtgtgaccttgggcaagtcacgtttcctcgtggagcctcagttttcctgtctgtatgcaaagcttggaaattgaaatgtacctgacgtgctccatccctaggagtgctgagtcccactgagaaagcgggcacagacgcctcaaatggaaccacaagtggtgtgtgttttcatcctaataaaaagtcaggtgttttgtggaagcgattggatttctcatggtaactgggagtgatggatgatgggtgttaacaagggccgaggacttgggccacagactcagcttcaacatgaatggtgactttggggaaggcgcatattctctcacagtttctgattccccccatggtggatgacatgggatgacagttgccatggtgatgtcatgaggttgacttcggtgtggtgccctgcatctggcattccaggtgtaaacagctgtcaagggcggttagagctggaaagggatataggctttctcttggaaacaggtgcgaatgagaaaggtgcctgggtgttctttcagaaaactaaacaacagaaagtgcagcccaatatttaggatgtttgttctgacagtgagg

SEQ ID NO: 31 - DNA, 3503 bp, Artificial Sequence
  The mRNA sequence of the engineered mouse IL-10RA after humanization
agtcttcagttctcaggacgccgccggccggaggcgtaaaggccggctccagtggacgatgccgctgtgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagttgcctacagtcctggtcttcaagaagcctcacgacttcttcccagccaaccctctctgcccagaaactcccgatgccattcacatcgtggacctggaggttttcccaaaggtgtcactagagctgagagactcagtcctgcatggcagcaccgacagtggctttggcagtggtaaaccatcacttcagactgaagagtcccaattcctcctccctggctcccacccccagatacaggggactctgggaaaagaagagtctccagggctacaggccacctgtggggacaacacggacagtgggatctgcctgcaggagcccggcttacactccagcatggggcccgcctggaagcagcagcttggatatacccatcaggaccaggatgacagtgacgttaacctagtccagaactctccagggcagcctaagtacacacaggatgcatctgccttgggccatgtctgtctcctagaacctaaagcccctgaggagaaagaccaagtcatggtgacattccagggctaccagaaacagaccagatggaaggcagaggcagcaggcccagcagaatgcttggacgaagagattcccttgacagatgcctttgatcctgaacttggggtacacctgcaggatgatttggcttggcctccaccagctctggccgcaggttatttgaaacaggagtctcaagggatggcttctgctccaccagggacaccaagtagacagtggaatcaactgaccgaagagtggtcactcctgggtgtggttagctgtgaagatctaagcatagaaagttggaggtttgcccataaacttgaccctctggactgtggggcagcccctggtggcctcctggatagccttggctctaacctggtcaccctgccgttgatctccagcctgcaggtagaagaatgacagcggctaagagttatttgtattccagccatgcctgctcccctccctgtacctgggaggctcaggagtcaaagaaatatgtgggtccttttctgcagacctactgtgaccagctagccaggctccacggggcaaggaaaggccatcttgatacacgagtgtcaggtacatgagaggttgtggctagtctgctgagtgagggtctgtagataccagcagagctgagcaggattgacagagacctcctcatgcctcagggctggctcctacactggaaggacctgtgtttgggtgtaacctcagggctttctggatgtggtaagactgtaggtctgaagtcagctgagcctggatgtctgcggaggtgttggagtggctagcctgctacaggataaagggaaggctcaagagatagaagggcagagcatgagccaggtttaattttgtcctgtagagatggtccccagccaggatgggttacttgtggctgggagatcttggggtatacaccaccctgaatgatcagccagtcaattcagagctgtgtggcaaaagggactgagacccagaatttctgttcctcttgtgaggtgtctctgctacccatctgcagacagacatcttcatctttttactatggctgtgtcccctgaattaccagcagtggccaagccattactccctgctgctcactgttgtgacgtcagaccagaccagacgctgtctgtctgtgttagtacactaccctttaggtggcctttgggcttgagcactggcccaggcttaggacttatgtctgcttttgctgctaatctctaactgcagacccagagaacagggtgctgggctgacacctccgtgttcagctgtgtgacctccgaccagcagcttcctcaggggactaaaataatgactaggtcattcagaagtccctcatgctgaatgttaaccaaggtgcccctggggtgatagtttaggtcctgccaacctctgggttggaaggaagtggactacggaagccatctgtccccctggggagcttccacctcatgccagtgtttcagagatcttgtgggagcctagggccttgtgccaagggagctgctagtccctggggtctagggctggtccctgcctccctatactgcgtttgagacctgtcttcaaatggaggcagtttgcagcccctaagcaaggatgctgagagaagcagcaaggctgctgatccctgagcccagagtttctctgaagctttccaaatacagactgtgtgacggggtgaggccagccatgaactttggcatcctgccgagaaggtcatgaccctaatctggtacgagagctccttctggaactgggcaagctctttgagacccccctgggaacctttatttatttatttgctcacttatttattgaggaagcagcgtggcacaggcgcaaggctctgggtctctcaggaggtctagatttgcctgccctgtttctagctgtgtgaccttgggcaagtcacgtttcctcgtggagcctcagttttcctgtctgtatgcaaagcttggaaattgaaatgtacctgacgtgctccatccctaggagtgctgagtcccactgagaaagcgggcacagacgcctcaaatggaaccacaagtggtgtgtgttttcatcctaataaaaagtcaggtgttttgtggaaaaaaaaaaaaa

SEQ ID NO: 32 - protein, 573 aa, Artificial Sequence
  protein sequence of the engineered mouse IL-10RA after humanization
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPTVLVFKKPHDFFPANPLCPETPDAIHIVDLEVFPKVSLELRDSVLHGSTDSGFGSGKPSLQTEESQFLLPGSHPQIQGTLGKEESPGLQATCGDNTDSGICLQEPGLHSSMGPAWKQQLGYTHQDQDDSDVNLVQNSPGQPKYTQDASALGHVCLLEPKAPEEKDQVMVTFQGYQKQTRWKAEAAGPAECLDEEIPLTDAFDPELGVHLQDDLAWPPPALAAGYLKQESQGMASAPPGTPSRQWNQLTEEWSLLGVVSCEDLSIESWRFAHKLDPLDCGAAPGGLLDSLGSNLVTLPLISSLQVEE

SEQ ID NO: 33 - DNA, 90 bp, Artificial Sequence
  The connection between the 5′ end of the Neo cassette and the mouse IL-10RA gene locus
gtaatcatagtttcatcatctacacaatggcctcgaggtcgacggtatcgataagcttgatatcgaattccgaagttcctattctctaga

SEQ ID NO: 34 - DNA, 80 bp, Artificial Sequence
  The connection between the 3 end of the Neo cassette with the mouse IL-10RA gene locus
aagtataggaacttcatcagtcaggtacataatggtggatccctgtgaaccggccactgtgtatcatgaattggtcctgg

SEQ ID NO: 35 - DNA, 25 bp, Artificial Sequence
  primer
ggtcattctcccgtaggccatgttc

SEQ ID NO: 36 - DNA, 25 bp, Artificial Sequence
  primer
ccttaccatgagcgtctgagccaag

SEQ ID NO: 37 - DNA, 20 bp, Artificial Sequence
  primer
gctcgactagagcttgcgga

SEQ ID NO: 38 - DNA, 25 bp, Artificial Sequence
  primer
ggtctttccccaacagcctttcaga

SEQ ID NO: 39 - DNA, 25 bp, Artificial Sequence
  Probe
aacccacaacagggaactgcagaag

SEQ ID NO: 40 - DNA, 25 bp, Artificial Sequence
  Probe
gttgctgggtttctagaccagagcc

SEQ ID NO: 41 - DNA, 25 bp, Artificial Sequence
  Probe
tggcctgctagaaatgcttgtgaga

SEQ ID NO: 42 - DNA, 25 bp, Artificial Sequence
  Probe
gatccccagcttccaagatactggc

SEQ ID NO: 43 - DNA, 21 bp, Artificial Sequence
  Probe
ggatcggccattgaacaagat

SEQ ID NO: 44 - DNA, 22 bp, Artificial Sequence
  Probe
cagaagaactcgtcaagaaggc

SEQ ID NO: 45 - DNA, 25 bp, Artificial Sequence
  Primer
cccagaactagctagacttcgctgc

SEQ ID NO: 46 - DNA, 25 bp, Artificial Sequence
  Primer
cagattctcccgcgaatgtgaactt

SEQ ID NO: 47 - DNA, 25 bp, Artificial Sequence
  Primer
ccttaccatgagcgtctgagccaag

SEQ ID NO: 48 - DNA, 24 bp, Artificial Sequence
  Primer
gtcctggtgagtgctctgtccttc

SEQ ID NO: 49 - DNA, 26 bp, Artificial Sequence
  Primer
gtgtgatcctgtgagtggaagcaacc

SEQ ID NO: 50 - DNA, 25 bp, Artificial Sequence
  Primer
gacaagcgttagtaggcacatatac

SEQ ID NO: 51 - DNA, 24 bp, Artificial Sequence
  Primer
gctccaatttcccacaacattagt

SEQ ID NO: 52 - DNA, 96 bp, Artificial Sequence
  The connection between the 3 end of the human IL-10 gene fragment and the mouse IL-10 gene locus
catcaactacatagaagcctacatgacaatgaagatacgaaactgaaacacctgcagtgtgtattgagtctgctggactccaggacctagacagag

SEQ ID NO: 53 - DNA, 93 bp, Artificial Sequence
  The connection between the 3 end of the human IL-10RA gene fragment and the mouse IL-10RA gene locus
ccctcgcctactgcctggccctccagctgtatgtgcggcgccgaaagaagttgcctacagtcctggtgagtgctctgtccttcctggattccc

SEQ ID NO: 54 - DNA, 22 bp, Artificial Sequence
  primer
agggcacccagtctgagaacag

SEQ ID NO: 55 - DNA, 23 bp, Artificial Sequence
  primer
gcgccttgatgtctgggtcttgg

SEQ ID NO: 56 - DNA, 27 bp, Artificial Sequence
  primer
cagctggacaacttgttgttaaaggag

SEQ ID NO: 57 - DNA, 25 bp, Artificial Sequence
  primer
actcatggctttgtagatgcctttc

SEQ ID NO: 58 - DNA, 23 bp, Artificial Sequence
  primer
tgactggcatgaggatcagcagg

SEQ ID NO: 59 - DNA, 22 bp, Artificial Sequence
  primer
ctccttgatttctgggccatgc

SEQ ID NO: 60 - DNA, 25 bp, Artificial Sequence
  primer
gctggacaacatactgctaaccgac

SEQ ID NO: 61 - DNA, 25 bp, Artificial Sequence
  primer
attcatggccttgtagacaccttgg

SEQ ID NO: 62 - DNA, 22 bp, Artificial Sequence
  primer
tcaccatcttccaggagcgaga

SEQ ID NO: 63 - DNA, 21 bp, Artificial Sequence
  primer
gaaggccatgccagtgagctt

(19) United States
(12) Patent Application Publication
(10) Pub. No.: US 2022/0378025 A1
(43) Pub. Date: Dec. 1, 2022

(54) GENETICALLY MODIFIED NON-HUMAN ANIMAL WITH HUMAN OR CHIMERIC GENES

(71) Applicant: Biocytogen Pharmaceuticals (Beijing) Co., Ltd., Daxing District, Beijing (CN)

(72) Inventors: Yuelei Shen, Beijing (CN); Yanan Guo, Beijing (CN); Yang Bai, Beijing (CN); Jiawei Yao, Beijing (CN); Chaoshe Guo, Beijing (CN); Meiling Zhang, Beijing (CN); Rui Huang, Beijing (CN); Lei Zhao, Beijing (CN); Xiaofei Zhou, Beijing (CN)

(21) Appl. No.: 17/770,727

(22) PCT Filed: Nov. 11, 2020
(86) PCT No.: PCT/CN2020/128201

§ 371 Date: Apr. 21, 2022

(30) Foreign Application Priority Data
Nov. 11, 2019 (CN) 201911095820.2

(51) Int. Cl. A01K 67/027; C07K 14/715; C07K 14/54; A61K 49/00; G01N 33/50

(52) U.S. Cl. CPC A01K 67/0278; C07K 14/7155; C07K 14/5428; A61K 49/0008; G01N 33/5088; A01K 2207/15; A01K 2217/072; A01K 2227/105; A01K 2267/0387

(57) ABSTRACT

The present disclosure relates to genetically modified non-human animals that express a human or chimeric (e.g., humanized) IL10R and/or a human or chimeric (e.g., humanized) IL10, and methods of use thereof.

### CLAIM OF PRIORITY

This application claims the benefit of Chinese Patent Application App. No. 201911095820.2, filed on Nov. 11, 2019. The entire contents of the foregoing application are incorporated herein by reference.

### TECHNICAL FIELD

This disclosure relates to genetically modified animal expressing human or chimeric (e.g., humanized) genes, and methods of use thereof.

### BACKGROUND

Interleukin-10 (IL10) is a cytokine produced by several different cell types. It acts as an anti-inflammatory cytokine that can inhibit pro-inflammatory responses of both innate and adaptive immune cells. There is substantial evidence showing that targeting IL10/IL10R pathway can be a therapeutic strategy for treating immune-related disorders (e.g., allergy and autoimmune diseases) in humans.

The traditional drug research and development for therapeutic agents that target IL10/IL10R pathway typically use in vitro screening approaches. However, these screening approaches are still different from what happens in the in vivo environment (such as cell microenvironment, extracellular matrix components and immune cell interaction, etc.), resulting in a high rate of failure in drug development. There is a need for humanized animal models that are suitable for human antibody screening and efficacy evaluation.

### SUMMARY

This disclosure is related to an animal model with human IL10R and/or IL10 or chimeric IL10R and/or IL10. The animal model can express human IL10R and/or IL10 or chimeric IL10R and/or IL10 (e.g., humanized IL10R and/or IL10) protein in its body. It can be used in the studies on the function of IL10R and/or IL10 gene, and can be used in the screening and evaluation of anti-human IL10R and anti-human IL10 antibodies. In addition, the animal models prepared by the methods described herein can be used in drug screening, pharmacodynamics studies, treatments for immune-related diseases (e.g., autoimmune disease, allergies). They can also be used to facilitate the development and design of new drugs, and save time and cost. In summary, this disclosure provides a powerful tool for studying the function of IL10R and/or IL10 protein and a platform for screening treatments for immune-related diseases.

In one aspect, the disclosure is related to a genetically-modified, non-human animal whose genome comprises at least one chromosome comprising a sequence encoding a human or chimeric interleukin-10 receptor alpha subunit (IL10RA).

In some embodiments, the sequence encoding the human or chimeric IL10RA is operably linked to an endogenous regulatory element at the endogenous IL10RA gene locus in the at least one chromosome.

In some embodiments, the sequence encoding a human or chimeric IL10RA comprises a sequence encoding an amino acid sequence that is at least 70%, 75%, 80%, 85%, 90%, 95%, 99%, or 100% identical to human IL10RA (NP_001549.2; SEQ ID NO: 28).

In some embodiments, the sequence encoding a human or chimeric IL10RA comprises a sequence encoding an amino acid sequence that is at least 70%, 75%, 80%, 85%, 90%, 95%, 99%, or 100% identical to SEQ ID NO: 32.

In some embodiments, the sequence encoding a human or chimeric IL10RA comprises a sequence that is at least 70%, 75%, 80%, 85%, 90%, 95%, 99%, or 100% identical to amino acids 1-264 of SEQ ID NO: 28.

In some embodiments, the animal is a mammal, e.g., a monkey, a rodent or a mouse.

In some embodiments, the human or chimeric IL10RA forms a functional IL10R complex with an endogenous IL10Rb.

In some embodiments, the animal does not express endogenous IL10RA.

In some embodiments, the animal has one or more cells expressing human or chimeric IL10RA.

In some embodiments, the animal has one or more cells expressing human or chimeric IL10RA, and endogenous IL10 can bind to the IL10R complex comprising the expressed human or chimeric IL10RA.

In some embodiments, the animal has one or more cells expressing human or chimeric IL10RA, and human IL10 can bind to the IL10R complex comprising the expressed human or chimeric IL10RA.

In one aspect, the disclosure is related to a genetically-modified, non-human animal, and the genome of the animal comprises a replacement of a sequence encoding a region of endogenous IL10RA with a sequence encoding a corresponding region of human IL10RA at an endogenous IL10RA gene locus.

In some embodiments, the sequence encoding the corresponding region of human IL10RA is operably linked to an endogenous regulatory element at the endogenous IL10RA locus, and one or more cells of the animal express a human or chimeric IL10RA.

In some embodiments, the animal does not express endogenous IL10RA.

In some embodiments, the replaced sequence encodes the extracellular region of IL10RA.

In some embodiments, the replaced sequence encodes the extracellular region, the transmembrane region, and a portion of the transmembrane region of IL10RA.

In some embodiments, the animal has one or more cells expressing a chimeric IL10RA having an extracellular region. In some embodiments, the extracellular region comprises a sequence that is at least 50%, 60%, 70%, 80%, 90%, 95%, or 99% identical to the extracellular region of human IL10RA.

In some embodiments, the extracellular region of the chimeric IL10RA has a sequence that has at least 10, 20, 30, 40, 50, 60, 70, 80, 90, 100, or 150 contiguous amino acids that are identical to a contiguous sequence present in the extracellular region of human IL10RA.

In some embodiments, the animal is a mouse, and the replaced endogenous IL10RA region is a portion of exon 1, exon 2, exon 3, exon 4, exon 5, and/or a portion of exon 6 of the endogenous mouse IL10RA gene.

In some embodiments, the animal is heterozygous with respect to the insertion at the endogenous IL10RA gene locus. In some embodiments, the animal is homozygous with respect to the insertion at the endogenous IL10RA gene locus.

In one aspect, the disclosure is related to a method for making a genetically-modified, non-human animal, comprising: replacing in at least one cell of the animal, at an endogenous IL10RA gene locus, a sequence encoding a region of an endogenous IL10RA with a sequence encoding a corresponding region of human IL10RA.

In some embodiments, the sequence encoding the corresponding region of human IL10RA comprises a portion of exon 1, exon 2, exon 3, exon 4, exon 5, and/or a portion of exon 6 of a human IL10RA gene.

In some embodiments, the sequence encoding the corresponding region of human IL10RA comprises at least 50, 100, 200, or 300 nucleotides of exon 1, exon 2, exon 3, exon 4, exon 5, and/or exon 6 of a human IL10RA gene.

In some embodiments, the sequence encoding the corresponding region of human IL10RA encodes a sequence that is at least 90% identical to amino acids 1-264 of SEQ ID NO: 28.

In some embodiments, the locus is located at the extracellular region of IL10RA.

In some embodiments, the locus comprises the sequence encodes the extracellular region and the transmembrane region of IL10RA.

In some embodiments, the animal is a mouse, and the locus is a portion of exon 1, exon 2, exon 3, exons 4, exon 5, and/or a portion of exon 6 of the mouse IL10RA gene.

In one aspect, the disclosure is related to a non-human animal comprising at least one cell comprising a nucleotide sequence encoding a chimeric IL10RA polypeptide. In some embodiments, the chimeric IL10RA polypeptide comprises at least 50 contiguous amino acid residues that are identical to the corresponding contiguous amino acid sequence of a human IL10RA. In some embodiments, the animal expresses the chimeric IL10RA.

In some embodiments, the chimeric IL10RA polypeptide has at least 50 contiguous amino acid residues that are identical to the corresponding contiguous amino acid sequence of a human IL10RA extracellular region.

In some embodiments, the chimeric IL10RA polypeptide comprises a sequence that is at least 90%, 95%, or 99% identical of SEQ ID NO: 32.

In some embodiments, the nucleotide sequence is operably linked to an endogenous IL10RA regulatory element of the animal.

In some embodiments, the nucleotide sequence is integrated to an endogenous IL10RA gene locus of the animal.

In some embodiments, the chimeric IL10RA has at least one mouse IL10RA activity and/or at least one human IL10RA activity.

In one aspect, the disclosure is related to a method of making a genetically-modified mouse cell that expresses a chimeric IL10RA, the method comprising: replacing, at an endogenous mouse IL10RA gene locus, a nucleotide sequence encoding a region of mouse IL10RA with a nucleotide sequence encoding a corresponding region of human IL10RA, thereby generating a genetically-modified mouse cell that includes a nucleotide sequence that encodes the chimeric IL10RA. In some embodiments, the mouse cell expresses the chimeric IL10RA.

In some embodiments, the chimeric IL10RA comprises: an extracellular region of human IL10RA; a transmembrane region of human IL10RA; and/or a cytoplasmic region that is at least 90% identical to mouse IL10RA cytoplasmic region.

In some embodiments, the nucleotide sequence encoding the chimeric IL10RA is operably linked to an endogenous IL10RA regulatory region, e.g., promoter.

In some embodiments, the animal further comprises a sequence encoding an additional human or chimeric protein (e.g., IL10, IL3, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal regulatory protein α (SIRPα)).

In some embodiments, the additional human or chimeric protein is IL10.

In some embodiments, the animal or mouse further comprises a sequence encoding an additional human or chimeric protein (e.g., IL10, IL3, PD-1, CTLA-4, LAG-3, BTLA, PD-L1, CD27, CD28, TIGIT, TIM-3, GITR, CD137, OX40, CD47 or SIRPa). In some embodiments, the additional human or chimeric protein is IL10.

In one aspect, the disclosure is related to a genetically-modified, non-human animal whose genome comprises at least one chromosome comprising a sequence encoding a human or chimeric IL10.

In some embodiments, the sequence encoding the human or chimeric IL10 is operably linked to an endogenous regulatory element at the endogenous IL10 gene locus in the at least one chromosome.

In some embodiments, the sequence encoding a human or chimeric IL10 comprises a sequence encoding an amino acid sequence that is at least 70%, 75%, 80%, 85%, 90%, 95%, 99%, or 100% identical to human IL10 (NP_000563.1; SEQ ID NO: 4).

In some embodiments, the animal comprises a sequence that is at least 70%, 75%, 80%, 85%, 90%, 95%, 99%, or 100% identical to SEQ ID NO: 7.

In some embodiments, the animal comprises a sequence that is at least 70%, 75%, 80%, 85%, 90%, 95%, 99%, or 100% identical to SEQ ID NO: 52.

In some embodiments, the animal comprises a sequence that is at least 70%, 75%, 80%, 85%, 90%, 95%, 99%, or 100% identical to SEQ ID NO: 8 or SEQ ID NO: 9.

In some embodiments, the animal is a mammal, e.g., a monkey, a rodent or a mouse. In some embodiments, the animal is a mouse.

In some embodiments, the animal does not express endogenous IL10, or expresses a decreased level of endogenous IL10 as compared to IL10 expression level in a wild-type animal. In some embodiments, the animal has one or more cells expressing human IL10.

In some embodiments, the animal has one or more cells expressing human or chimeric IL10, and the expressed human or chimeric IL10 can bind to endogenous IL10R complex.

In some embodiments, the animal has one or more cells expressing human or chimeric IL10, and the expressed human or chimeric IL10 can bind to human IL10R complex.

In one aspect, the disclosure is related to a genetically-modified, non-human animal, and the genome of the animal comprises a replacement of a sequence encoding a region of endogenous IL10 with a sequence encoding a corresponding region of human IL10 at an endogenous IL10 gene locus.

In some embodiments, the sequence encoding the corresponding region of human IL10 is operably linked to an endogenous regulatory element at the endogenous IL locus, and one or more cells of the animal expresses a human IL10.

In some embodiments, the animal does not express endogenous IL10, or expresses a decreased level of endogenous IL10 as compared to IL10 expression level in a wild-type animal.

In some embodiments, the replaced locus comprises a sequence that is at least 70%, 75%, 80%, 85%, 90%, 95%, 99%, or 100% identical to SEQ ID NO: 7, SEQ ID NO: 8, SEQ ID NO: 9, or SEQ ID NO: 52.

In some embodiments, the animal is a mouse, and the replaced endogenous IL10 region is a portion of exon 1, exon 2, exon 3, exon 4 and/or a portion of exon 5 of the endogenous mouse IL10 gene.

In some embodiments, the animal is heterozygous with respect to the replacement at the endogenous IL10 gene locus. In some embodiments, the animal is homozygous with respect to the replacement at the endogenous IL10 gene locus.

In one aspect, the disclosure is related to a method for making a genetically-modified, non-human animal, comprising: replacing in at least one cell of the animal, at an endogenous IL10 gene locus, a sequence encoding a region of an endogenous IL10 with a sequence encoding a corresponding region of human IL10.

In some embodiments, the sequence encoding the corresponding region of human IL10 comprises a portion of exon 1, exon 2, exon 3, exon 4, and/or a portion of exon 5 of a human IL10 gene.

In some embodiments, the sequence encoding the corresponding region of IL10 comprises at least 30, 50, 75, 100, or 150 nucleotides of exon 1, exon 2, exon 3, exon 4, and/or exon 5 of a human IL10 gene.

In some embodiments, the sequence encoding the corresponding region of human IL10 encodes a sequence that is at least 90% identical to SEQ ID NO: 4.

In some embodiments, replaced locus comprises a sequence of SEQ ID NO: 7, SEQ ID NO: 8, SEQ ID NO: 9, or SEQ ID NO: 52.

In some embodiments, the animal is a mouse, and the locus is a portion of exon 1, exon 2, exon 3, exon 4, and a portion of exon 5 of the mouse IL10 gene.

In one aspect, the disclosure is related to a non-human animal comprising at least one cell comprising a nucleotide sequence encoding a human or chimeric IL10 polypeptide. In some embodiments, the human or chimeric IL10 polypeptide comprises at least 50 contiguous amino acid residues that are identical to the corresponding contiguous amino acid sequence of a human IL10. In some embodiments, the animal expresses the human or chimeric IL10.

In some embodiments, the human or chimeric IL10 polypeptide has at least 100 contiguous amino acid residues that are identical to the corresponding contiguous amino acid sequence of a human IL10.

In some embodiments, the nucleotide sequence is operably linked to an endogenous IL10 regulatory element of the animal.

In some embodiments, the nucleotide sequence is integrated to an endogenous IL10 gene locus of the animal.

In one aspect, the disclosure is related to a method of making a genetically-modified mouse cell that expresses a human or chimeric IL10, the method comprising: replacing, at an endogenous mouse IL10 gene locus, a nucleotide sequence encoding a region of mouse IL10 with a nucleotide sequence encoding a corresponding region of human IL10, thereby generating a genetically-modified mouse cell that includes a nucleotide sequence that encodes the human or chimeric IL10. In some embodiments, the mouse cell expresses the chimeric IL10.

In some embodiments, the nucleotide sequence encoding the human or chimeric IL10 is operably linked to an endogenous IL10 regulatory region, e.g., promoter.

In some embodiments, the animal further comprises a sequence encoding an additional human or chimeric protein (e.g., IL10R, IL10RA, IL10Rb, IL3, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or SIRPa). In some embodiments, the additional human or chimeric protein is IL10RA.

In some embodiments, the animal or mouse further comprises a sequence encoding an additional human or chimeric protein (e.g., IL10R, IL10RA, IL10Rb, IL3, PD-1, CTLA-4, LAG-3, BTLA, PD-L1, CD27, CD28, TIGIT, TIM-3, GITR, CD137, OX40, CD47, or SIRPa). In some embodiments, the additional human or chimeric protein is IL10RA.

In one aspect, the disclosure is related to a method of determining effectiveness of an IL10/IL10R pathway modulator for treating an autoimmune disorder, comprising: administering the IL10/IL10R pathway modulator to the animal as described herein; and determining the effects of the IL10/IL10R pathway modulator.

In some embodiments, the IL10/IL10R pathway modulator is an anti-human IL10 antibody. In some embodiments, the IL10/IL10R pathway modulator is an anti-human IL10R antibody.

In some embodiments, the autoimmune disorder is asthma, systemic lupus erythematosus (SLE), rheumatoid arthritis (RA), Sjogren's syndrome (SS), multiple sclerosis (MS), Crohn's disease (CD), inflammatory bowel disease (IBD), or psoriasis.

In one aspect, the disclosure is related to a method of determining effectiveness of an IL10/IL10R pathway modulator for reducing inflammation, comprising: administering the IL10/IL10R pathway modulator to the animal as described herein; and determining the effects of the IL10/IL10R pathway modulator.

In one aspect, the disclosure is related to a method of determining effectiveness of an IL10/IL10R pathway modulator for treating cancer, comprising: administering the IL10/IL10R pathway modulator to the animal as described herein; and determining the effects of the IL10/IL10R pathway modulator.

In some embodiments, the cancer is a solid tumor, colorectal cancer, melanoma, or pancreatic cancer.

In one aspect, the disclosure is related to a method of determining effectiveness of an IL10/IL10R pathway modulator for treating an infectious disease (e.g., tuberculosis), comprising: administering the IL10/IL10R pathway modulator to the animal as described herein; and determining the effects of the IL10/IL10R pathway modulator.

In one aspect, the disclosure is related to a method of determining toxicity of an anti-IL10R antibody or an anti-IL10 antibody, the method comprising administering the anti-IL10R antibody or the anti-IL10 antibody to the animal as described herein; and determining weight change of the animal.

In some embodiments, the method further comprises performing a blood test (e.g., determining red blood cell count).

In one aspect, the disclosure is related to a protein comprising an amino acid sequence, and the amino acid sequence is one of the following:
(a) an amino acid sequence set forth in SEQ ID NO: 32;
(b) an amino acid sequence that is at least 90%, 91%, 92%, 93%, 94%, 95%, 96%, 97%, 98%, or 99% identical to SEQ ID NO: 32;
(c) an amino acid sequence that is different from the amino acid sequence set forth in SEQ ID NO: 32 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 32.

In one aspect, the disclosure is related to a nucleic acid comprising a nucleotide sequence, and the nucleotide sequence is one of the following:
(a) a sequence that encodes the protein as described herein;
(b) SEQ ID NO: 7, 8, 9, 31, 33, 34, 52, or 53; or
(c) a sequence that is at least 90%, 91%, 92%, 93%, 94%, 95%, 96%, 97%, 98%, or 99% identical to SEQ ID NO: 7, 8, 9, 31, 33, 34, 52, or 53.

In one aspect, the disclosure is related to a cell comprising the protein as described herein and/or the nucleic acid as described herein.

In one aspect, the disclosure is related to an animal comprising the protein as described herein and/or the nucleic acid as described herein.

The disclosure also relates to a method for establishing a genetically-modified non-human animal expressing two human or chimeric (e.g., humanized) genes. The method includes the steps of (a) using the method for establishing a IL10RA gene humanized animal model to obtain a IL10RA gene genetically modified humanized mouse; (b) mating the IL10RA gene genetically modified humanized mouse obtained in step (a) with another humanized mouse, and then screening to obtain a double humanized mouse model. In some embodiments, in step (b), the IL10RA gene genetically modified humanized mouse obtained in step (a) is mated with an IL10 humanized mouse to obtain a IL10RA and IL10 double humanized mouse model.

The disclosure also relates to a method for establishing a genetically-modified non-human animal expressing two human or chimeric (e.g., humanized) genes. The method includes the steps of (a) using the method for establishing a IL10 gene humanized animal model to obtain a IL10 gene genetically modified humanized mouse; (b) mating the IL10 gene genetically modified humanized mouse obtained in step (a) with another humanized mouse, and then screening to obtain a double humanized mouse model. In some embodiments, in step (b), the IL10 gene genetically modified humanized mouse obtained in step (a) is mated with an IL10RA humanized mouse to obtain an IL10 and IL10RA double humanized mouse model.

The disclosure also relates to non-human mammal generated through the methods as described herein. In some embodiments, the genome thereof contains human gene(s).

In some embodiments, the non-human mammal is a rodent. In some embodiments, the non-human mammal is a mouse. In some embodiments, the non-human mammal expresses human IL10RA and/or human IL10.

The disclosure also relates to an offspring of the non-human mammal.

In one aspect, the disclosure relates to a non-human mammal model, characterized in that the non-human mammal model is obtained through the methods as described herein. In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal. The disclosure further relates to the tissue, organ or a culture thereof derived from the non-human mammal or an offspring thereof, or the tumor bearing non-human mammal.

In one aspect, the disclosure relates to a tumor tissue derived from the non-human mammal or an offspring thereof when it bears a tumor, or the tumor bearing non-human mammal.

The disclosure further relates to a IL10R (e.g., IL10RA) and/or IL10 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the non-human mammal, the animal model generated through the method as described herein in the production and utilization of an animal experimental disease model of immunization processes involving human cells, the study on a pathogen, or the development of a new diagnostic strategy and/or a therapeutic strategy.

The disclosure further relates to the use of the non-human mammal or an offspring thereof, or the non-human mammal, the animal model generated through the methods as described herein, in the screening, verifying, evaluating or studying the IL10R and/or IL10 gene function, human IL10R and/or IL10 antibodies, the drugs or efficacies for human IL10R and/or IL10 targeting sites, and the drugs for immune-related diseases.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse IL-10 gene locus.

FIG. 1B is a schematic diagram showing human IL-10 gene locus.

FIG. 2 is a schematic diagram showing humanized IL-10 gene locus.

FIG. 3 is a schematic diagram showing an IL-10 gene targeting strategy.

FIG. 4 shows Southern Blot results. WT is wild-type.

FIG. 5 is a schematic diagram showing the FRT recombination process. Positive heterozygous mice were mated with the Flp mice.

FIG. 6A shows PCR identification result of samples collected from tails of F1 generation mice. Primers IL-10-WT-F/IL-10-WT-R were used for amplification. WT is wild-type C57BL/6 mice. H₂O is a blank control. PC is a positive control. M is marker.

FIG. 6B shows PCR identification result of samples collected from tails of F1 generation mice. Primers IL-10-WT-F/IL-10-Mut-R were used for amplification. WT is wild-type C57BL/6 mice. H₂O is a blank control. PC is a positive control. M is marker.

FIG. 7A shows mouse IL-10 level in mouse serum, as determined by ELISA. +/+ represents wild-type C57BL/6 mice and H/H represents IL-10 gene humanized homozygous mice.

FIG. 7B shows mouse IL-10 level in mouse serum, as determined by ELISA. +/+ represents wild-type C57BL/6 mice and H/H represents IL-10 gene humanized homozygous mice.

FIGS. 7C-7D are IL-10 expression results from RT-PCR.

FIG. 8A is a schematic diagram showing mouse IL-10RA gene locus.

FIG. 8B is a schematic diagram showing human IL-10RA gene locus.

FIG. 9 is a schematic diagram showing humanized IL-10RA gene locus.

FIG. 10 is a schematic diagram showing an IL-10RA gene targeting strategy.

FIG. 11A shows Southern Blot results using the IL10RA-5′ Probe. WT is wild-type.

FIG. 11B shows Southern Blot results using the IL10RA-3′ Probe. WT is wild-type.

FIG. 11C shows Southern Blot results using the IL10RA-Neo Probe. WT is wild-type.

FIG. 12A shows PCR identification result of samples collected from tails of F1 generation mice. Primers IL10RA-WT-F/IL10RA-Mut-R were used for amplification. WT is wild-type C57BL/6 mice. H₂O is a blank control. PC is a positive control. M is marker.

FIG. 12B shows PCR identification result of samples collected from tails of F1 generation mice. Primers IL10RA-WT-F/IL10RA-WT-R1 were used for amplification. WT is wild-type C57BL/6 mice. H₂O is a blank control. PC is a positive control. M is marker.

FIG. 12C shows PCR identification result of samples collected from tails of F1 generation mice. Primers Flp-F2/Flp-R2 were used for amplification. WT is wild-type C57BL/6 mice. H₂O is a blank control. PC is a positive control. M is marker.

FIG. 12D shows PCR identification result of samples collected from tails of F1 generation mice. Primers IL10RA-Frt-F/IL10RA-Frt-R were used for amplification. WT is wild-type C57BL/6 mice. H₂O is a blank control. PC is a positive control. M is marker.

FIG. 13 shows percentage of immune cells in CD45+ cells from mouse spleen. C57BL/6 represents wild-type C57BL/6 mice and H/H represents IL-10 gene humanized homozygous mice. The immune cells include B cells, T cells, NK cells, CD4+ T cells (CD4), CD8+ T cells (CD8), granulocytes, DC cells, macrophages, and monocytes.

FIG. 14 shows percentage of immune cells in CD45+ cells from mouse peripheral blood. C57BL/6 represents wild-type C57BL/6 mice and H/H represents IL-10 gene humanized homozygous mice. The immune cells include B cells, T cells, NK cells, CD4+ T cells (CD4), CD8+ T cells (CD8), granulocytes, DC cells, macrophages, and monocytes.

FIG. 15 shows percentage of TCRβ+ cells from mouse spleen. C57BL/6 represents wild-type C57BL/6 mice and H/H represents IL-10 gene humanized homozygous mice. The T cell subsets include CD4+ T cells (CD4), CD8+ T cells (CD8), and Treg cells.

FIG. 16 shows percentage of TCRβ+ cells from mouse peripheral blood. C57BL/6 represents wild-type C57BL/6 mice and H/H represents IL-10 gene humanized homozygous mice. The T cell subsets include CD4+ T cells (CD4), CD8+ T cells (CD8), and Treg cells.

FIG. 17 shows amino acid sequence alignment result between mouse IL10 protein (NP_034678.1; SEQ ID NO: 2) and human IL10 protein (NP_000563.1; SEQ ID NO: 4).

FIG. 18 shows amino acid sequence alignment result between mouse IL10RA protein (NP_032374.1; SEQ ID NO: 26) and human IL10RA protein (NP_001549.2; SEQ ID NO: 28).

### DETAILED DESCRIPTION

This disclosure relates to transgenic non-human animal with human or chimeric (e.g., humanized) IL10R and/or IL10, and methods of use thereof.

IL-10 (Interleukin-10) is a pleiotropic cytokine with important immunoregulatory functions. Its actions influence activities of many of the cell-types in the immune system. It is a cytokine with potent anti-inflammatory properties, repressing the expression of inflammatory cytokines such as TNF-Alpha (Tumor Necrosis Factor-Alpha), IL-6 (Interleukin-6) and IL-1 (Interleukin-1) by activated macrophages. Functional IL-10R (IL-10 Receptor) complexes are tetramers consisting of two ligand-binding subunits (IL-10Ra or IL-10R1) and two accessory signaling subunits (IL-10Rb or IL-10R2). Binding of IL-10 to the extracellular domain of IL-10Ra activates phosphorylation of the receptor-associated, JAK1 (Janus Kinase-1) and TYK2 (Tyrosine Kinase-2), which are constitutively associated with IL-10Ra and IL-10Rb, respectively. These kinases then phosphorylate specific tyrosine residues (Y446 and Y496) on the intracellular domain of the IL-10Ra chain. Once phosphorylated, these tyrosine residues (and their flanking peptide sequences) serve as temporary docking sites for the latent transcription factor, STAT3 (Signal Transducer and Activator of Transcription-3). STAT3 binds to these sites via its SH2 (Src Homology-2) domain, and is, in turn, tyrosine-phosphorylated by the receptor-associated JAKs. It then homodimerizes and translocates to the nucleus where it binds with high affinity to SBE (STAT-Binding Elements) in the promoters of various IL-10-responsive genes. Constitutively active forms of STAT3 increase transcription of anti-apoptotic and cell-cycle-progression genes such as BCLXL, Cyclin-D1, Cyclin-D2, Cyclin-D3, and Cyclin-A, Pim1, c-Myc, and p19 (INK4D).

As currently recognized inflammatory and immunosuppressive factors, IL-10 and IL-10RA are widely used in disease research. Studies have shown that IL10 and IL10RA play an important role in cancer development, infections, organ transplantation, hematopoietic system and cardiovascular system, and they are also closely related to blood, digestion, especially cardiovascular diseases. For example, studies have shown that the expression of IL-10 in the respiratory tract of patients with asthma and allergic rhinitis is lower than that in healthy subjects. IL-10 knockout mice can produce chronic colitis. IL-10 expression is increased in colorectal cancer and adjacent tissues. Intestinal flora disorders in children with very early-onset inflammatory bowel disease (VEO-IBD) can be detected due to functional defects of IL-10RA gene. IL-10RA polymorphism is related to the pathogenesis of systemic lupus erythematosus, etc. Based on the important functions of IL-10 and its receptor, a variety of drugs targeting IL10/IL10R pathway have entered the clinic trial. For example, Merck's MK-1966 combined with TLR9 antagonist for the treatment of malignant tumors (NCT02731742); Biotest AG's BT-063 combined with PD-1 antibody for the treatment of melanoma (WO2019072566), etc. In addition, there are also adenovirus vectors or PEGylated IL-10 drugs for tumor treatment, such as Pegilodecakin jointly developed by ARMO BioSciences and Merck. Thus, antibodies against IL10/IL10R pathway can be potentially used for treating autoimmune diseases, infectious diseases, and cancers.

Experimental animal models are an indispensable research tool for studying the effects of these antibodies before clinical trials. Common experimental animals include mice, rats, guinea pigs, hamsters, rabbits, dogs, monkeys, pigs, fish and so on. However, there are differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels. Furthermore, because of interaction between human IL10R and human IL10, a desirable animal model for the investigation of anti-IL10R or anti-IL10 antibodies should faithfully mimic the interaction between human IL10R and human IL10, elicit robust responses from both the innate and adaptive immunity, and recapitulate side effects of IL10 blockade in human patients.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U. S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### Interleukin 10 (IL10)

Interleukin-10 (IL-10, or IL10), also known as “cytokine synthesis inhibitory factor” (CSIF), is a pleiotropic cytokine with important immunoregulatory functions. IL10 is the foremost member of the type-II cytokine family, comprising IL19, IL20, IL22, IL24, IL26, IL28, and IL29. It has anti-inflammatory properties and influences the activity of several cell types of the immune system. IL-10 is primarily secreted by activated T-cells, monocytes, macrophages, dendritic cells, natural killer (NK) cells and B-cells. It is released upon activation of these cells by endogenous and exogenous mediators such as lipopolysaccharides, catecholamines and cAMP-elevating drugs. In response to antigens, cells of the immune system produce pro-inflammatory cytokines such as tumor necrosis factor alpha (TNF-α), interferon-gamma (IFN-γ), interleukin-2 (IL-2) and interleukin-1 (IL-1). They are involved in rapid pathogen clearance and cell necrosis at the site of infection. Prolonged action of pro-inflammatory cytokines can lead to excessive tissue damage, fever, inflammation, and death in extreme cases. IL-10 dampens the inflammatory effects of pro-inflammatory cytokines in overwhelming infections which otherwise can lead to potential tissue damage.

The human IL10 gene is about 4.7 kb in size and is located on the long arm of chromosome 1. It contains five exons. Biologically functional IL-10 exists in the form of a 36 kD homodimer composed of two non-covalently bonded monomers. Two disulfide bridges exist between the monomers, which are required for their biological activity and maintaining structural integrity. IL-10 homodimer binds to tetrameric IL-10 receptor complex, which consists of two IL-10Ra and two IL-10Rb subunits. IL-10Ra is known to bind to the ligand and IL-10Rb is the accessory signaling subunit.

IL-10 homodimer upon binding to its receptor IL-10Ra, activates JAK/STAT (Janus kinase/signal transducer and activator of transcription) and Akt (also known as protein kinase B, PKB) cascades. IL-10 has also been involved in proliferation, survival and anti-apoptotic activities of several cancers such as Burkitt lymphoma, non-Hodgkins lymphoma and non-small cell lung cancer. Studies have reported that overexpression of IL-10 promotes tumor development in certain lymphomas and melanomas by suppressing the antitumor immune response. Several investigations have also suggested that serum level of IL-10 may indicate disease progression. A study in advanced solid tumors has reported that IL-10 serum level returns to normal in radically resected patients. However, in case of tumor recurrence, the IL-10 level was observed to be persistently elevated. Additionally, elevated serum level of IL-10 has also been associated with autoimmune and inflammatory diseases such as systemic lupus erythematosus (SLE), systemic sclerosis, and Bullous pemphigoid. Studies have reported a protective role of IL-10 in cartilage of osteoarthritis patients. Recombinant human IL-10 has also been tested as a promising therapeutic agent in patients with rheumatoid arthritis and Crohn's disease.

IL-10 plays a central role in survival and persistence of intracellular pathogens in vivo. Pathogens such as Leishmania donovani, Mycobacterium tuberculosis, Trypanosoma cruzi, and Coxiella burnetii have evolved various mechanisms for stimulating IL-10 production in immune cells for their survival. Studies have indicated the correlation between IL-10 and tuberculosis (TB) susceptibility in humans and mice. IL-10 mediated deactivation of macrophages leads to reduced production of pro-inflammatory cytokines and reactive oxygen species which would otherwise lead to cell damage. During Mycobacterium tuberculosis infection, IL-10 contributes to survival and persistence of bacteria inside macrophage by inhibiting MHC-restricted cytotoxicity against infected macrophages. The abundance of IL-10 in TB infected individuals, and its role in downregulation of IFN-gamma and a T-cell costimulatory molecule called cytotoxic T-lymphocyte-associated protein 4 (CTLA4), suggest an altered antigen presentation in infected individuals. In addition, the CD8+ CTL-mediated cytotoxic activity could be enhanced upon neutralization of IL-10, suggesting a direct role of IL-10 in suppressing the anti-mycobacterial response.

A detailed description of IL10 and its function can be found, e.g., in Shouval, D. S., et al. “Interleukin 10 receptor signaling: master regulator of intestinal mucosal homeostasis in mice and humans.” Advances in Immunology. Vol. 122. Academic Press, 2014. 177-210; Verma, R., et al. “A network map of Interleukin-10 signaling pathway.” Journal of cell communication and signaling 10.1 (2016): 61-67; each of which is incorporated by reference herein in the entirety.

In human genomes, IL10 gene (Gene ID: 3586) locus has 5 exons, exon 1, exon 2, exon 3, exon 4, and exon 5. The nucleotide sequence for human IL10 mRNA is NM 000572.3 (SEQ ID NO: 3), and the amino acid sequence for human IL10 is NP_000563.1 (SEQ ID NO: 4). The location for each exon and each region in human IL10 nucleotide sequence and amino acid sequence is listed below:

TABLE 1

| | NM_000572.3 | NP_000563.1 |
|---|---|---|
| Human IL10 | 1630 bp | 178 aa |
| (approximate location) | (SEQ ID NO: 3) | (SEQ ID NO: 4) |
| Exon 1 | 1-224 | 1-55 |
| Exon 2 | 225-284 | 56-75 |
| Exon 3 | 285-437 | 76-126 |
| Exon 4 | 438-503 | 127-148 |
| Exon 5 | 504-1630 | 149-178 |
| Signal peptide | 60-113 | 1-18 |
| Donor region in Example | 60-596 | 1-178 |

In mice, IL10 gene locus has 5 exons, exon 1, exon 2, exon 3, exon 4, and exon 5. The nucleotide sequence for mouse IL10 mRNA is NM_010548.2 (SEQ ID NO: 1), the amino acid sequence for mouse IL10 is NP_034678.1 (SEQ ID NO: 2). The location for each exon and each region in the mouse IL10 nucleotide sequence and amino acid sequence is listed below:

TABLE 2

| | NM_010548.2 | NP_034678.1 |
|---|---|---|
| Mouse IL10 | 1306 bp | 178 aa |
| (approximate location) | (SEQ ID NO: 1) | (SEQ ID NO: 2) |
| Exon 1 | 1-232 | 1-55 |
| Exon 2 | 233-292 | 56-75 |
| Exon 3 | 293-445 | 76-126 |
| Exon 4 | 446-511 | 127-148 |
| Exon 5 | 512-1306 | 149-178 |
| Signal peptide | 68-121 | 1-18 |
| Replaced region in Example | 68-537 | 1-178 |

The mouse IL10 gene (Gene ID: 16153) is located in Chromosome 1 of the mouse genome, which is located from 131,019,845 to 131,024,974 of NC_000067.6 (GRCm38: CM00099 4.2). The 5′-UTR is from 131,019,845 to 131,019,911, exon 1 is from 131,019,845 to 131,020,076, the first intron is from 131,020,077 to 131,020,963, exon 2 is from 131,020,964 to 131,021,023, the second intron is from 131,021,024 to 131,021,328, exon 3 is from 131,021,329 to 131,021,481, the third intron is from 131,021,482 to 131,022,488, exon 4 is from 131,022,489 to 131,022,554, the fourth intron is from 131,022,555 to 131,024,175, exon 5 is from 131,024,176 to 131,024,974, the 3′-UTR is from 131,024,269 to 131,024,974, based on transcript NM_010548.2. All relevant information for mouse IL locus can be found in the NCBI website with Gene ID: 16153, which is incorporated by reference herein in its entirety.

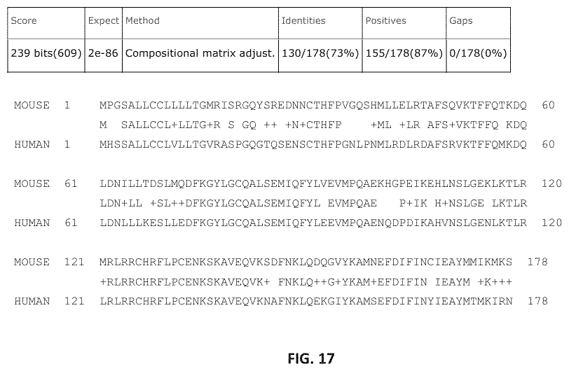

FIG. 17 shows the alignment between mouse IL10 amino acid sequence (NP_034678.1; SEQ ID NO: 2) and human IL10 amino acid sequence (NP_000563.1; SEQ ID NO: 4). Thus, the corresponding amino acid residue or region between human and mouse IL10 can also be found in FIG. 17.

IL10 genes, proteins, and locus of the other species are also known in the art. For example, the gene ID for IL10 in Rattus norvegicus is 25325, the gene ID for IL10 in Macaca mulatta (Rhesus monkey) is 694931, the gene ID for IL10 in Sus scrofa (pig) is 397106, and the gene ID for IL10 in Canis lupus familiaris (dog) is 403628. The relevant information for these genes (e.g., intron sequences, exon sequences, amino acid residues of these proteins) can be found, e.g., in NCBI database, which are incorporated herein by reference in the entirety.

The present disclosure provides human or chimeric (e.g., humanized) IL10 nucleotide sequence and/or amino acid sequences. In some embodiments, the entire sequence of mouse signal peptide, exon 1, exon 2, exon 3, exon 4, and/or exon 5, are replaced by the corresponding human sequence.

In some embodiments, a “region” or “portion” of mouse signal peptide, exon 1, exon 2, exon 3, exon 4, and/or exon 5 is replaced by the corresponding human sequence.

In some embodiments, the “region” or “portion” can be at least 50%, 55%, 60%, 65%, 70%, 75%, 80%, 85%, 90%, 95%, or 99% identical to signal peptide, exon 1, exon 2, exon 3, exon 4, and/or exon 5. In some embodiments, a region, a portion, or the entire sequence of mouse signal peptide, exon 1, exon 2, exon 3, exon 4, and/or exon 5 is replaced by a region, a portion, or the entire sequence of human signal peptide, exon 1, exon 2, exon 3, exon 4, and/or exon 5.

In some embodiments, a “region” or “portion” of mouse signal peptide, exon 1, exon 2, exon 3, exon 4, and/or exon 5 is deleted.

In some embodiments, the present disclosure is related to a genetically-modified, non-human animal whose genome comprises a chimeric (e.g., humanized) IL10 nucleotide sequence. In some embodiments, the chimeric (e.g., humanized) IL10 nucleotide sequence encodes a IL10 protein comprising a signal peptide. In some embodiments, the signal peptide described herein is at least 80%, 85%, 90%, 95%, or 100% identical to amino acids 1-18 of SEQ ID NO: 2. In some embodiments, the signal peptide described herein is at least 80%, 85%, 90%, 95%, or 100% identical to amino acids 1-18 of SEQ ID NO: 4. In some embodiments, the genome of the animal comprises a sequence that is at least 80%, 85%, 90%, 95%, or 100% identical to SEQ ID NO: 7.

Thus, in some embodiments, the present disclosure also provides a chimeric (e.g., humanized) IL10 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL10 mRNA sequence (e.g., SEQ ID NO: 1), mouse IL10 amino acid sequence (e.g., SEQ ID NO: 2), or a portion thereof (e.g., exon 1, exon 2, exon 3, exon 4, and/or exon 5); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL10 mRNA sequence (e.g., SEQ ID NO: 3), human IL10 amino acid sequence (e.g., SEQ ID NO: 4), or a portion thereof (e.g., exon 1, exon 2, exon 3, exon 4, and/or exon 5).

In some embodiments, the sequence encoding full-length amino acid sequence of mouse IL10 (SEQ ID NO: 2) is replaced. In some embodiments, the sequence is replaced by a sequence encoding a corresponding region of human IL10 (e.g., full-length amino acid sequence of human IL10 (SEQ ID NO: 4)).

In some embodiments, the sequence encoding amino acids 1-178 of mouse IL10 (SEQ ID NO: 2) is replaced. In some embodiments, the sequence is replaced by a sequence encoding a corresponding region of human IL10 (e.g., amino acids 1-178 of human IL10 (SEQ ID NO: 4)).

In some embodiments, the sequence encoding amino acids 19-178 of mouse IL10 (SEQ ID NO: 2) is replaced. In some embodiments, the sequence is replaced by a sequence encoding a corresponding region of human IL10 (e.g., amino acids 19-178 of human IL10 (SEQ ID NO: 4)).

In some embodiments, the nucleic acids as described herein are operably linked to a promotor or regulatory element, e.g., an endogenous mouse IL10 promotor, a human IL10 promotor, an inducible promoter, a human enhancer, a mouse enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, or 60 nucleotides, e.g., contiguous or non-contiguous nucleotides) that are different from a portion of or the entire mouse IL10 nucleotide sequence (e.g., exon 1, exon 2, exon 3, exon 4, exon 5, or SEQ ID NO: 1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, or 60 nucleotides, e.g., contiguous or non-contiguous nucleotides) that is the same as a portion of or the entire mouse IL10 nucleotide sequence (e.g., exon 1, exon 2, exon 3, exon 4, exon 5, or SEQ ID NO: 1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire human IL10 nucleotide sequence (e.g., exon 1, exon 2, exon 3, exon 4, exon 5, or SEQ ID NO: 3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL10 nucleotide sequence (e.g., exon 1, exon 2, exon 3, exon 4, exon 5, or SEQ ID NO: 3).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL10 amino acid sequence (e.g., an amino acid sequence encoded by exon 1, exon 2, exon 3, exon 4, exon 5; or SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL10 amino acid sequence (e.g., an amino acid sequence encoded by exon 1, exon 2, exon 3, exon 4, exon 5; or SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL10 amino acid sequence (e.g., an amino acid sequence encoded by exon 1, exon 2, exon 3, exon 4, exon 5; or SEQ ID NO: 4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL10 amino acid sequence (e.g., an amino acid sequence encoded by exon 1, exon 2, exon 3, exon 4, exon 5; or SEQ ID NO: 4).

In some embodiments, the percentage identity of any of the amino acid sequence described herein with the sequence shown in SEQ ID NO: 2 or 4 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

Cells, tissues, and animals (e.g., mouse) are also provided that comprise the nucleotide sequences as described herein, as well as cells, tissues, and animals (e.g., mouse) that express human or chimeric (e.g., humanized) IL10 from an endogenous non-human IL10 locus.

In one aspect, the disclosure provides a genetically-modified, non-human animal whose genome comprises at least one chromosome comprising a sequence encoding a human or chimeric IL10.

In some embodiments, the sequence encoding the human or chimeric IL10 is operably linked to an endogenous regulatory element, or a human regulatory element at the endogenous IL10 gene locus in the at least one chromosome.

In some embodiments, the sequence encoding a human or chimeric IL10 comprises a sequence encoding an amino acid sequence that is at least 50%, 55%, 65%, 70%, 75%, 80%, 85%, 90%, 95%, 99%, or 100% identical to human IL10 (SEQ ID NO: 4).

In some embodiments, the animal is a mammal, e.g., a monkey, a rodent or a mouse. In some embodiments, the animal is a BALB/c mouse or a C57BL/6 mouse.

In some embodiments, the animal does not express endogenous IL10. In some embodiments, the animal has one or more cells expressing human or chimeric IL10. In some embodiments, the animal has at least 50%, at least 60%, at least 70%, at least 80%, at least 90% or at least 95% cells (e.g., monocytes, lymphocytes, type-II T helper cells (TH2), mast cells, CD4+CD25+Foxp3+ regulatory T cells, activated T cells and/or activated B cells) expressing human or chimeric IL10.

In some embodiments, the animal has one or more cells expressing human or chimeric IL10, and the expressed human or chimeric IL10 can bind to endogenous IL10R complex or IL10RA. In some embodiments, the animal has one or more cells expressing human or chimeric IL10, and the expressed human or chimeric IL10 cannot bind to endogenous IL10R complex or IL10RA.

In another aspect, the disclosure is related to a genetically-modified, non-human animal, wherein the genome of the animal comprises a replacement of a sequence encoding a region of endogenous IL10 with a sequence encoding a corresponding region of human IL10 at an endogenous IL10 gene locus.

In some embodiments, the sequence encoding the corresponding region of human IL10 is operably linked to an endogenous regulatory element, or a human regulatory element at the endogenous IL10 locus, and one or more cells of the animal expresses a chimeric IL10.

In some embodiments, the animal is a mouse, and the replaced endogenous IL10 locus is exon 1, exon 2, exon 3, exon 4, and/or exon 5 of the endogenous mouse IL10 gene.

In some embodiments, the animal is heterozygous with respect to the replacement at the endogenous IL10 gene locus. In some embodiments, the animal is homozygous with respect to the replacement at the endogenous IL10 gene locus.

In another aspect, the disclosure is related to methods for making a genetically-modified, non-human animal. The methods involve replacing in at least one cell of the animal, at an endogenous IL10 gene locus, a sequence encoding a region of an endogenous IL10 with a sequence encoding a corresponding region of human IL10.

In some embodiments, the sequence encoding the corresponding region of human IL10 comprises exon 1, exon 2, exon 3, exon 4, and/or exon 5 of a human IL10 gene.

In some embodiments, the sequence encoding the corresponding region of IL10 comprises at least 50, 75, 100, 125, 150, 175, or 200 nucleotides of exon 1, exon 2, exon 3, exon 4, and/or exon 5 of a human IL10 gene.

In some embodiments, the sequence encoding the corresponding region of human IL10 encodes a sequence that is at least 90% identical to full-length amino acid sequence of SEQ ID NO: 4.

In some embodiments, the animal is a mouse, and the locus is exon 1, exon 2, exon 3, exon 4, and/or exon 5 of the mouse IL10 gene.

In another aspect, the disclosure is also related to a non-human animal comprising at least one cell comprising a nucleotide sequence encoding a chimeric IL10 polypeptide, wherein the chimeric IL10 polypeptide comprises at least 50, 60, 70, 80, 90, 100, 110, 120, 130, 140, 150, 160, or 170 contiguous amino acid residues that are identical to the corresponding contiguous amino acid sequence of a human IL10, wherein the animal expresses the chimeric IL10.

In some embodiments, the chimeric IL10 polypeptide comprises a sequence that is at least 90%, 95%, or 99% identical to full-length amino acid sequence of SEQ ID NO: 4.

In some embodiments, the nucleotide sequence is operably linked to an endogenous IL10 regulatory element of the animal, a human IL10 regulatory element, an endogenous 5′-UTR, an endogenous 3′-UTR, a human 5′-UTR, or a human 3′-UTR.

In some embodiments, the nucleotide sequence is integrated to an endogenous IL10 gene locus of the animal.

In some embodiments, the chimeric IL10 has at least one mouse IL10 activity and/or at least one human IL10 activity.

In another aspect, the disclosure is also related to methods of making a genetically-modified mouse cell that expresses a chimeric IL10. The methods involve replacing, at an endogenous mouse IL10 gene locus, a nucleotide sequence encoding a region of mouse IL10 with a nucleotide sequence encoding a corresponding region of human IL10, thereby generating a genetically-modified mouse cell that includes a nucleotide sequence that encodes the chimeric IL10, wherein the mouse cell expresses the chimeric IL10.

In some embodiments, the nucleotide sequence encoding the chimeric IL10 is operably linked to an endogenous regulatory region, or a human IL10 regulatory region, e.g., promoter.

In some embodiments, the animal further comprises a sequence encoding an additional human or chimeric protein (e.g., IL10R, IL10RA, IL10Rb, Interleukin 33 (IL33), Interleukin 3 (IL3), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal Regulatory Protein alpha (SIRPa)).

In some embodiments, the additional human or chimeric protein is IL10R (e.g., IL10RA).

In one aspect, the disclosure also provides methods of determining effectiveness of an IL10 pathway agonist (e.g., an anti-IL10 antibody) for reducing inflammation. The methods involve administering the IL10 pathway agonist to the animal described herein, wherein the animal has an inflammation; and determining the inhibitory effects of the IL10 pathway agonist to the reduction of inflammation.

In one aspect, the disclosure also provides methods of determining effectiveness of an IL10 pathway agonist (e.g., an anti-IL10 antibody) for treating autoimmune disorder or allergy. The methods involve administering the IL10 pathway agonist to the animal described herein, wherein the animal has an autoimmune disorder or allergy; and determining the inhibitory effects of the IL10 pathway agonist to the treatment of autoimmune disorder or allergy.

In one aspect, the disclosure also provides methods of determining effectiveness of an IL10 pathway antagonist (e.g., an anti-IL10 antibody) for treating cancer. The methods involve administering the IL10 pathway antagonist to the animal as described herein, wherein the animal has a tumor; and determining the inhibitory effects of the IL10 pathway antagonist to the tumor.

In some embodiments, the animal further comprises a sequence encoding a human or chimeric IL10R (e.g., IL10RA). In some embodiments, the additional therapeutic agent is an anti-IL10R antibody (anti-IL10RA antibody).

In some embodiments the additional therapeutic agent is an anti-PD-1 antibody, an anti-PD-L1 antibody, an anti-CTLA4 antibody, an anti-CD20 antibody, an anti-EGFR antibody, or an anti-CD319 antibody.

In another aspect, the disclosure further provides methods of determining toxicity of an agent (e.g., an IL10 pathway antagonist). The methods involve administering the agent to the animal as described herein; and determining weight change of the animal. In some embodiments, the method further involve performing a blood test (e.g., determining red blood cell count).

In one aspect, the disclosure relates to proteins comprising an amino acid sequence, wherein the amino acid sequence is one of the following:

a) an amino acid sequence shown in SEQ ID NO: 2 or 4;

b) an amino acid sequence having a homology of at least 90% with or at least 90% identical to the amino acid sequence shown in SEQ ID NO: 2 or 4;

c) an amino acid sequence encoded by a nucleic acid sequence, wherein the nucleic acid sequence is able to hybridize to a nucleotide sequence encoding the amino acid shown in SEQ ID NO: 2 or 4 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2 or 4;

e) an amino acid sequence that is different from the amino acid sequence shown in SEQ ID NO: 2 or 4 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 2 or 4.

In some embodiments, provided herein are cells comprising the proteins disclosed herein. In some embodiments, provided herein are animals having the proteins disclosed herein.

In another aspect, the disclosure relates to nucleic acids comprising a nucleotide sequence, wherein the nucleotide sequence is one of the following:

a) a nucleic acid sequence as shown in SEQ ID NO: 7, or a nucleic acid sequence encoding a homologous IL10 amino acid sequence of a humanized mouse IL10;

b) a nucleic acid sequence that is able to hybridize to the nucleotide sequence as shown in SEQ ID NO: 1, 3, 5, 6, 7, 8, 9, or 52 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 1, 3, 5, 6, 7, 8, 9, or 52;

d) a nucleic acid sequence that encodes an amino acid sequence, wherein the amino acid sequence has a homology of at least 90% with or at least 90% identical to the amino acid sequence shown in SEQ ID NO: 1, 3, or 7;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 3, or 7;

f) a nucleic acid sequence that encodes an amino acid sequence, wherein the amino acid sequence is different from the amino acid sequence shown in SEQ ID NO: 2 or 4 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 2 or 4.

The present disclosure further relates to a DNA sequence of a humanized mouse. The DNA sequence is obtained by reverse transcription of the mRNA obtained by transcription thereof is consistent with or complementary to the DNA sequence homologous to the sequence shown in SEQ ID NO: 1, 3, or 7.

The disclosure also provides an amino acid sequence that has a homology of at least 90% with, or at least 90% identical to the sequence shown in SEQ ID NO: 2 or 4, and has protein activity. In some embodiments, the homology with the sequence shown in SEQ ID NO: 2 or 4 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 1, 3, or 7, and encodes a polypeptide that has protein activity. In some embodiments, the homology with the sequence shown in SEQ ID NO: 1, 3, or 7 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous IL10 gene, wherein the disruption of the endogenous IL10 gene comprises deletion of exon 1, exon2, exon 3, exon 4, and/or exon 5 or part thereof of the endogenous IL10 gene.

In some embodiments, the disruption of the endogenous IL10 gene further comprises deletion of one or more exons or part of exons selected from the group consisting of exon 1, exon 2, exon 3, exon 4, and/or exon 5 of the endogenous IL10 gene.

In some embodiments, the disruption of the endogenous IL10 gene further comprises deletion of one or more introns or part of introns selected from the group consisting of intron 1, intron 2, intron 3, and/or intron 4 of the endogenous IL10 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500 or more nucleotides.

In some embodiments, the disruption of the endogenous IL10 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleotides of exon 1, exon 2, exon 3, exon 4, and/or exon 5 (e.g., deletion of the entire exon 1, exon 2, exon 3, exon 4, and exon 5).

### Interleukin 10 Receptor (IL10R)

Interleukin-10 receptor (IL10R, or IL-10R) is a type II cytokine receptor. The receptor is tetrameric, composed of 2 alpha and 2 beta subunits. The alpha subunit is expressed on haematopoietic cells (such as T, B, NK, mast, and dendritic cells) whilst the β subunit is expressed ubiquitously. IL10RA (IL-10 receptor alpha subunit; or IL10RA) is 90-120 kDa and serves as the ligand binding subunit of the receptor complex. IL10Rb (IL-10 receptor beta subunit; or IL10RB) is the signaling subunit of the IL10R complex and is constitutively expressed in most cell types. Earlier studies have suggested that IL10Rb has almost no role in IL10-binding; its main role is to recruit the downstream signaling kinases. Studies have found that upon binding to IL10, IL10RA induces a conformational change in IL10Rb, permitting IL10Rb to also bind IL10. Unlike IL10RA, which is unique to IL10, the IL10Rb-subunit is shared by receptors for other type-II cytokines including IL22, IL26, and INF-λ.

IL-10 cellular responses require the specific recognition and assembly of a cell surface complex comprised of IL10RA and IL-10Rb chains. IL-10Ra is an ˜80,000 kDa protein with an extracellular ligand binding domain (ECD) of 227 residues, a transmembrane helix of 21 residues, and an intracellular domain (ICD) of 322 amino acids. The ECD of IL10Rb is about the same length as IL10RA, consisting of 201 residues. However, the ICD of IL-10Rb consists of only 83 residues. The IL-10Ra ECD forms specific high affinity interactions (KD=50-200 pM) with IL-10, while IL-10Rb is a low affinity (˜mM) shared receptor that participates in receptor complexes with other class 2 cytokine family members.

The sequence of receptor assembly is initiated by IL10 binding to IL10RA. This complex then binds IL10Rb forming a heterotetramer, permitting the assembly of the signaling complex. Once the complex is assembled, tyrosine kinases Jak1 and Tyk2 that are constitutively associated with IL10RA and IL10Rb, respectively, are activated and phosphorylate specific tyrosine residues in the intracellular domain of IL10RA. Phosphorylation of the receptor leads to the recruitment of signal transducer and activator of transcription 3 (STAT3). Following their recruitment, JAK1 and TYK2 phosphorylate STAT3, leading to its homodimerization and subsequent translocation to the nucleus, where it binds to STAT3-binding elements of IL10-responsive genes.

A detailed description of IL10R and its function can be found, e.g., in Shouval, D. S. et al., “Interleukin 10 receptor signaling: master regulator of intestinal mucosal homeostasis in mice and humans.” Advances in Immunology. Vol. 122. Academic Press, 2014. 177-210; Walter, M. R., “The molecular basis of IL-10 function: from receptor structure to the onset of signaling.” Interleukin-10 in Health and Disease. Springer, Berlin, Heidelberg, 2014. 191-212; each of which is incorporated by reference herein in the entirety.

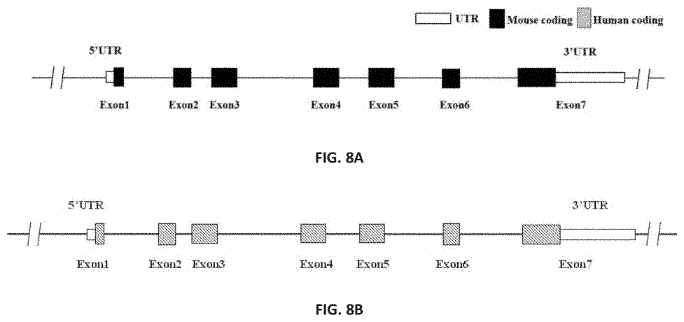

In human genomes, IL10RA gene (Gene ID: 3587) locus has 7 exons, exon 1, exon 2, exon 3, exon 4, exon 5, exon 6, and exon 7 (FIG. 8B). The IL10RA protein has an extracellular region, a transmembrane region, and a cytoplasmic region. The nucleotide sequence for human IL10RA mRNA is NM_001558.3 (SEQ ID NO: 27), and the amino acid sequence for human IL10RA is NP_001549.2 (SEQ ID NO: 28). The location for each exon and each region in human IL10RA nucleotide sequence and amino acid sequence is listed below:

TABLE 3

| | NM_001558.3 | NP_001549.2 |
|---|---|---|
| Human IL10RA | 3672 bp | 578 aa |
| (approximate location) | (SEQ ID NO: 27) | (SEQ ID NO: 28) |
| Exon 1 | 1-144 | 1-22 |
| Exon 2 | 145-265 | 23-63 |
| Exon 3 | 266-444 | 64-122 |
| Exon 4 | 445-614 | 123-179 |
| Exon 5 | 615-765 | 180-229 |
| Exon 6 | 766-887 | 230-270 |
| Exon 7 | 888-3656 | 271-578 |
| Signal peptide | 78-140 | 1-21 |
| Extracellular | 141-782 | 22-235 |
| Transmembrane region | 783-845 | 236-256 |
| Cytoplasmic | 846-1811 | 257-578 |
| Donor region in Example | 78-869 | 1-264 |

In mice, IL10RA gene locus has 7 exons, exon 1, exon 2, exon 3, exon 4, exon 5, exon 6, and exon 7 (FIG. 8A). The mouse IL10RA protein also has an extracellular region, a transmembrane region, and a cytoplasmic region. The nucleotide sequence for mouse IL10RA mRNA is NM_008348.3 (SEQ ID NO: 25), the amino acid sequence for mouse IL10RA is NP_032374.1 (SEQ ID NO: 26). The location for each exon and each region in the mouse IL10RA nucleotide sequence and amino acid sequence is listed below:

TABLE 4

| | NM_008348.3 | NP_032374.1 |
|---|---|---|
| Mouse IL10RA | 3509 bp | 575 aa |
| (approximate location) | (SEQ ID NO: 25) | (SEQ ID NO: 26) |
| Exon 1 | 1-144 | 1-22 |
| Exon 2 | 145-265 | 23-63 |
| Exon 3 | 266-453 | 64-125 |
| Exon 4 | 454-620 | 126-181 |
| Exon 5 | 621-774 | 182-232 |
| Exon 6 | 775-893 | 233-272 |
| Exon 7 | 894-3498 | 273-575 |
| Signal peptide | 78-125 | 1-16 |
| Extracellular | 126-800 | 17-241 |
| Transmembrane region | 801-863 | 242-262 |
| Cytoplasmic | 864-1802 | 263-575 |
| Replaced region in Example | 78-875 | 1-266 |

The mouse IL10RA gene (Gene ID: 16154) is located in Chromosome 9 of the mouse genome, which is located from 45,253,837 to 45,269,149 of NC_000075.6 (GRCm38: CM00099 4.2). The 5′-UTR is from 45,269,149 to 45,269,073, exon 1 is from 45,269,149 to 45,269,006, the first intron is from 45,269,005 to 45,267,221, exon 2 is from 45,267,220 to 45,267,100, the second intron is from 45,267,099 to 45,266,643, exon 3 is from 45,266,642 to 45,266,455, the third intron is from 45,266,454 to 45,265,656, exon 4 is from 45,265,655 to 45,265,489, the fourth intron is from 45,265,488 to 45,264,479, exon 5 is from 45,264,478 to 45,264,325, the fifth intron is from 45,264,324 to 45,260,472, exon 6 is from 45,260,471 to 45,260,353, the sixth intron is from 45,260,352 to 45,256,442, exon 7 is from 45,256,441 to 45,253,837, the 3′-UTR is from 45,255,529 to 45,253,837, based on transcript NM_008348.3. All relevant information for mouse IL10RA locus can be found in the NCBI website with Gene ID: 16154, which is incorporated by reference herein in its entirety.

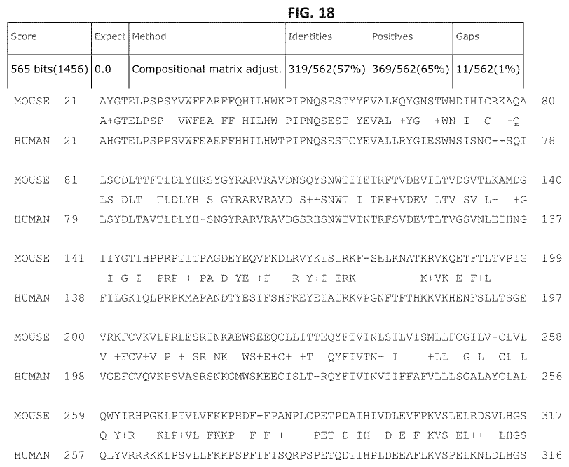

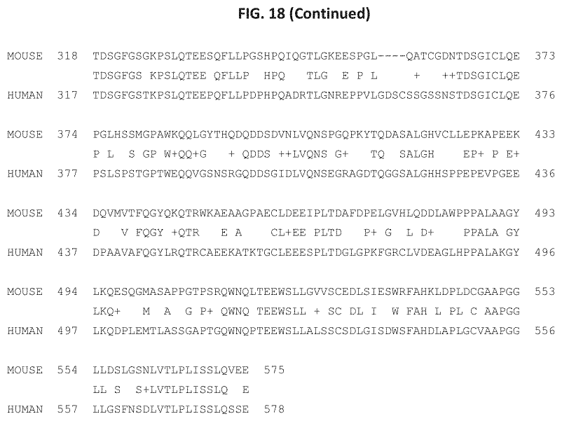

FIG. 18 shows the alignment between mouse IL10RA amino acid sequence (NP_032374.1; SEQ ID NO: 26) and human IL10RA amino acid sequence (NP_001549.2; SEQ ID NO: 28). Thus, the corresponding amino acid residue or region between mouse and human IL10RA can also be found in FIG. 18.

IL10RA genes, proteins, and locus of the other species are also known in the art. For example, the gene ID for IL10RA in Rattus norvegicus is 117539, the gene ID for IL10RA in Macaca mulatta (Rhesus monkey) is 704383, the gene ID for IL10RA in Canis lupus familiaris (dog) is 610823, and the gene ID for IL10RA in Sus scrofa (pig) is 100525654. The relevant information for these genes (e.g., intron sequences, exon sequences, amino acid residues of these proteins) can be found, e.g., in NCBI database.

The present disclosure provides human or chimeric (e.g., humanized) IL10RA nucleotide sequence and/or amino acid sequences. In some embodiments, the entire sequence of mouse exon 1, exon 2, exon 3, exon 4, exon 5, exon 6, exon 7, the signal peptide, the extracellular region, the transmembrane region, and/or the cytoplasmic region are replaced by the corresponding human sequence.

In some embodiments, the human or chimeric (e.g., humanized) IL10RA forms a functional IL10R complex with the endogenous IL10Rb.

In some embodiments, a “region” or “portion” of mouse exon 1, exon 2, exon 3, exon 4, exon 5, exon 6, exon 7, signal peptide, the extracellular region, the transmembrane region, and/or the cytoplasmic region is replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 150, 200, 250, 300, 350, or 400 nucleotides, or at least 1, 2, 3, 4, 5, 6, 7, 8, 9, 10, 20, 30, 40, 50, 60, 70, 80, 90, 100, 110, 120, 130, or 150 amino acid residues.

In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, signal peptide, the extracellular region, the transmembrane region, and/or the cytoplasmic region. In some embodiments, a region, a portion, or the entire sequence of mouse exon 1, exon 2, exon 3, exon 4, exon 5, exon 6, and/or exon 7 (e.g., a part of exon 1, exon 2, exon 3, exon 4, exon 5, and a part of exon 6) is replaced by a region, a portion, or the entire sequence of human exon 1, exon 2, exon 3, exon 4, exon 5, exon 6, and/or exon 7 (e.g., a part of exon 1, exon 2, exon 3, exon 4, exon 5, and a part of exon 6).

In some embodiments, a “region” or “portion” of mouse exon 1, exon 2, exon 3, exon 4, exon 5, exon 6, exon 7, signal peptide, the extracellular region, the transmembrane region, and/or the cytoplasmic region is deleted. For example, a region or a portion of exon 1, exon 2, exon 3, exon 4, exon 5, and exon 6 is deleted.

Thus, in some embodiments, the present disclosure also provides a chimeric (e.g., humanized) IL10RA nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL10RA mRNA sequence (e.g., SEQ ID NO: 25), mouse IL10RA amino acid sequence (e.g., SEQ ID NO: 26), or a portion thereof (e.g., exon 1, exon 2, exon 3, exon 4, exon 5, exon 6, and/or exon 7). In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL10RA mRNA sequence (e.g., SEQ ID NO: 27), human IL10RA amino acid sequence (e.g., SEQ ID NO: 28), or a portion thereof (e.g., exon 1, exon 2, exon 3, exon 4, exon 5, exon 6, and/or exon 7).

In some embodiments, the sequence encoding amino acids 1-266 of mouse IL10RA (SEQ ID NO: 26) is replaced. In some embodiments, the sequence is replaced by a sequence encoding a corresponding region of human IL10RA (e.g., amino acids 1-264 of human IL10RA (SEQ ID NO: 28).

In some embodiments, the sequence encoding amino acids 17-266 of mouse IL10RA (SEQ ID NO: 26) is replaced. In some embodiments, the sequence is replaced by a sequence encoding a corresponding region of human IL10RA (e.g., amino acids 22-264 of human IL10RA (SEQ ID NO: 28).

In some embodiments, the sequence encoding amino acids 17-241 of mouse IL10RA (SEQ ID NO: 26) is replaced. In some embodiments, the sequence is replaced by a sequence encoding a corresponding region of human IL10RA (e.g., amino acids 22-235 of human IL10RA (SEQ ID NO: 28).

In some embodiments, the nucleic acids as described herein are operably linked to a promotor or regulatory element, e.g., an endogenous mouse IL10RA promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse IL10RA nucleotide sequence (e.g., exon 1, exon 2, exon 3, exon 4, exon 5, exon 6, exon 7, or SEQ ID NO: 25).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL10RA nucleotide sequence (e.g., exon 1, exon 2, exon 3, exon 4, exon 5, exon 6, exon 7, or SEQ ID NO: 25).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire human IL10RA nucleotide sequence (e.g., exon 1, exon 2, exon 3, exon 4, exon 5, exon 6, exon 7, or SEQ ID NO: 27).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL10RA nucleotide sequence (e.g., exon 1, exon 2, exon 3, exon 4, exon 5, exon 6, exon 7, or SEQ ID NO: 27).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL10RA amino acid sequence (e.g., an amino acid sequence encoded by exon 1, exon 2, exon 3, exon 4, exon 5, exon 6, exon 7; or NP_032374.1 (SEQ ID NO: 26)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL10RA amino acid sequence (e.g., an amino acid sequence encoded by exon 1, exon 2, exon 3, exon 4, exon 5, exon 6, exon 7; or NP_032374.1 (SEQ ID NO: 26)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL10RA amino acid sequence (e.g., an amino acid sequence encoded by exon 1, exon 2, exon 3, exon 4, exon 5, exon 6, exon 7; or NP_001549.2 (SEQ ID NO: 28)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL10RA amino acid sequence (e.g., exon 1, exon 2, exon 3, exon 4, exon 5, exon 6, exon 7, or NP_001549.2 (SEQ ID NO: 28)).

The present disclosure also provides a humanized IL10RA mouse amino acid sequence, wherein the amino acid sequence is selected from the group consisting of:

a) an amino acid sequence shown in SEQ ID NO: 28 or 32;

b) an amino acid sequence having a homology of at least 90% with or at least 90% identical to the amino acid sequence shown in SEQ ID NO: 28 or 32;

c) an amino acid sequence encoded by a nucleic acid sequence, wherein the nucleic acid sequence is able to hybridize to a nucleotide sequence encoding the amino acid shown in SEQ ID NO: 28 or 32 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 28 or 32;

e) an amino acid sequence that is different from the amino acid sequence shown in SEQ ID NO: 28 or 32 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 28 or 32.

The present disclosure also relates to a nucleic acid (e.g., DNA or RNA) sequence, wherein the nucleic acid sequence can be selected from the group consisting of:

a) a nucleic acid sequence as shown in SEQ ID NO: 31, or a nucleic acid sequence encoding a homologous IL10RA amino acid sequence of a humanized mouse;

b) a nucleic acid sequence that is able to hybridize to the nucleotide sequence as shown in SEQ ID NO: 25, 27, 29, 30, 31, 33, 34, or 53 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 25, 27, 29, 30, 31, 33, 34, or 53;

d) a nucleic acid sequence that encodes an amino acid sequence, wherein the amino acid sequence has a homology of at least 90% with or at least 90% identical to the amino acid sequence shown in SEQ ID NO: 25, 27, or 31;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 28 or 32;

f) a nucleic acid sequence that encodes an amino acid sequence, wherein the amino acid sequence is different from the amino acid sequence shown in SEQ ID NO: 28 or 32 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 28 or 32.

The present disclosure further relates to an IL10RA genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 25, 27, or 31.

The disclosure also provides an amino acid sequence that has a homology of at least 90% with, or at least 90% identical to the sequence shown in SEQ ID NO: 26, 28 or 32, and has protein activity. In some embodiments, the homology with the sequence shown in SEQ ID NO: 26, 28 or 32 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 25, 27, or 31, and encodes a polypeptide that has protein activity. In some embodiments, the homology with the sequence shown in SEQ ID NO: 25, 27, or 31 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

Cells, tissues, and animals (e.g., mouse) are also provided that comprise the nucleotide sequences as described herein, as well as cells, tissues, and animals (e.g., mouse) that express human or chimeric (e.g., humanized) IL10RA from an endogenous non-human IL10RA locus.

In one aspect, the disclosure also provides methods of determining effectiveness of an IL10R agonist (e.g., an anti-IL10R antibody or an anti-IL10RA antibody) for reducing inflammation. The methods involve administering the IL10R agonist to the animal described herein, wherein the animal has an inflammation; and determining the inhibitory effects of the IL10R agonist to the reduction of inflammation.

In one aspect, the disclosure also provides methods of determining effectiveness of an IL10R agonist (e.g., an anti-IL10R antibody) for treating autoimmune disorder or allergy. The methods involve administering the IL10R agonist to the animal described herein, wherein the animal has an autoimmune disorder or allergy; and determining the inhibitory effects of the IL10R agonist to the treatment of autoimmune disorder or allergy.

In one aspect, the disclosure also provides methods of determining effectiveness of an IL10R antagonist (e.g., an anti-IL10R antibody or an anti-IL10RA antibody) for treating cancer. The methods involve administering the IL10R antagonist to the animal described herein, wherein the animal has a tumor; and determining the inhibitory effects of the IL10R antagonist to the tumor. In some embodiments, the tumor comprises one or more cancer cells that are injected into the animal. In some embodiments, determining the inhibitory effects of the IL10R antagonist (e.g., an anti-IL10R antibody) to the tumor involves measuring the tumor volume in the animal.

In another aspect, the disclosure also provides a genetically-modified, non-human animal whose genome comprise a disruption in the animal's endogenous IL10R gene (e.g., IL10RA or IL10Rb). In some embodiments, the disruption of the endogenous IL10R gene comprises deletion of exon 1, exon 2, exon 3, exon 4, exon 5, exon 6, exon 7 (e.g., exon 1, exon 2, exon 3, exon 4, exon 5, and/or exon 6) or part thereof of the endogenous IL10R gene (e.g., IL10RA).

In some embodiments, the disruption of the endogenous IL10R gene comprises deletion of one or more exons or part of exons selected from the group consisting of exon 1, exon 2, exon 3, exon 4, exon 5, and/or exon 6 of the endogenous IL10R gene (e.g., IL10RA).

In some embodiments, the disruption of the endogenous IL10R gene further comprises deletion of one or more introns or part of introns selected from the group consisting of intron 1, intron 2, intron 3, intron 4, intron 5, and/or intron 6 of the endogenous IL10R gene (e.g., IL10RA).

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500 or more nucleotides.

In some embodiments, the disruption of the endogenous IL10R gene (e.g., IL10RA) comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleotides of exon 1, exon 2, exon 3, exon 4, exon 5, exon 6, exon 7 (e.g., exon 1, exon 2, exon 3, exon 4, exon 5, and/or exon 6).

### Genetically Modified Animals

As used herein, the term “genetically-modified non-human animal” refers to a non-human animal having genetic modification (e.g., exogenous DNA) in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the genetic modification in its genome. The cell having exogenous DNA can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a modified endogenous IL10R (e.g., IL10RA) locus or IL10 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one portion of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized IL10R gene (e.g., IL10RA) or a humanized IL10R (e.g., IL10RA) nucleic acid. In some embodiments, at least one or more portions of the gene or the nucleic acid is from the human IL10R gene (e.g., IL10RA), at least one or more portions of the gene or the nucleic acid is from a non-human IL10R gene (e.g., IL10RA). In some embodiments, the gene or the nucleic acid comprises a sequence that encodes an IL10R protein (e.g., IL10RA). In some embodiments, the encoded IL10RA protein is functional or has at least one activity of the human IL10RA protein or the non-human IL10RA protein, e.g., binding to human or non-human IL10, and/or downregulating immune response.

In some embodiments, the chimeric protein or the chimeric polypeptide is a humanized IL10RA protein or a humanized IL10RA polypeptide. In some embodiments, at least one or more portions of the amino acid sequence of the protein or the polypeptide is from a human IL10RA protein, and at least one or more portions of the amino acid sequence of the protein or the polypeptide is from a non-human IL10RA protein. The humanized IL10RA protein or the humanized IL10RA polypeptide is functional or has at least one activity of the human IL10RA protein or the non-human IL10RA protein.

In some embodiments, the humanized IL10RA protein or the humanized IL10RA polypeptide can bind to mouse IL10, and/or downregulating immune response. In some embodiments, the humanized IL10RA protein or the humanized IL10RA polypeptide cannot bind to mouse IL10, thus cannot downregulate immune response.

In some embodiments, the chimeric gene or the chimeric nucleic acid is a humanized IL10 gene or a humanized IL10 nucleic acid. In some embodiments, at least one or more portions of the gene or the nucleic acid is from the human IL10 gene, at least one or more portions of the gene or the nucleic acid is from a non-human IL10 gene. In some embodiments, the gene or the nucleic acid comprises a sequence that encodes an IL10 protein. The encoded IL10 protein is functional or has at least one activity of the human IL10 protein or the non-human IL10 protein, e.g., binding to human or non-human IL10R complex, and/or downregulating immune response.

In some embodiments, the chimeric protein or the chimeric polypeptide is a humanized IL10 protein or a humanized IL10 polypeptide. In some embodiments, at least one or more portions of the amino acid sequence of the protein or the polypeptide is from a human IL10 protein, and at least one or more portions of the amino acid sequence of the protein or the polypeptide is from a non-human IL10 protein. The humanized IL10 protein or the humanized IL10 polypeptide is functional or has at least one activity of the human IL10 protein or the non-human IL10 protein.

In some embodiments, the humanized IL10 protein or the humanized IL10 polypeptide can bind to mouse IL10R complex, and/or downregulate immune response. In some embodiments, the humanized IL10 protein or the humanized IL10 polypeptide cannot bind to mouse IL10R complex, thus cannot downregulate immune response.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL10R (e.g., IL10RA) or IL10 animal is made. For example, suitable mice for maintaining a xenograft, can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Ry knockout mice, NOD/SCID/γc^{null} mice (Ito, M. et al., NOD/SCID/γc^{null} mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL10R (e.g., IL10RA) or IL10 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Ry knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature IL10R (e.g., IL10RA) or IL10 coding sequence with human mature IL10R (e.g., IL10RA) or IL10 coding sequence.

Genetically modified non-human animals can comprise a modification of an endogenous non-human IL10 or IL10R (e.g., IL10RA) locus. In some embodiments, the modification can comprise a human nucleic acid sequence encoding at least a portion of a mature IL10 or IL10R (e.g., IL10RA) protein (e.g., at least 10%, 20%, 30%, 40%, 50%, 60%, 70%, 80%, 90%, 95%, 96%, 97%, 98%, or 99% identical to the mature IL10 or IL10R (e.g., IL10RA) protein sequence). Although genetically modified cells are also provided that can comprise the modifications described herein (e.g., ES cells, somatic cells), in many embodiments, the genetically modified non-human animals comprise the modification of the endogenous IL10 or IL10R (e.g., IL10RA) locus in the germline of the animal.

Genetically modified animals can express a human IL10 or IL10R (e.g., IL10RA) (or a chimeric IL10 or IL10R (e.g., IL10RA)) from endogenous mouse loci, wherein the endogenous mouse gene has been replaced with a human gene and/or a nucleotide sequence that encodes a region of human IL10 or IL10R (e.g., IL10RA) sequence or an amino acid sequence that is at least 10%, 20%, 30%, 40%, 50%, 60%, 70&, 80%, 90%, 95%, 96%, 97%, 98%, or 99% identical to the human IL10 or IL10R (e.g., IL10RA) sequence. In various embodiments, an endogenous non-human locus is modified in whole or in part to comprise human nucleic acid sequence encoding at least one protein-coding sequence of a mature protein.

In some embodiments, the genetically modified mice express the human IL10 or IL10R (e.g., IL10RA) (or chimeric IL10 or IL10R (e.g., IL10RA)) from endogenous loci that are under control of mouse promoters and/or mouse regulatory elements. The replacement(s) at the endogenous mouse loci provide non-human animals that express human protein or chimeric protein in appropriate cell types and in a manner that does not result in the potential pathologies observed in some other transgenic mice known in the art. The human protein or the chimeric protein expressed in animal can maintain one or more functions of the wild-type mouse or human protein in the animal. For example, IL10R can bind to human or non-human IL10, and downregulate immune response, e.g., downregulate immune response by at least 10%, 20%, 30%, 40%, or 50%. As used herein, the term “endogenous IL10R” refers to IL10R protein (e.g., IL10RA) that is expressed from an endogenous IL10R nucleotide sequence of the non-human animal (e.g., mouse) before any genetic modification. Similarly, the term “endogenous IL10” refers to IL10 protein that is expressed from an endogenous IL10 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to SEQ ID NO: 40, 42, or 44, and/or a sequence encoding an amino acid sequence that is at least 70%, 75%, 80%, 85%, 90%, 95%, 99%, or 100% identical to SEQ ID NO: 4.

The genome of the genetically modified animal can comprise a replacement at an endogenous IL10R (e.g., IL10RA) gene locus of a sequence encoding a region of endogenous IL10R (e.g., IL10RA) with a sequence encoding a corresponding region of human IL10R (e.g., IL10RA). In some embodiments, the sequence that is replaced is any sequence within the endogenous IL10R (e.g., IL10RA) gene locus, e.g., exon 1, exon 2, exon 3, exon 4, exon 5, exon 6, exon 7, 5′-UTR, 3′UTR, the first intron, the second intron, and the third intron, the fourth intron, the fifth intron, the sixth intron, etc. In some embodiments, the sequence that is replaced is within the regulatory region of the endogenous IL10R gene (e.g., IL10RA). In some embodiments, the sequence that is replaced is exon 1, exon 2, exon 3, exon 4, exon 5, and/or exon 6, or part thereof, of an endogenous mouse IL10R gene (e.g., IL10RA) locus.

The genetically modified animal can have one or more cells expressing a human or chimeric IL10R (e.g., humanized IL10R) having an extracellular region, a transmembrane region, and a cytoplasmic region. In some embodiments, the extracellular region of the human or chimeric IL10R (e.g., IL10RA) described herein comprises a sequence that is at least or about 50%, 60%, 70%, 80%, 90%, 95%, 99% identical to the extracellular region of human IL10R (e.g., IL10RA). In some embodiments, the extracellular region of the human or chimeric IL10R (e.g., IL10RA) has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to human IL10R (e.g., IL10RA). In some embodiments, the extracellular region of the human or chimeric IL10R (e.g., IL10RA) comprises a sequence that is the entire or a part of amino acids 22-235 of SEQ ID NO: 28.

In some embodiments, the transmembrane region of the human or chimeric IL10R (e.g., IL10RA) described herein comprises a sequence that is at least or about 50%, 60%, 70%, 80%, 90%, 95%, 99% identical to the transmembrane region of human IL10R (e.g., IL10RA). In some embodiments, the transmembrane region of the human or chimeric IL10R (e.g., IL10RA) has a sequence that has at least 1, 2, 3, 4, 5, 6, 7, 8, 9, 10, 15 or 20 amino acids (e.g., contiguously or non-contiguously) that are identical to human IL10R (e.g., IL10RA). In some embodiments, the transmembrane region of the human or chimeric IL10R (e.g., IL10RA) comprises a sequence that is the entire of a part of amino acids 236-256 of SEQ ID NO: 28.

In some embodiments, the cytoplasmic region of the human or chimeric IL10R (e.g., IL10RA) described herein comprises a sequence that is at least or about 50%, 60%, 70%, 80%, 90%, 95%, 99% identical to the cytoplasmic region of human IL10R (e.g., IL10RA). In some embodiments, the cytoplasmic region of the human or chimeric IL10R (e.g., IL10RA) has a sequence that has at least 10, 20, 30, 40, 50, 60, 70, 80, 90, 100, 110, 120, 130, 140, 150, 160, 170, 180, 190, 200, 250, or 300 amino acids (e.g., contiguously or non-contiguously) that are identical to human IL10R (e.g., IL10RA). In some embodiments, the cytoplasmic region of the human or chimeric IL10R (e.g., IL10RA) comprises a sequence that is the entire of a part of amino acids 257-264 of SEQ ID NO: 28.

In some embodiments, the extracellular region comprises a signal peptide. In some embodiments, the signal peptide of the human or chimeric IL10R (e.g., IL10RA) described herein has a sequence that is at least or about 50%, 60%, 70%, 80%, 90%, 95%, 99% identical to the transmembrane region of human IL10R (e.g., IL10RA). In some embodiments, the signal peptide of the human or chimeric IL10R (e.g., IL10RA) has a sequence that has at least 1, 2, 3, 4, 5, 6, 7, 8, 9, 10, 15, or 20 amino acids (e.g., contiguously or non-contiguously) that are identical to human IL10R. In some embodiments, the signal peptide of the human or chimeric IL10R (e.g., IL10RA) comprises a sequence that is the entire of a part of amino acids 1-21 of SEQ ID NO: 28.

The genome of the genetically modified animal can comprise a replacement at an endogenous IL10 gene locus of a sequence encoding a region of endogenous IL10 with a sequence encoding a corresponding region of human IL10. In some embodiments, the sequence that is replaced is any sequence within the endogenous IL10 gene locus, e.g., exon 1, exon 2, exon 3, exon 4, exon 5, 5′-UTR, 3′UTR, the first intron, the second intron, the third intron, or the fourth intron, etc. In some embodiments, the sequence that is replaced is within the regulatory region of the endogenous IL10 gene. In some embodiments, the sequence that is replaced is within the regulatory region of the human IL10 gene.

Because human protein and non-human protein sequences, in many cases, are different, antibodies that bind to human protein will not necessarily have the same binding affinity with non-human protein or have the same effects to non-human protein. Therefore, the genetically modified animal expressing human IL10 and the genetically modified animal having a human or a humanized extracellular region of IL10R (e.g., IL10RA) can be used to better evaluate the effects of anti-IL10 or anti-IL10R antibodies in an animal model. In some embodiments, the genome of the genetically-modified animal comprises a sequence encoding an amino acid sequence that corresponds to part or the entire sequence of exon 1, exon 2, exon 3, exon 4, exon 5, and/or exon 6 of human IL10R (e.g., IL10RA), part or the entire sequence of the extracellular region, the transmembrane region, and/or the cytoplasmic region of human IL10R (e.g., IL10RA) (with or without signal peptide), or part or the entire sequence of amino acids 1-264 of SEQ ID NO: 28.

In some embodiments, the non-human animal can have, at an endogenous IL10R gene (e.g., IL10RA) locus, a nucleotide sequence encoding a chimeric human/non-human IL10R (e.g., IL10RA) polypeptide, wherein a human portion of the chimeric human/non-human IL10R (e.g., IL10RA) polypeptide comprises a portion of human IL10R (e.g., IL10RA) extracellular region, transmembrane region, and/or cytoplasmic region, and wherein the animal expresses a functional IL10R (e.g., IL10RA) on a surface of a cell of the animal. The human portion of the chimeric human/non-human IL10R (e.g., IL10RA) polypeptide can comprise a portion of exon 1, exon 2, exon 3, exon 4, exon 5, and/or exon 6 of human IL10R (e.g., IL10RA). In some embodiments, the human portion of the chimeric human/non-human IL10RA polypeptide can comprise a sequence that is at least 80%, 85%, 90%, 95%, or 99% identical to amino acids 1-264 of SEQ ID NO: 28.

In some embodiments, the non-human portion of the chimeric human/non-human IL10RA polypeptide comprises the cytoplasmic region of an endogenous non-human IL10RA polypeptide. There may be several advantages that are associated with the cytoplasmic regions of an endogenous non-human IL10RA polypeptide. For example, once IL10 binds to IL10R, they can properly transmit extracellular signals into the cells and regulate the downstream pathway.

In some embodiments, the chimeric human/non-human IL10RA polypeptide comprises a transmembrane region from human IL10RA. The human IL10RA transmembrane region may provide several advantages. For example, as the sequence identity of the human IL10RA transmembrane region and mouse IL10RA transmembrane region is relatively low, the mouse IL10RA transmembrane region may not properly interact with the humanized extracellular region. In addition, the mouse IL10RA transmembrane region may also affect membrane localization of the chimeric human/mouse IL10RA.

In some embodiments, the humanized IL10RA locus lacks a human IL10RA 5′-UTR. In some embodiment, the humanized IL10RA locus comprises a rodent (e.g., mouse) 5′-UTR. In some embodiments, the humanization comprises a human 3′-UTR. In appropriate cases, it may be reasonable to presume that the mouse and human IL10RA genes appear to be similarly regulated based on the similarity of their 5′-flanking sequence. As shown in the present disclosure, humanized IL10RA mice that comprise a replacement at an endogenous mouse IL10RA locus, which retain mouse regulatory elements but comprise a humanization of IL10RA encoding sequence, do not exhibit obvious pathologies. Both genetically modified mice that are heterozygous or homozygous for humanized IL10RA are grossly normal.

In some embodiments, the humanized IL10 locus has a human IL10 5′-UTR or an endogenous IL10 5′-UTR. In some embodiment, the humanized IL10 locus comprises a rodent (e.g., mouse) 5′-UTR. In some embodiments, the humanization comprises a human 3′-UTR or an endogenous 3′-UTR. In appropriate cases, it may be reasonable to presume that the mouse and human IL10 genes appear to be similarly regulated based on the similarity of their 5′-flanking sequence. As shown in the present disclosure, humanized IL10 mice that comprise a replacement at an endogenous mouse IL10 locus, which has mouse or human regulatory elements, do not exhibit obvious pathologies. Both genetically modified mice that are heterozygous or homozygous for humanized IL10 are grossly normal.

The present disclosure further relates to a non-human mammal generated through the method mentioned above. In some embodiments, the genome thereof contains human gene(s). In some embodiments, the non-human mammal is a rodent, and preferably, the non-human mammal is a mouse.

In some embodiments, the non-human mammal expresses a protein encoded by a humanized IL10R or IL10 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL10RA or IL10 in the genome of the animal.

In some embodiments, the non-human mammal comprises the genetic construct as described herein. In some embodiments, a non-human mammal expressing human or humanized IL10RA or IL10 is provided. In some embodiments, the tissue-specific expression of human or humanized IL10RA or IL10 protein is provided.

In some embodiments, the expression of human or humanized IL10RA or IL10 in a genetically modified animal is controllable, as by the addition of a specific inducer or repressor sub stance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human or humanized IL10R (e.g., IL10RA) or IL10 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL10R (e.g., IL10RA) or IL10 protein.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) amino acid sequence from an endogenous non-human IL10RA or IL10 locus.

### Vectors

The present disclosure relates to a targeting vector, comprising: a) a DNA fragment homologous to the 5′ end of a region to be altered (5′ arm), which is selected from the IL10RA or IL10 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL10RA or IL10 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000067.6; c) the DNA fragment homologous to the 3′ end of the region to be altered (3′ arm) is selected from the nucleotide sequences that have at least 90% homology to the NCBI accession number NC_000067.6.

In some embodiments, a) the DNA fragment homologous to the 5′ end of a region to be altered (5′ arm) is selected from the nucleotides from the position 131016009 to the position 131019911 of the NCBI accession number NC_000067.6; c) the DNA fragment homologous to the 3′ end of the region to be altered (3′ arm) is selected from the nucleotides from the position 131025304 to the position 131029830 of the NCBI accession number NC_000067.6.

In some embodiments, a) the DNA fragment homologous to the 5′ end of a conversion region to be altered (5′ arm) is selected from the nucleotide sequences that have at least 90% homology to the NCBI accession number NC_000075.6; c) the DNA fragment homologous to the 3′ end of the region to be altered (3′ arm) is selected from the nucleotide sequences that have at least 90% homology to the NCBI accession number NC_000075.6.

In some embodiments, a) the DNA fragment homologous to the 5′ end of a region to be altered (5′ arm) is selected from the nucleotides from the position 45274777 to the position 45269073 of the NCBI accession number NC_000075.6; c) the DNA fragment homologous to the 3′ end of the region to be altered (3′ arm) is selected from the nucleotides from the position 45260102 to the position 45253456 of the NCBI accession number NC_000075.6.

In some embodiments, the length of the selected genomic nucleotide sequence in the targeting vector can be about or at least 3 kb, 4 kb, 5 kb, 6 kb, 7 kb, 8 kb, 9 kb or 10 kb.

In some embodiments, the region to be altered is exon 1, exon 2, exon 3, exon 4, and/or exon 5 of IL10 gene (e.g., a portion of exon 1, exon 2, exon 3, exon 4 and a portion of exon 5 of mouse IL10 gene).

In some embodiments, the region to be altered is exon 1, exon 2, exon 3, exon 4, exon 5, exon 6, and/or exon 7 of IL10RA gene (e.g., a portion of exon 1, exon 2, exon 3, exon 4, exon 5, and a portion of exon 6 of mouse IL10RA gene).

The targeting vector can further include a selected gene marker.

In some embodiments, the sequence of the 5′ arm is shown in SEQ ID NO: 5; and the sequence of the 3′ arm is shown in SEQ ID NO: 6.

In some embodiments, the sequence of the 5′ arm is shown in SEQ ID NO: 29; and the sequence of the 3′ arm is shown in SEQ ID NO: 30.

In some embodiments, the sequence is derived from human (e.g., 206772435-206768636 of NC_000001.11). For example, the target region in the targeting vector is a part or entirety of the nucleotide sequence of a human IL10, preferably exon 1, exon 2, exon 3, exon 4, and/or exon 5 of the human IL10. In some embodiments, the nucleotide sequence of the humanized IL10 encodes the entire or a part of human IL10 protein (e.g., SEQ ID NO: 4).

In some embodiments, the sequence is derived from human (e.g., 117986468-117995692 of NC_000011.10). For example, the target region in the targeting vector is a part or entirety of the nucleotide sequence of a human IL10RA, preferably exon 1, exon 2, exon 3, exon 4, exon 5, and/or exon 6 of the human IL10RA. In some embodiments, the nucleotide sequence of the humanized IL10RA encodes the entire or a part of human IL10RA protein (e.g., SEQ ID NO: 28).

In some embodiments, the target region is derived from human. In some embodiments, the target region is a part or entirety of the nucleotide sequence of a humanized IL10RA. In some embodiments, the nucleotide sequence is shown as one or more of exon 1, exon 2, exon 3, exon 4, exon 5, exon 6, and/or exon 7 of the human IL10RA. In some embodiments, the target region is a part or entirety of the nucleotide sequence of a humanized IL10. In some embodiments, the nucleotide sequence is shown as one or more of exon 1, exon 2, exon 3, exon 4, and/or exon 5 of the human IL10.

In some embodiments, the nucleotide sequence of the human IL10RA encodes the human IL10RA protein with the NCBI accession number NP_001549.2 (SEQ ID NO: 28). In some emboldens, the nucleotide sequence of the human IL10RA is selected from the nucleotides from the position 117986468 to the position 117995692 of NC_000011.10.

In some embodiments, the nucleotide sequence of the human IL10 encodes the human IL10 protein with the NCBI accession number NP_000563.1 (SEQ ID NO: 4). In some emboldens, the nucleotide sequence of the human IL10 is selected from the nucleotides from the position 206772435 to the position 206768636 of NC_000001.11.

The disclosure also relates to a cell comprising the targeting vectors as described herein.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL10RA or IL10 gene locus, a sequence encoding a region of an endogenous IL10RA or IL10 with a sequence encoding a corresponding region of human or chimeric IL10RA or IL10. In some embodiments, the replacement occurs in a germ cell, a somatic cell, a blastocyst, or a fibroblast, etc. The nucleus of a somatic cell or the fibroblast can be inserted into an enucleated oocyte.

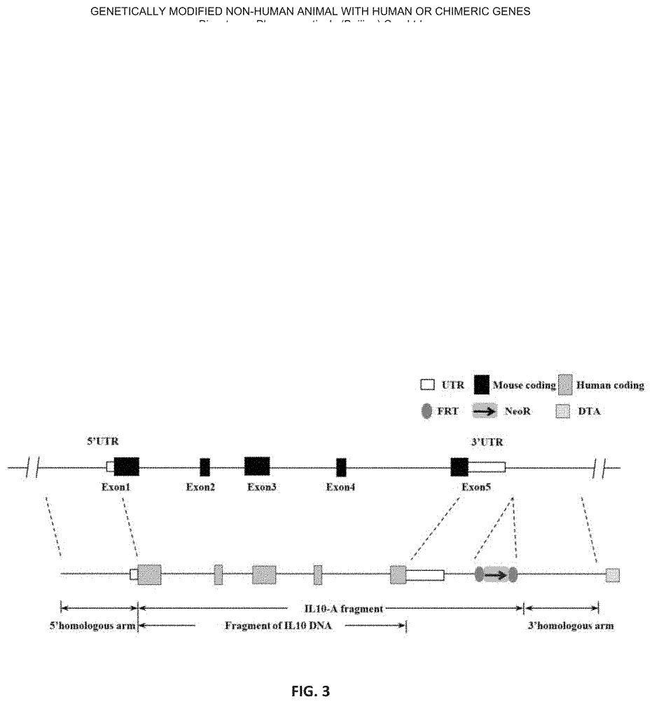

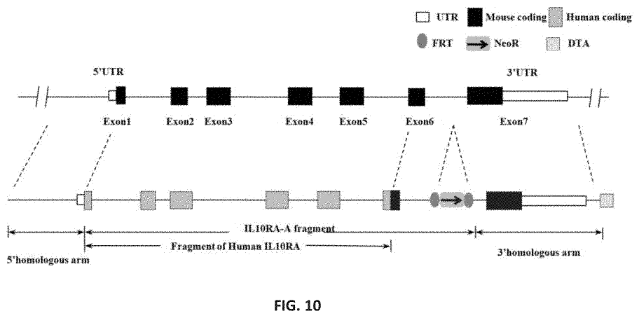

FIG. 10 shows a humanization strategy for a mouse IL10RA locus. In FIG. 10, the targeting strategy involves a vector comprising the 5′ end homologous arm, human IL10RA gene fragment, 3′ homologous arm. The process can involve replacing endogenous IL10RA sequence with human sequence by homologous recombination. FIG. 3 shows a humanization strategy for a mouse IL10 locus. In FIG. 3, the targeting strategy involves a vector comprising the 5′ end homologous arm, human IL10 gene fragment, 3′ homologous arm. The process can involve replacing endogenous IL10 sequence with human sequence by homologous recombination. In some embodiments, the cleavage at the upstream and the downstream of the target site (e.g., by zinc finger nucleases, TALEN or CRISPR) can result in DNA double strand break, and the homologous recombination is used to replace endogenous IL10RA or IL10 sequence with human IL10RA or IL10 sequence.

Thus, in some embodiments, the methods for making a genetically modified, humanized animal, can include the step of replacing at an endogenous IL10RA locus (or site), a nucleic acid encoding a sequence encoding a region of endogenous IL10RA with a sequence encoding a corresponding region of human IL10RA. The sequence can include a region (e.g., a part or the entire region) of exon 1, exon 2, exon 3, exon 4, exon 5, exon 6, and/or exon 7 of a human IL10RA gene. In some embodiments, the sequence includes a portion of exon 1, exon 2, exon 3, exon 4, exon 5, and/or a portion of exon 6 of a human IL10RA gene (e.g., amino acids 1-264 of SEQ ID NO: 28). In some embodiments, the region is located within the extracellular region, the transmembrane region, and/or the cytoplasmic region of a human IL10RA. In some embodiments, the endogenous IL10RA locus is exon 1, exon 2, exon 3, exon 4, exon 5, and/or exon 6 of mouse IL10RA.

Thus, in some embodiments, the methods for making a genetically modified, humanized animal, can include the step of replacing at an endogenous IL locus (or site), a nucleic acid encoding a sequence encoding a region of endogenous IL10 with a sequence encoding a corresponding region of human IL10. The sequence can include a region (e.g., a part or the entire region) of exon 1, exon 2, exon 3, exon 4, and/or exon 5 of a human IL10 gene. In some embodiments, the sequence includes a portion of exon 1, exon 2, exon 3, exon 4, and/or a portion of exon 5 of a human IL10 gene (e.g., amino acids 1-178 of SEQ ID NO: 4). In some embodiments, the endogenous IL10 locus is exon 1, exon 2, exon 3, exon 4 and/or exon 5 of mouse IL10.

In some embodiments, the methods of modifying an IL10RA or IL10 locus of a mouse to express a chimeric human/mouse IL10RA or IL10 peptide can include the steps of replacing at the endogenous mouse IL10RA or IL10 locus a nucleotide sequence encoding a mouse IL10RA or IL10 with a nucleotide sequence encoding a human IL10RA or IL10, thereby generating a sequence encoding a chimeric human/mouse IL10RA or IL10.

In some embodiments, the nucleotide sequence encoding the chimeric human/mouse IL10RA can include a first nucleotide sequence encoding the extracellular region, the transmembrane region, and a portion of the cytoplasmic region of human IL10RA (with or without the mouse or human signal peptide sequence); and/or a second nucleotide sequence encoding a region of the cytoplasmic region of mouse IL10RA.

In some embodiments, the nucleotide sequences as described herein do not overlap with each other (e.g., the first nucleotide sequence, and/or the second nucleotide sequence do not overlap). In some embodiments, the amino acid sequences as described herein do not overlap with each other.

The present disclosure further provides a method for establishing an IL10RA or IL10 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 or BALB/c mouse).

In some embodiments, the non-human mammal in step (c) is a female with pseudo pregnancy (or false pregnancy).

In some embodiments, the fertilized eggs for the methods described above are C57BL/6 or BALB/c fertilized eggs. Other fertilized eggs that can also be used in the methods as described herein include, but are not limited to, FVB/N fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically-modified animals that express human or humanized IL10RA and/or IL10 protein, e.g., in a physiologically appropriate manner, provide a variety of uses that include, but are not limited to, developing therapeutics for human diseases and disorders, and assessing the toxicity and/or efficacy of these human therapeutics in the animal models.

In various aspects, genetically modified animals are provided that express human or humanized IL10R and/or IL10, which are useful for testing agents that can decrease or block the interaction between IL10R complex and IL10 or the interaction between IL10R complex and other IL10R ligands, testing whether an agent can increase or decrease the immune response, and/or determining whether an agent is an IL10 pathway agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In one aspect, the disclosure also provides methods of determining effectiveness of an IL10 pathway agonist (e.g., an anti-IL10R or an anti-IL10 antibody) for reducing inflammation. The methods involve administering the IL10 pathway agonist to the animal described herein, wherein the animal has an inflammation; and determining the inhibitory effects of the agent to the reduction of inflammation.

In one aspect, the disclosure also provides methods of determining effectiveness of an IL10 pathway agonist (e.g., an anti-IL10R or anti-IL10 antibody) for treating an autoimmune disorder (e.g., asthma). The methods involve administering the IL10 pathway agonist to the animal described herein, wherein the animal has an autoimmune disorder; and determining the inhibitory effects of the agent.

In one aspect, the disclosure also provides methods of determining effectiveness of an IL10 pathway antagonist (e.g., an anti-IL10R or an anti-IL10 antibody) for treating an infectious disease (e.g., tuberculosis). The methods involve administering the IL10 pathway antagonist to the animal described herein, wherein the animal has an infectious disease (e.g., tuberculosis); and determining the inhibitory effects of the IL10 pathway antagonist. In some embodiments, the infectious disease is caused by pathogens, e.g., Leishmania donovani, Mycobacterium tuberculosis, Trypanosoma cruzi, or Coxiella burnetii.

In one aspect, the disclosure also provides methods of determining effectiveness of an IL10 pathway antagonist (e.g., an anti-IL10R or anti-IL10 antibody) for treating cancer. The methods involve administering the IL10 pathway antagonist to the animal described herein, wherein the animal has a tumor; and determining the inhibitory effects of the IL10 pathway antagonist to the tumor. In some embodiments, the tumor comprises one or more cancer cells that are injected into the animal. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the anti-IL10R antibody or anti-IL10 antibody prevents IL10 from binding to IL10R. In some embodiments, the anti-IL10R antibody or anti-IL10 antibody cannot prevent IL10 from binding to IL10R (e.g., endogenous, human, or humanized IL10R).

In some embodiments, the genetically modified animals can be used for determining whether an anti-IL10R antibody is an IL10R agonist or antagonist. In some embodiments, the genetically modified animals can be used for determining whether an anti-IL10 antibody is an IL10 agonist or antagonist. In some embodiments, the methods as described herein are also designed to determine the effects of the agent (e.g., anti-IL10R or anti-IL10 antibodies) on IL10R and/or IL10, e.g., whether the agent can upregulate the immune response or downregulate immune response. In some embodiments, the genetically modified animals can be used for determining the effective dosage of a therapeutic agent for treating a disease in the subject, e.g., a cancer, an infectious disease, or an immune disorder (e.g., an autoimmune disease).

In some embodiments, the inhibitory effects of treating inflammation are evaluated by serum IgE levels; pathological lung histology features; number of leukocytes (CD45+ cells), eosinophils (Eos) or neutrophils in bronchoalveolar lavage fluid (BALF); or ratio of eosinophils or neutrophils cells in CD45+ cells in bronchoalveolar lavage fluid (BALF).

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-IL10R or anti-IL10 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, Melanoma and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the antibody is designed for treating various autoimmune diseases (e.g., asthma, systemic lupus erythematosus (SLE), rheumatoid arthritis (RA), Sjogren's syndrome (SS), multiple sclerosis (MS), Crohn's disease (CD), inflammatory bowel disease (IBD), or psoriasis) or allergy. Thus, the methods as described herein can be used to determine the effectiveness of an antibody in inhibiting immune response.

The present disclosure also provides methods of determining toxicity of an antibody (e.g., anti-IL10R antibody, anti-IL10RA antibody, or anti-IL10 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL10R or IL10 gene function, human IL10R or IL10 antibodies, drugs for human IL10R or IL10 targeting sites, the drugs or efficacies for human IL10R or IL10 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL10RA or IL10 gene humanized non-human animal prepared by the methods described herein, the IL10RA or IL10 gene humanized non-human animal described herein, the double- or multi-humanized non-human animal generated by the methods described herein (or progeny thereof), a non-human animal expressing the human or humanized IL10RA or IL10 protein, or the tumor-bearing or inflammatory animal models described herein. In some embodiments, the TCR-T, CAR-T, and/or other immunotherapies can treat the IL10/IL10R pathway-associated diseases described herein. In some embodiments, the TCA-T, CAR-T, and/or other immunotherapies provides an evaluation method for treating the IL10/IL10R pathway-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric IL10RA gene and a sequence encoding one or more additional human or chimeric protein (e.g., IL10). Alternatively, the animal can comprise a human or chimeric IL10 gene and a sequence encoding one or more additional human or chimeric protein (e.g., IL10RA).

In some embodiments, the additional human or chimeric protein can be IL10, IL10RA, Interleukin 33 (IL33), Interleukin 3 (IL3), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (TNFRSF4 or OX40), CD47 or SIRPa.

The methods of generating genetically modified animal model with two or more human or chimeric genes (e.g., humanized genes) can include the following steps:

(a) using the methods of introducing human IL10RA gene or chimeric IL10RA gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL10, IL33, IL3, PD-1, CTLA-4, LAG-3, BTLA, PD-L1, CD27, CD28, TIGIT, TIM-3, GITR, OX40, CD137, CD47, or SIRPa. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/117984, PCT/CN2017/120388; each of which is incorporated herein by reference in its entirety.

Similarly, the methods of generating genetically modified animal model can include the following steps:

(a) using the methods of introducing human IL10 gene or chimeric IL10 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, the humanization is directly performed on a genetically modified animal having a human or chimeric IL10, IL10R, IL10RA, IL10Rb, IL33, IL3, PD-1, CTLA-4, BTLA, PD-L1, CD27, CD28, TIGIT, TIM-3, GITR, CD137, OX40, CD47 or SIRPa gene.

In some embodiments, the IL10RA humanization is directly performed on a genetically modified animal having a human or chimeric IL10. In some embodiments, the IL10 humanization is directly performed on a genetically modified animal having a human or chimeric IL10RA.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL10R antibody and an additional therapeutic agent for the treatment. The methods include administering the anti-IL10R antibody (e.g., an anti-IL10RA antibody) and/or the anti-IL10 antibody, and the additional therapeutic agent to the animal, wherein the animal has a tumor; and determining the inhibitory effects of the combined treatment to the tumor. In some embodiments, the additional therapeutic agent is an antibody that specifically binds to IL10, IL10R, IL10RA, IL10Rb, IL33, IL3, PD-1, CTLA-4, BTLA, PD-L1, CD27, CD28, TIGIT, TIM-3, GITR, CD137, OX40, CD47 or SIRPa. In some embodiments, the additional therapeutic agent is an anti-CTLA4 antibody (e.g., ipilimumab), an anti-CD20 antibody (e.g., rituximab), an anti-EGFR antibody (e.g., cetuximab), and an anti-CD319 antibody (e.g., elotuzumab), or anti-PD-1 antibody (e.g., nivolumab).

### EXAMPLES

The invention is further described in the following examples, which do not limit the scope of the invention described in the claims.

### Materials and Methods

The following materials were used in the following examples.

C57BL/6 mice and Flp transgenic mice were purchased from the China Food and Drugs Research Institute National Rodent Experimental Animal Center.

Cas9 mRNA was obtained from SIGMA. The catalog number is CAS9MRNA-1EA.

UCA kit was obtained from Beijing Biocytogen Co., Ltd. The catalog number is BCG-DX-001.

Ambion™ in vitro transcription kit (MEGAshortscript™ Kit) was purchased from Thermo Fisher Scientific. The catalog number is AM1354.

Lipopolysaccharides from Escherichia coli O111:B4 (LPS) was purchased from Merck. The catalog number is L2630.

SacI, PstI, SpeI, HindIII, XbaI, ScaI, BamHI and SspI restriction enzymes were purchased from NEB with catalog numbers: R3156M, R3140M, R3133M, R3104M, R3122M, R3122M, R3136L and R3132L, respectively.

ELISA MAX™ Deluxe Set Mouse IL-10 (MouseIL-10 ELISA Kit) was purchased from BioLegend, Inc. (Catalog Number: 431414).

ELISA MAX™ Deluxe Set Human IL-10 (HumanIL-10 ELISA Kit) was purchased from BioLegend, Inc. (Catalog Number: 430604).

### Example 1: Mice with Humanized IL-10 Gene

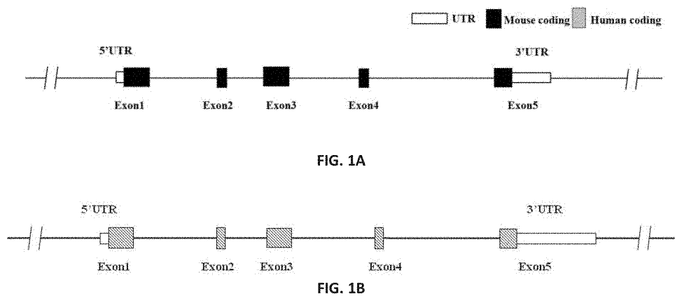

The mouse IL-10 gene (NCBI Gene ID: 16153, Primary source: MGI: 96537, UniProt ID: P18893) is located at 131019845 to 131024974 of chromosome 1 (NC_000067.6), and the human IL10 gene (NCBI Gene ID: 3586, Primary source: HGNC: 5962, UniProt ID: P22301) is located at 206767602 to 206772494 of chromosome 1 (NC_000001.11). FIG. 1A shows the mouse transcript NM_010548.2 (SEQ ID NO: 1) and the corresponding protein sequence NP_034678.1 (SEQ ID NO: 2). FIG. 1B shows the human transcript NM_000572.3 (SEQ ID NO: 3) and the corresponding protein sequence NP_000563.1 (SEQ ID NO: 4).

For the purpose of the experiments, a gene sequence encoding the human IL-10 protein can be introduced into the endogenous mouse IL-10 locus, such that the mouse can express a human or humanized IL-10 protein. Mouse cells can be modified by various gene editing techniques, for example, a nucleic acid sequence comprising human IL-10 gene or the coding sequence of human IL10 protein can be inserted into the mouse endogenous IL-10 locus. Auxiliary sequences (e.g., stop codons) can be added after the inserted sequence, or other methods (e.g., flipping or knocking out the endogenous IL-10 gene sequence) can be applied so that the mouse endogenous IL-10 protein cannot be expressed normally after the insertion site. Alternatively, in situ replacement can also be used, that is, the nucleotide sequence at the endogenous IL-10 gene locus is directly substituted (e.g., replaced) with the human IL-10 gene sequence. This example used in situ replacement to illustrate humanization of the IL-10 gene.

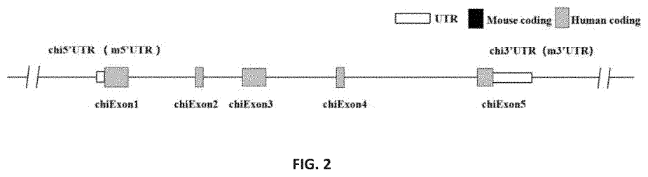

Specifically, a nucleic acid sequence of mouse IL-10 gene can be replaced with a nucleic acid sequence of human IL-10 gene at the endogenous IL-10 locus by gene editing technologies. For example, under control of a mouse IL-10 regulatory element, a sequence of about 4375 bp spanning from exon 1 (including a part of exon 1) to exon 5 (including a part of exon 5) of mouse IL-10 gene was replaced with a corresponding human DNA sequence to obtain a humanized IL-10 locus, thereby humanizing mouse IL-10 gene (shown in FIG. 2). The humanization allowed the mouse to transcribe a chimeric IL-10 mRNA and express human IL-10 protein.

As shown in the schematic diagram of the targeting strategy in FIG. 3, a targeting vector was constructed, which contained the homologous arm sequences upstream and downstream of the endogenous mouse IL-10, and a “IL-10-A fragment” containing the human IL-10 gene sequence. The upstream homologous arm (5′ homologous arm, SEQ ID NO: 5) contains a sequence 3903 bp upstream of the 5′UTR (including the 5′ UTR), and is identical to the nucleic acids 131016009-131019911 of NCBI Reference Number NC_000067.6. The downstream homologous arm (3′ homologous arm, SEQ ID NO: 6) contains a sequence 4527 bp downstream of the 3′UTR, and is identical to nucleic acids 131025304-131029830 of NCBI Reference Number NC_000067.6. The IL-10-A fragment contains a human IL-10 gene fragment starting within exon 1 and ending within exon 5 of the human IL-10 gene. The human IL-10 gene fragment is identical to nucleic acids 206772435-206768636 of NCBI Reference Number NC_000001.11.

The 5′ end of human IL-10 gene fragment is directly connected to the 5′ homologous arm. The connection between the 3′ end of the human IL-10 gene fragment and the mouse IL-10 gene locus was designed as 5′-CATCAACTACATAGAAGCCTACATGACAATGAAGATACGAAACTGAAACACCTGCA GTGTGTATTGAGTCTGCTGGACTCCAGGACCTAGACAGAG-3′ (SEQ ID NO: 52), wherein the “A” of sequence “CTGA” is the last nucleotide of the human sequence, and the first “A” of sequence “AACA” is the first nucleotide of the mouse sequence.

The mRNA sequence of the engineered mouse IL-10 after humanization and its encoded protein sequence are shown in SEQ ID NO: 7 and SEQ ID NO: 4 (same as the human IL-10 protein sequence), respectively. The targeting vector also included an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the mouse IL-10 gene locus was designed as 5′-AACTCTGAGACGAAATGTTGGGCTCGAGGTCGACGGTATCGATAAGCTTGATATCG AATTCCGAAGTTCCTATTC-3′ (SEQ ID NO: 8), wherein the last “G” of sequence “TGGG” is the last nucleotide of the mouse sequence, and the first C of sequence “CTCG” is the first nucleotide of the Neo cassette. The connection between the 3′ end of the Neo cassette with the mouse IL-10 gene locus was designed as 5′-TATTCTCTAGAAAGTATAGGAACTTCATCAGTCAGGTACATAATGGTGGATCCGTTA AAAATGGAAGCTAGGGGCAG-3′ (SEQ ID NO: 9), wherein the last “C” of sequence “ATCC” is the last nucleotide of the Neo cassette, and the “G” of sequence “GTTA” is the first nucleotide of the mouse sequence. In addition, a coding gene with a negative selectable marker (a gene encoding diphtheria toxin A subunit (DTA)) was also inserted downstream of the 3′ homologous arm of the targeting vector.

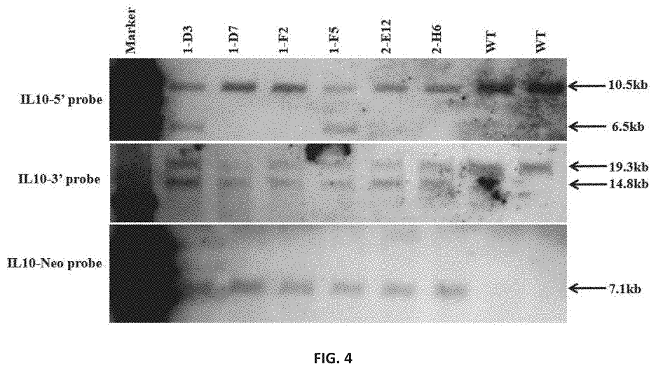

The targeting vector was constructed by restriction enzyme digestion and ligation. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot (digested with StuI, BamHI, and HindIII, respectively, and then hybridized with 3 probes) to screen out correct positive clone cells. As shown in FIG. 4, the results indicated that among the 6 positive clones confirmed by PCR, 1-D3 and 1-F5 were positive heterozygous clones without random insertions.

The following primers were used in PCR:

IL-10-F1:
(SEQ ID NO: 10)
5′-TGCCCAGGTCACTAAAGCAGGTTA-3′,
IL-10-R1:
(SEQ ID NO: 11)
5′-GAGCGCCAGCAGGATCTTATAAGTT-3′;
IL-10-F2:
(SEQ ID NO: 12)
5′-CGCATTGTCTGAGTAGGTGTC-3′,
IL-10-R2:
(SEQ ID NO: 13)
5′-GTCTCCAGAGTCACCACATGTGTTG-3′.

The following probes were used in Southern Blot assays:

IL-10-5′ Probe:
F:
(SEQ ID NO: 14)
5′-CAAAACGGTTATCTCTGAGTAGCCCGA-3′,
R:
(SEQ ID NO: 15)
5′-GGTGGCTTCTCTGTATCCAACCCC-3′;
IL-10-3′ Probe:
F:
(SEQ ID NO: 16)
5′-GGGCAGGAGTTCCAGCCTGAATAG-3′,
R:
(SEQ ID NO: 17)
5′-AGCTTCTGGAAATTTAAGCCCACCCAT-3′;
IL-10-Neo Probe:
F:
(SEQ ID NO: 18)
5′-GGATCGGCCATTGAACAAGATGG-3′,
R:
(SEQ ID NO: 19)
5′-CAGAAGAACTCGTCAAGAAGGCG-3′.

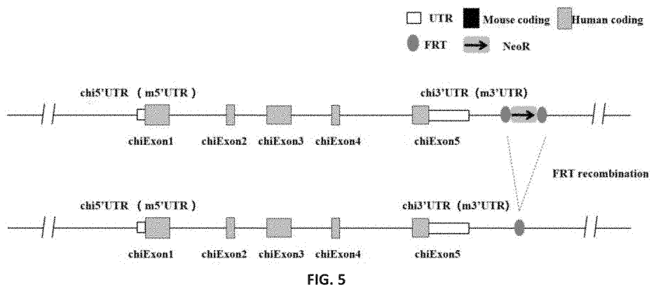

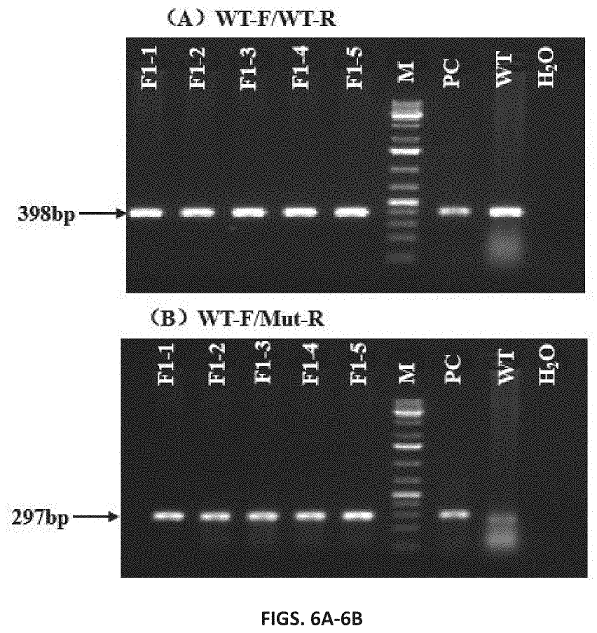

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp mice to remove the positive selectable marker gene (FIG. 5), and then the humanized IL-10 homozygous mice expressing humanized IL-10 protein were obtained by breeding with each other. The genotype of the progeny mice can be identified by PCR. The identification results of exemplary F1 generation mice (Neo cassette-removed) are shown in FIGS. 6A-6B, and mice labelled F1-1, F1-2, F1-3, F1-4, and F1-5 were identified as positive heterozygous clones. The following primers were used in the PCR or RT-PCR identification:

IL-10-WT-F:
(SEQ ID NO: 20)
5′-GGTTTAGAAGAGGGAGGAGGAGCC-3′;
IL-10-WT-R:
(SEQ ID NO: 21)
5′-TGCAGTTCCATCAGAATGCATATTTCAG-3′;
IL-10-WT-F:
SEQ ID NO: 20;
IL-10-Mut-R:
(SEQ ID NO: 22)
5′-AGATCTCGAAGCATGTTAGGCAGG-3′;
IL-10-Frt-F:
(SEQ ID NO: 23)
5′-GCTGAAACTCTGAGACGAAATGTT-3′;
IL-10-Frt-R:
(SEQ ID NO: 24)
5′-CTTGAAGCAACCACTGACACATTAG-3′;
hIL10-RT-PCR-F3:
(SEQ ID NO: 54)
5′-AGGGCACCCAGTCTGAGAACAG-3′;
hIL10-RT-PCR-R3:
(SEQ ID NO: 55)
5′-GCGCCTTGATGTCTGGGTCTTGG-3′;
hIL10-RT-PCR-F4:
(SEQ ID NO: 56)
5′-CAGCTGGACAACTTGTTGTTAAAGGAG-3′;
hIL10-RT-PCR-R4:
(SEQ ID NO: 57)
5′-ACTCATGGCTTTGTAGATGCCTTTC-3′;
mIL10-RT-PCR-F3:
(SEQ ID NO: 58)
5′-TGACTGGCATGAGGATCAGCAGG-3′;
mIL10-RT-PCR-R3:
(SEQ ID NO: 59)
5′-CTCCTTGATTTCTGGGCCATGC-3′;
mIL10-RT-PCR-F4:
(SEQ ID NO: 60)
5′-GCTGGACAACATACTGCTAACCGAC-3′;
mIL10-RT-PCR-R4:
(SEQ ID NO: 61)
5′-ATTCATGGCCTTGTAGACACCTTGG-3′;
GAPDH-F:
(SEQ ID NO: 62)
5′-TCACCATCTTCCAGGAGCGAGA-3′;
GAPDH-R:
(SEQ ID NO: 63)
5′-GAAGGCCATGCCAGTGAGCTT-3′.

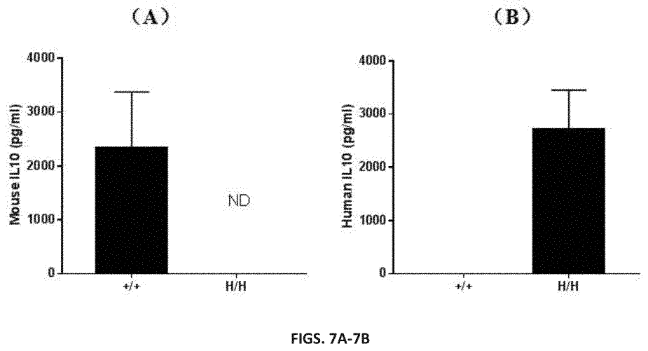

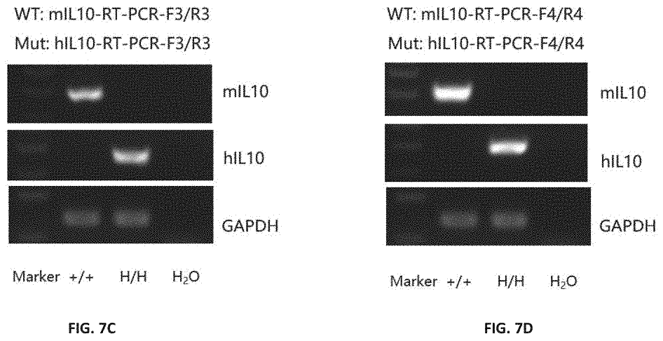

The results indicated that this method can be used to construct genetically engineered IL-10 mice and the genetic modification can be stably passed to the next generation without random insertions. The expression of human IL-10 protein in positive mice can be confirmed by routine detection methods, e.g., ELISA. Specifically, three 6-7 weeks old wild-type mice and three IL-10 gene humanized homozygous mice were selected and injected intraperitoneally with 20 μg of lipopolysaccharide (LPS). Serum was collected 1 hour later, and mouse or human IL-10 protein levels were measured using ELISA kits (Mouse IL-10 ELISA Kit and Human IL-10 ELISA Kit, respectively). The detection results are shown in FIGS. 7A-7B. As shown in FIG. 7A, expression of mouse IL-10 protein was only detected in wild-type C57BL/6 mice. As shown in FIG. 7B, expression of human IL-10 protein was only detected in IL-10 gene humanized mice. FIGS. 7C-7D are results from RT-PCR. It confirms that the expression of human IL-10 protein was only detected in IL-10 gene humanized mice.

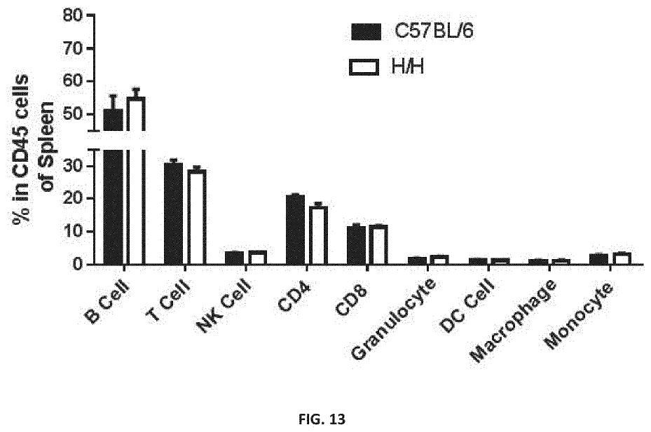

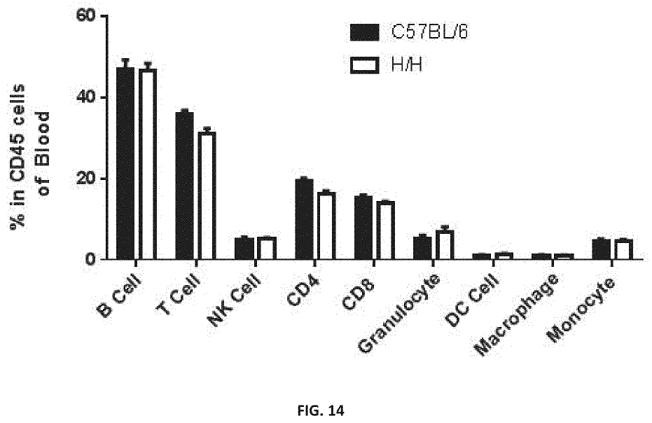

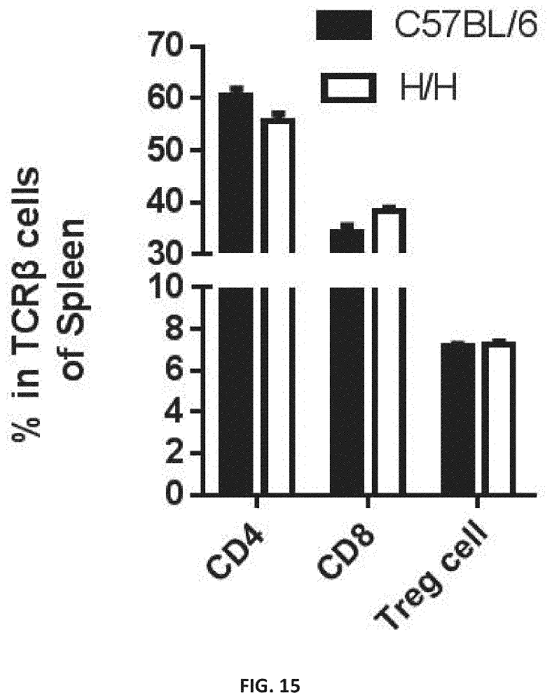

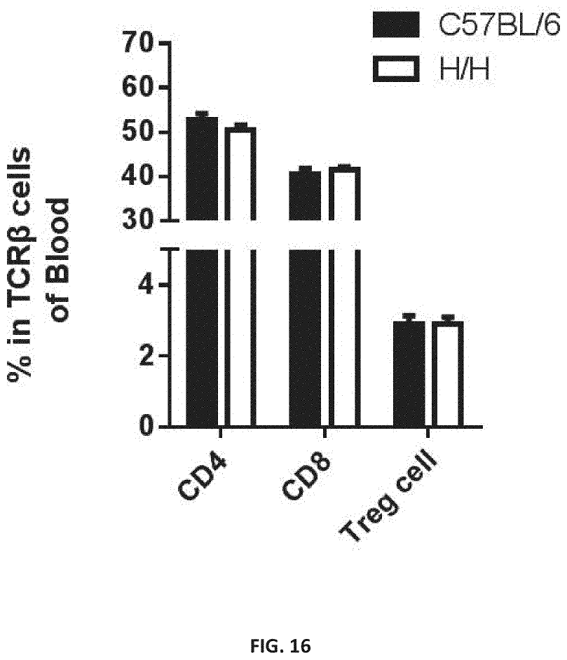

Furthermore, the immune cells and T cell subsets in the spleen and peripheral blood of the mice were also tested, and no significant differences were detected as compared to the wild type mice (FIGS. 13-16). The results indicate that differentiation of the B cells, T cells, NK cells, CD4+ T cells (CD4), CD8+ T cells (CD8), granulocytes, DC cells, macrophages, and monocytes was not affected.

### Example 2: Mice with Humanized IL-10RA Gene

The mouse IL-10RA gene (NCBI Gene ID: 16154, Primary source: MGI: 96538, UniProt ID: Q61727) is located at 45253837 to 45269149 of chromosome 9 (NC_000075.6), and the human IL-10RA gene (NCBI Gene ID: 3587, Primary source: HGNC: 5964, UniProt ID: Q13651) is located at 117986391 to 118001483 of chromosome 11 (NC_000011.10). FIG. 8A shows the mouse transcript NM_008348.3 (SEQ ID NO: 25) and the corresponding protein sequence NP_032374.1 (SEQ ID NO: 26). FIG. 1B shows the human transcript NM_001558.3 (SEQ ID NO: 27) and the corresponding protein sequence NP_001549.2 (SEQ ID NO: 28).

For the purpose of the experiments, a gene sequence encoding the human IL-10RA protein can be introduced into the endogenous mouse IL-10RA locus, such that the mouse can express a human or humanized IL-10RA protein. Mouse cells can be modified by various gene editing techniques, for example, a nucleic acid sequence comprising human IL-10RA gene or the coding sequence of human IL-10RA protein can be inserted into the mouse endogenous IL-10 locus. Auxiliary sequences (e.g., stop codons) can be added after the inserted sequence, or other methods (e.g., flipping or knocking out the endogenous IL-10RA gene sequence) can be applied so that the mouse endogenous IL-10RA protein cannot be expressed normally after the insertion site. Alternatively, in situ replacement can also be used, that is, the nucleotide sequence at the endogenous IL-10RA gene locus is directly substituted (e.g., replaced) with the human IL-10RA gene sequence. This example used in situ replacement to humanize the IL-10RA gene.

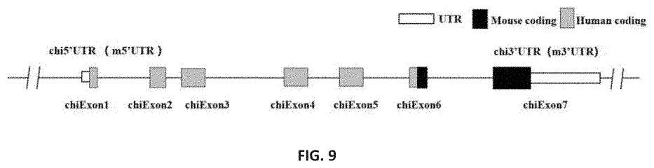

Specifically, a nucleic acid sequence of mouse IL-10RA gene can be replaced with a nucleic acid sequence of human IL-10RA gene at the endogenous IL-10 locus by gene editing technologies. For example, under control of a mouse IL-10RA regulatory element, a sequence of about 8702 bp spanning from exon 1 (including a part of exon 1) to exon 6 (including a part of exon 6) of mouse IL-10RA gene was replaced with a corresponding human DNA sequence to obtain a humanized IL-10RA locus, thereby humanizing mouse IL-10RA gene (shown in FIG. 9). The humanization allowed the mouse to transcribe a chimeric IL-10RA mRNA and express human IL10RA protein.

As shown in the schematic diagram of the targeting strategy in FIG. 10, a targeting vector was constructed, which contained the homologous arm sequences upstream and downstream of the endogenous mouse IL-10RA, and a “IL-10RA-A fragment” containing the human IL-10RA gene sequence. The upstream homologous arm (5′ homologous arm, SEQ ID NO: 29) contains a sequence 5705 bp upstream of the 5′UTR (including the 5′ UTR), and is identical to the nucleic acids 45274777-45269073 of NCBI Reference Number NC_000075.6. The downstream homologous arm (3′ homologous arm, SEQ ID NO: 30) contains a part of intron 6, and exon 7 (including 3′ UTR), with sequence identical to nucleic acids 45260102-45253456 of NCBI Reference Number NC_000075.6. The IL-10RA-A fragment contains a human IL-10RA gene fragment starting within exon 1 and ending within exon 6 of the human IL-10RA gene. The human IL-10 gene fragment is identical to nucleic acids 117986468-117995692 of NCBI Reference Number NC_000011.10. The 5′ end of human IL-10 gene fragment is directly connected to the 5′ homologous arm.

The connection between the 3′ end of the human IL-10RA gene fragment and the mouse IL-10RA gene locus was designed as

(SEQ ID NO: 53)
5′-CCCTCGCCTACTGCCTGGCCCTCCAGCTGTATGTG
CGGCGCCGAAAGAAGTTGCCTACAGTCCTGGTGAGTGC
TCTGTCCTTCCTGGATTCCC-3′,

wherein the “G” of sequence “AAAG” is the last nucleotide of the human sequence, and the first “A” of sequence “AAGT” is the first nucleotide of the mouse sequence.

The mRNA sequence of the engineered mouse IL-10RA after humanization and its encoded protein sequence are shown in SEQ ID NO: 31 and SEQ ID NO: 32, respectively. The targeting vector also included an antibiotic resistance gene for positive clone screening (neomycin phosphotransferase gene, or Neo), and two Frt recombination sites flanking the antibiotic resistance gene, that formed a Neo cassette. The Neo cassette is located between exon 6 and exon 7 of the mouse IL-10RA gene. The connection between the 5′ end of the Neo cassette and the mouse IL-10RA gene locus was designed as

(SEQ ID NO: 33)
5′-GTAATCATAGTTTCATCATCTACACAATGGCCTCG
AGGTCGACGGTATCGATAAGCTTGATATCGAATTCCGA
AGTTCCTATTCTCTAGA-3′,

wherein the “C” of sequence “TGGC” is the last nucleotide of the mouse sequence, and the first C of sequence “CTCG” is the first nucleotide of the Neo cassette. The connection between the 3′ end of the Neo cassette with the mouse IL-10RA gene locus was designed as

(SEQ ID NO: 34)
5′-AAGTATAGGAACTTCATCAGTCAGGTACATAATGG
TGGATCCCTGTGAACCGGCCACTGTGTATCATGAATTG
GTCCTGG-3′,

wherein the last “C” of sequence “ATCC” is the last nucleotide of the Neo cassette, and the “C” of sequence “CTGTG” is the first nucleotide of the mouse sequence. In addition, a coding gene with a negative selectable marker (a gene encoding diphtheria toxin A subunit (DTA)) was also inserted downstream of the 3′ homologous arm of the targeting vector.

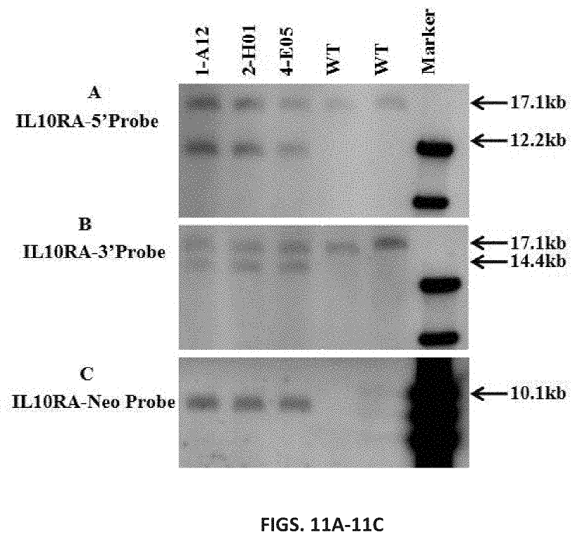

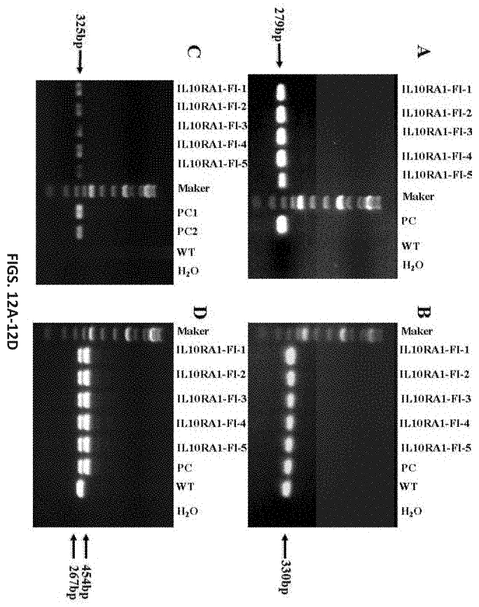

The targeting vector was constructed by restriction enzyme digestion and ligation. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot (digested with BamHI, SspI, and SpeI, respectively, and then hybridized with 3 probes) to screen out correct positive clone cells. The 3 probes were IL10RA-5′ Probe, c, and IL10RA-Neo Probe, as shown in the table below. As shown in FIGS. 11A-11C, the test results showed that the target band size of the three clones was correct, and no random insertions were detected. The results indicate that 1-A12, 2-H01, and 4-E05 were positive clones containing the correctly recombinant genome.

TABLE 5
Wild-type and target fragment size of specific probes

| | | WT size | Targeted size |
|---|---|---|---|
| Restriction | | (wild- | (successful |
| enzyme | Probe | type) | recombination) |
| BamHl | IL10RA-3′ Probe | 17.1 kb | 14.4 kb |
| Sspl | IL10RA-5′ Probe | 17.1 kb | 12.2 kb |
| Spel | ILI0RA-Neo Probe | — | 10.1 kb |

The following primers were used in PCR:

IL-10RA-F1:
(SEQ ID NO: 35)
5′-GGTCATTCTCCCGTAGGCCATGTTC-3′,
IL-10RA-R1:
(SEQ ID NO: 36)
5′-CCTTACCATGAGCGTCTGAGCCAAG-3′;
IL-10RA-F2:
(SEQ ID NO: 37)
5′-GCTCGACTAGAGCTTGCGGA-3′,
IL-10RA-R2:
(SEQ ID NO: 38)
5′-GGTCTTTCCCCAACAGCCTTTCAGA-3′.

The following probes were used in Southern Blot assays:

IL10RA-5′Probe:
F:
(SEQ ID NO: 39)
5′-AACCCACAACAGGGAACTGCAGAAG-3′,
R:
(SEQ ID NO: 40)
5′-GTTGCTGGGTTTCTAGACCAGAGCC-3′;
IL10RA-3′ Probe:
F:
(SEQ ID NO: 41)
5′-TGGCCTGCTAGAAATGCTTGTGAGA-3′,
R:
(SEQ ID NO: 42)
5′-GATCCCCAGCTTCCAAGATACTGGC-3′;
IL10RA-Neo Probe:
F:
(SEQ ID NO: 43)
5′-GGATCGGCCATTGAACAAGAT-3′,
R:
(SEQ ID NO: 44)
5′-CAGAAGAACTCGTCAAGAAGGC-3′.

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp mice to remove the positive selectable marker gene (FIG. 10), and then the humanized IL-10RA homozygous mice expressing humanized IL-10RA protein were obtained by breeding with each other. The genotype of the progeny mice can be identified by PCR with primers shown in the table below. The identification results of exemplary F1 generation mice (Neo cassette-removed) are shown in FIGS. 12A-12D, and mice labelled IL10RA1-F1-1, IL10RA1-F1-2, IL10RA1-F1-3, IL10RA1-F1-4, and IL10RA1-F1-5 were identified as positive heterozygous clones. Homozygous mice were made from these heterozygous mice. These mice were grossly normal.

The results indicated that this method can be used to construct genetically engineered IL10RA mice and the genetic modification can be stably passed to the next generation without random insertions.

TABLE 6

| | | | Fragment |
|---|---|---|---|
| Primer | SEQ ID | Sequence | size |
| name | NO | (5′-3′) | (bp) |
| IL10RA- | SEQ ID | CCCAGAACTAGCT | Mut: 279 |
| WT-F | NO: 45 | AGACTTCGCTGC | |
| IL10RA- | SEQ ID | CCTTACCATGAGC | |
| Mut-R | NO: 47 | GTCTGAGCCAAG | |
| IL10RA- | SEQ ID | CCCAGAACTAGCT | WT: 330 |
| WT-F | NO: 45 | CAGACTTCGCTG | |
| IL10RA- | SEQ ID | CAGATTCTCCCGC | |
| WT-R1 | NO: 46 | GAATGTGAACT | |
| Flp-F2 | SEQ ID | GACAAGCGTTAGT | Mut: 325 bp |
| | NO: 50 | AGGCACATATAC | |
| Flp-R2 | SEQ ID | GCTCCAATTTCCC | (Neo cassette- |
| | NO: 51 | ACAACATTAGT | removed) |
| IL10RA- | SEQ ID | GTCCTGGTGAGTG | WT: 267 |
| Frt-F | NO: 48 | CTCTGTCCTTC | (Neo cassette- |
| | | | removed) |
| | | | Mut: 454 |
| IL10RA- | SEQ ID | GTGTGATCCTGTG | |
| Frt-R | NO: 49 | AGTGGAAGCAACC | |

Note:
WT: wild-type; Mut: target sequence.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric interleukin-10 receptor alpha subunit (IL10RA).

2. The animal of claim 1, wherein the sequence encoding the human or chimeric IL10RA is operably linked to an endogenous regulatory element at the endogenous IL10RA gene locus in the at least one chromosome.

3. The animal of claim 1, wherein the sequence encoding a human or chimeric IL10RA comprises a sequence encoding an amino acid sequence that is at least 70%, 75%, 80%, 85%, 90%, 95%, 99%, or 100% identical to human IL10RA (NP_001549.2; SEQ ID NO: 28).

4. The animal of claim 1, wherein the sequence encoding a human or chimeric IL10RA comprises a sequence encoding an amino acid sequence that is at least 70%, 75%, 80%, 85%, 90%, 95%, 99%, or 100% identical to SEQ ID NO: 32.

5. The animal of claim 1, wherein the sequence encoding a human or chimeric IL10RA comprises a sequence that is at least 70%, 75%, 80%, 85%, 90%, 95%, 99%, or 100% identical to amino acids 1-264 of SEQ ID NO: 28.

6. The animal of any one of claims 1-6, wherein the animal is a mammal, e.g., a monkey, a rodent or a mouse.

7. The animal of any one of claims 1-6, wherein the human or chimeric IL10RA forms a functional IL10R complex with an endogenous IL10Rb.

8. The animal of any one of claims 1-7, wherein the animal does not express endogenous IL10RA.

9. The animal of any one of claims 1-8, wherein the animal has one or more cells expressing human or chimeric IL10RA.

10. The animal of any one of claims 1-8, wherein the animal has one or more cells expressing human or chimeric IL10RA, and endogenous IL10 can bind to the IL10R complex comprising the expressed human or chimeric IL10RA.

11. The animal of any one of claims 1-8, wherein the animal has one or more cells expressing human or chimeric IL10RA, and human IL10 can bind to the IL10R complex comprising the expressed human or chimeric IL10RA.

12. A genetically-modified, non-human animal, wherein the genome of the animal comprises a replacement of a sequence encoding a region of endogenous IL10RA with a sequence encoding a corresponding region of human IL10RA at an endogenous IL10RA gene locus.

13. The animal of claim 12, wherein the sequence encoding the corresponding region of human IL10RA is operably linked to an endogenous regulatory element at the endogenous IL10RA locus, and one or more cells of the animal express a human or chimeric IL10RA.

14. The animal of claim 12, wherein the animal does not express endogenous IL10RA.

15. The animal of claim 12, wherein the replaced sequence encodes the extracellular region of IL10RA.

16. The animal of claim 12, wherein the replaced sequence encodes the extracellular region, the transmembrane region, and a portion of the transmembrane region of IL10RA.

17. The animal of claim 12, wherein the animal has one or more cells expressing a chimeric IL10RA having an extracellular region, wherein the extracellular region comprises a sequence that is at least 50%, 60%, 70%, 80%, 90%, 95%, or 99% identical to the extracellular region of human IL10RA.

18. The animal of claim 17, wherein the extracellular region of the chimeric IL10RA has a sequence that has at least 10, 20, 30, 40, 50, 60, 70, 80, 90, 100, or 150 contiguous amino acids that are identical to a contiguous sequence present in the extracellular region of human IL10RA.

19. The animal of claim 12, wherein the animal is a mouse, and the replaced endogenous IL10RA region is a portion of exon 1, exon 2, exon 3, exon 4, exon 5, and/or a portion of exon 6 of the endogenous mouse IL10RA gene.

20. The animal of claim 13, wherein the animal is heterozygous with respect to the insertion at the endogenous IL10RA gene locus.

21. The animal of claim 13, wherein the animal is homozygous with respect to the insertion at the endogenous IL10RA gene locus.

22. A method for making a genetically-modified, non-human animal, comprising:
replacing in at least one cell of the animal, at an endogenous IL10RA gene locus, a sequence encoding a region of an endogenous IL10RA with a sequence encoding a corresponding region of human IL10RA.

23. The method of claim 22, wherein the sequence encoding the corresponding region of human IL10RA comprises a portion of exon 1, exon 2, exon 3, exon 4, exon 5, and/or a portion of exon 6 of a human IL10RA gene.

24. The method of claim 22, wherein the sequence encoding the corresponding region of human IL10RA comprises at least 50, 100, 200, or 300 nucleotides of exon 1, exon 2, exon 3, exon 4, exon 5, and/or exon 6 of a human IL10RA gene.

25. The method of claim 22, wherein the sequence encoding the corresponding region of human IL10RA encodes a sequence that is at least 90% identical to amino acids 1-264 of SEQ ID NO: 28.

26. The method of claim 22, wherein the locus is located at the extracellular region of IL10RA.

27. The method of claim 22, wherein the locus comprises the sequence encodes the extracellular region and the transmembrane region of IL10RA.

28. The method of claim 22, wherein the animal is a mouse, and the locus is a portion of exon 1, exon 2, exon 3, exons 4, exon 5, and/or a portion of exon 6 of the mouse IL10RA gene.

29. A non-human animal comprising at least one cell comprising a nucleotide sequence encoding a chimeric IL10RA polypeptide, wherein the chimeric IL10RA polypeptide comprises at least 50 contiguous amino acid residues that are identical to the corresponding contiguous amino acid sequence of a human IL10RA, wherein the animal expresses the chimeric IL10RA.

30. The animal of claim 29, wherein the chimeric IL10RA polypeptide has at least 50 contiguous amino acid residues that are identical to the corresponding contiguous amino acid sequence of a human IL10RA extracellular region.

31. The animal of claim 29, wherein the chimeric IL10RA polypeptide comprises a sequence that is at least 90%, 95%, or 99% identical of SEQ ID NO: 32.

32. The animal of claim 29, wherein the nucleotide sequence is operably linked to an endogenous IL10RA regulatory element of the animal.

33. The animal of claim 29, wherein the nucleotide sequence is integrated to an endogenous IL10RA gene locus of the animal.

34. The animal of claim 29, wherein the chimeric IL10RA has at least one mouse IL10RA activity and/or at least one human IL10RA activity.

35. A method of making a genetically-modified mouse cell that expresses a chimeric IL10RA, the method comprising:
replacing, at an endogenous mouse IL10RA gene locus, a nucleotide sequence encoding a region of mouse IL10RA with a nucleotide sequence encoding a corresponding region of human IL10RA, thereby generating a genetically-modified mouse cell that includes a nucleotide sequence that encodes the chimeric IL10RA, wherein the mouse cell expresses the chimeric IL10RA.

36. The method of claim 35, wherein the chimeric IL10RA comprises:
an extracellular region of human IL10RA;
a transmembrane region of human IL10RA; and/or
a cytoplasmic region that is at least 90% identical to mouse IL10RA cytoplasmic region.

37. The method of claim 35, wherein the nucleotide sequence encoding the chimeric IL10RA is operably linked to an endogenous IL10RA regulatory region, e.g., promoter.

38. The animal of any one of claims 1-21 and 29-34, wherein the animal further comprises a sequence encoding an additional human or chimeric protein (e.g., IL10, IL3, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal regulatory protein α (SIRPa)).

39. The animal of claim 38, wherein the additional human or chimeric protein is IL10.

40. The method of any one of claims 22-28 and 35-37, wherein the animal or mouse further comprises a sequence encoding an additional human or chimeric protein (e.g., IL10, IL3, PD-1, CTLA-4, LAG-3, BTLA, PD-L1, CD27, CD28, TIGIT, TIM-3, GITR, CD137, OX40, CD47 or SIRPa).

41. The method of claim 40, wherein the additional human or chimeric protein is IL10.

42. A genetically-modified, non-human animal whose genome comprises at least one chromosome comprising a sequence encoding a human or chimeric IL10.

43. The animal of claim 42, wherein the sequence encoding the human or chimeric IL10 is operably linked to an endogenous regulatory element at the endogenous IL10 gene locus in the at least one chromosome.

44. The animal of claim 42, wherein the sequence encoding a human or chimeric IL10 comprises a sequence encoding an amino acid sequence that is at least 70%, 75%, 80%, 85%, 90%, 95%, 99%, or 100% identical to human IL10 (NP_000563.1; SEQ ID NO: 4).

45. The animal of claim 42, wherein the animal comprises a sequence that is at least 70%, 75%, 80%, 85%, 90%, 95%, 99%, or 100% identical to SEQ ID NO: 7.

46. The animal of claim 42, wherein the animal comprises a sequence that is at least 70%, 75%, 80%, 85%, 90%, 95%, 99%, or 100% identical to SEQ ID NO: 52.

47. The animal of claim 42, wherein the animal comprises a sequence that is at least 70%, 75%, 80%, 85%, 90%, 95%, 99%, or 100% identical to SEQ ID NO: 8 or SEQ ID NO: 9.

48. The animal of any one of claims 42-47, wherein the animal is a mammal, e.g., a monkey, a rodent or a mouse.

49. The animal of any one of claims 42-48, wherein the animal is a mouse.

50. The animal of any one of claims 42-49, wherein the animal does not express endogenous IL10, or expresses a decreased level of endogenous IL10 as compared to IL10 expression level in a wild-type animal.

51. The animal of any one of claims 42-50, wherein the animal has one or more cells expressing human IL10.

52. The animal of any one of claims 42-51, wherein the animal has one or more cells expressing human or chimeric IL10, and the expressed human or chimeric IL10 can bind to endogenous IL10R complex.

53. The animal of any one of claims 42-51, wherein the animal has one or more cells expressing human or chimeric IL10, and the expressed human or chimeric IL10 can bind to human IL10R complex.

54. A genetically-modified, non-human animal, wherein the genome of the animal comprises a replacement of a sequence encoding a region of endogenous IL10 with a sequence encoding a corresponding region of human IL10 at an endogenous IL10 gene locus.

55. The animal of claim 54, wherein the sequence encoding the corresponding region of human IL10 is operably linked to an endogenous regulatory element at the endogenous IL10 locus, and one or more cells of the animal expresses a human IL10.

56. The animal of claim 54, wherein the animal does not express endogenous IL10, or expresses a decreased level of endogenous IL10 as compared to IL10 expression level in a wild-type animal.

57. The animal of claim 54, wherein the replaced locus comprises a sequence that is at least 70%, 75%, 80%, 85%, 90%, 95%, 99%, or 100% identical to SEQ ID NO: 7, SEQ ID NO: 8, SEQ ID NO: 9, or SEQ ID NO: 52.

58. The animal of claim 54, wherein the animal is a mouse, and the replaced endogenous IL10 region is a portion of exon 1, exon 2, exon 3, exon 4 and/or a portion of exon 5 of the endogenous mouse IL10 gene.

59. The animal of claim 54, wherein the animal is heterozygous with respect to the replacement at the endogenous IL10 gene locus.

60. The animal of claim 54, wherein the animal is homozygous with respect to the replacement at the endogenous IL10 gene locus.

61. A method for making a genetically-modified, non-human animal, comprising:
replacing in at least one cell of the animal, at an endogenous IL10 gene locus, a sequence encoding a region of an endogenous IL10 with a sequence encoding a corresponding region of human IL10.

62. The method of claim 61, wherein the sequence encoding the corresponding region of human IL10 comprises a portion of exon 1, exon 2, exon 3, exon 4, and/or a portion of exon 5 of a human IL10 gene.

63. The method of claim 61, wherein the sequence encoding the corresponding region of IL10 comprises at least 30, 50, 75, 100, or 150 nucleotides of exon 1, exon 2, exon 3, exon 4, and/or exon 5 of a human IL10 gene.

64. The method of claim 61, wherein the sequence encoding the corresponding region of human IL10 encodes a sequence that is at least 90% identical to SEQ ID NO: 4.

65. The method of claim 61, wherein replaced locus comprises a sequence of SEQ ID NO: 7, SEQ ID NO: 8, SEQ ID NO: 9, or SEQ ID NO: 52.

66. The method of claim 61, wherein the animal is a mouse, and the locus is a portion of exon 1, exon 2, exon 3, exon 4, and a portion of exon 5 of the mouse IL10 gene.

67. A non-human animal comprising at least one cell comprising a nucleotide sequence encoding a human or chimeric IL10 polypeptide, wherein the human or chimeric IL10 polypeptide comprises at least 50 contiguous amino acid residues that are identical to the corresponding contiguous amino acid sequence of a human IL10, wherein the animal expresses the human or chimeric IL10.

68. The animal of claim 67, wherein the human or chimeric IL10 polypeptide has at least 100 contiguous amino acid residues that are identical to the corresponding contiguous amino acid sequence of a human IL10.

69. The animal of claim 67, wherein the nucleotide sequence is operably linked to an endogenous IL10 regulatory element of the animal.

70. The animal of claim 67, wherein the nucleotide sequence is integrated to an endogenous IL10 gene locus of the animal.

71. A method of making a genetically-modified mouse cell that expresses a human or chimeric IL10, the method comprising:
replacing, at an endogenous mouse IL10 gene locus, a nucleotide sequence encoding a region of mouse IL10 with a nucleotide sequence encoding a corresponding region of human IL10, thereby generating a genetically-modified mouse cell that includes a nucleotide sequence that encodes the human or chimeric IL10, wherein the mouse cell expresses the human or chimeric IL10.

72. The method of claim 71, wherein the nucleotide sequence encoding the human or chimeric IL10 is operably linked to an endogenous IL10 regulatory region, e.g., promoter.

73. The animal of any one of claims 42-60 and 67-70, wherein the animal further comprises a sequence encoding an additional human or chimeric protein (e.g., IL10R, IL10RA, IL10Rb, IL3, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or SIRPa).

74. The animal of claim 73, wherein the additional human or chimeric protein is IL10RA.

75. The method of any one of claims 61-66, 71, and 72, wherein the animal or mouse further comprises a sequence encoding an additional human or chimeric protein (e.g., IL10R, IL10RA, IL10Rb, IL3, PD-1, CTLA-4, LAG-3, BTLA, PD-L1, CD27, CD28, TIGIT, TIM-3, GITR, CD137, OX40, CD47, or SIRPa).

76. The method of claim 75, wherein the additional human or chimeric protein is IL10RA.

77. A method of determining effectiveness of an IL10/IL10R pathway modulator for treating an autoimmune disorder, comprising:
administering the IL10/IL10R pathway modulator to the animal of any one of claims 1-21, 29-34, 38, 39, 42-60, 67-70, 73 and 74; and
determining the effects of the IL10/IL10R pathway modulator.

78. The method of claim 77, wherein the IL10/IL10R pathway modulator is an anti-human IL10 antibody.

79. The method of claim 77, wherein the IL10/IL10R pathway modulator is an anti-human IL10R antibody.

80. The method of claim 77, wherein the autoimmune disorder is asthma, systemic lupus erythematosus (SLE), rheumatoid arthritis (RA), Sjogren's syndrome (SS), multiple sclerosis (MS), Crohn's disease (CD), inflammatory bowel disease (IBD), or psoriasis.

81. A method of determining effectiveness of an IL10/IL10R pathway modulator for reducing inflammation, comprising:
administering the IL10/IL10R pathway modulator to the animal of any one of claims 1-21, 29-34, 38, 39, 42-60, 67-70, 73 and 74; and
determining the effects of the IL10/IL10R pathway modulator.

82. A method of determining effectiveness of an IL10/IL10R pathway modulator for treating cancer, comprising:
administering the IL10/IL10R pathway modulator to the animal of any one of claims 1-21, 29-34, 38, 39, 42-60, 67-70, 73 and 74; and
determining the effects of the IL10/IL10R pathway modulator.

83. The method of claim 82, wherein the cancer is a solid tumor, colorectal cancer, melanoma, or pancreatic cancer.

84. A method of determining effectiveness of an IL10/IL10R pathway modulator for treating an infectious disease (e.g., tuberculosis), comprising:
administering the IL10/IL10R pathway modulator to the animal of any one of claims 1-21, 29-34, 38, 39, 42-60, 67-70, 73 and 74; and
determining the effects of the IL10/IL10R pathway modulator.

85. A method of determining toxicity of an anti-IL10R antibody or an anti-IL10 antibody, the method comprising
administering the anti-IL10R antibody or the anti-IL10 antibody to the animal of any one of claims 1-21, 29-34, 38, 39, 42-60, 67-70, 73 and 74; and
determining weight change of the animal.

86. The method of claim 85, wherein the method further comprises performing a blood test (e.g., determining red blood cell count).

87. A protein comprising an amino acid sequence, wherein the amino acid sequence is one of the following:
(a) an amino acid sequence set forth in SEQ ID NO: 32;
(b) an amino acid sequence that is at least 90%, 91%, 92%, 93%, 94%, 95%, 96%, 97%, 98%, or 99% identical to SEQ ID NO: 32;
(c) an amino acid sequence that is different from the amino acid sequence set forth in SEQ ID NO: 32 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 32.

88. A nucleic acid comprising a nucleotide sequence, wherein the nucleotide sequence is one of the following:
(a) a sequence that encodes the protein of claim 87;
(b) SEQ ID NO: 7, 8, 9, 31, 33, 34, 52, or 53; or
(c) a sequence that is at least 90%, 91%, 92%, 93%, 94%, 95%, 96%, 97%, 98%, or 99% identical to SEQ ID NO: 7, 8, 9, 31, 33, 34, 52, or 53.

89. A cell comprising the protein of claim 87 and/or the nucleic acid of claim 88.

90. An animal comprising the protein of claim 87 and/or the nucleic acid of claim 88.
